(19) United States
(12) Patent Application Publication
(10) Pub. No.: US 2017/0196854 A1
(43) Pub. Date: Jul. 13, 2017

(54) USE OF ANTHELMINTIC AGENTS AGAINST DIROFILARIA IMMITIS

(71) Applicants: INTERVET INTERNATIONAL B.V., Boxmeer (NL); Intervet Inc., Madison, NJ (US)

(72) Inventors: Christophe Pierre Alain Chassaing, Ingelheim am Rhein (DE); Jurgen Lutz, Wiesbaden (DE); Anja Regina Heckeroth, Stadecken-Elsheim (DE)

(73) Assignee: Intervet Inc., Madison, NJ (US)

(21) Appl. No.: 15/324,896

(22) PCT Filed: Jul. 10, 2015
(86) PCT No.: PCT/EP2015/065870

§ 371 Date: Jan. 9, 2017

(30) Foreign Application Priority Data
Jul. 11, 2014 (EP) 14176737.6

(51) Int. Cl. A61K 31/496; A61K 9/16

(52) U.S. Cl. CPC A61K 31/496; A61K 9/1629

(57) ABSTRACT

This invention is directed to compounds and salts that are generally useful as agents to treat an infection with Dirofilaria immitis. This invention also is directed to treatments comprising the administration of the compounds and salts to animals in need of the treatments.

### FIELD OF THE INVENTION

This invention relates to compounds and salts thereof that are generally useful as agents against Dirofilaria immitis. This invention also relates to treatments comprising the administration of the compounds and salts thereof to animals in need of the treatments.

### BACKGROUND OF THE INVENTION

Heartworm infection is caused by a filarial organism, Dirofilaria immitis. At least 70 species of mosquitoes can serve as intermediate hosts; Aedes, Anopheles and Culex are the most common genera acting as vectors. Patent infections are possible in numerous wild and companion animal species. Wild animal reservoirs include wolves, coyotes, foxes, California gray seals, sea lions, and raccoons. In companion animals, heartworm infection is diagnosed primarily in dogs and less commonly in cats and ferrets. Heartworm disease has been reported in most countries with temperate, semitropical, or tropical climates, including the USA, Canada, Australia, Latin America, and southern Europe. In companion animals, infection risk is greatest in dogs and cats housed outdoors, but any dog or cat, indoor or outdoor, is capable of being infected.

Mosquito vector species acquire microfilaria (a neonatal larval stage) while feeding on an infected host. Once ingested by the mosquito, development of microfilariae into the first larval stage (L1) occurs. They then actively molt into the second larval stage (L2) and again to the infective third stage (L3) within the mosquito in 1 to 4 weeks, depending on environmental temperatures. When mature, the infective larvae migrate to the labium of the mosquito. As the mosquito feeds, the infective larvae erupt through the tip of the labium with a small amount of hemolymph onto the host's skin. The larvae migrate into the bite wound, beginning the mammalian portion of their life cycle. In canids and other susceptible hosts, infective larvae (L3) molt into a fourth stage (L4) in 3 to 12 days. After remaining in the subcutaneous tissue, abdomen, and thorax for about 2 months, the L4 larvae undergo their final molt at day 50 to 70 into young adults, arriving in the heart and pulmonary arteries about 70 to 120 days following initial infection.

The only available heartworm adulticide is melarsomine dihydrochloride, which is effective against mature (adult) and immature heartworms of both genders. Heartworm infection is preventable with macrolide prophylaxis. Year-round prevention is advised because of the potential for severe consequences, regardless of the housing status of the animals. Formulations of the macrolide preventives ivermectin, milbemycin oxime, moxidectin, and selamectin are safe and effective as prescribed for all breeds of dogs. Ivermectin/pyrantel pamoate (hookworms and roundworms) and milbemycin (hookworms, roundworms, and whipworms) also provide control of intestinal nematodes. At the approved dose, milbemycin kills microfilariae quickly, and in the face of high microfilarial concentrations a shock reaction may occur. Thus, milbemycin should not be administered without close monitoring as a preventive in dogs with high numbers of microfilariae. Ivermectin for cats is safe and effective at 24 μg/kg, PO, once monthly. Formulations of selamectin and a combination of imidacloprid/moxidectin are labeled for both dogs and cats.

Inter alia due to the possibility of resistance against existing drugs, there is a continuous need for finding new drugs that are active against Dirofilaria immitis (which includes any of the non-adult animal stages of the organism), which drugs can be used to treat an infection therewith (which treatment may by to prevent the infection or to therapeutically reduce the infection).

### SUMMARY OF THE INVENTION

Briefly, this invention is related to compounds (and salts thereof) that can generally be used to treat an infection with Dirofilaria immitis. The compounds correspond in structure to Formula I:

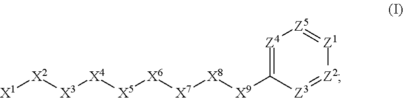

In Formula (I), X¹ is selected from the group consisting of C₃-C₆-alkyl, C₃-C₆-alkenyl, C₃-C₆-alkynyl, cyclopentyl, cyclohexyl, phenyl, 5-member heterocycloalkyl, 5-member heterocycloalkenyl, 5-member heteroaryl, 6-member heterocycloalkyl, 6-member heterocycloalkenyl, and 6-member heteroaryl. The C₃-C₆-alkyl, C₃-C₆-alkenyl, C₃-C₆-alkynyl, cyclopentyl, 5-member heterocycloalkyl, 5-member heterocycloalkenyl, and 5-member heteroaryl are optionally substituted by one or more substituents independently selected from the group consisting of halogen, cyano, alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl, wherein the alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl substituents are optionally substituted with one or more substituents independently selected from the group consisting of halogen, cyano, alkyl, alkoxy, haloalkyl, haloalkoxy, alkylsulfanyl, and haloalkylsulfanyl. The cyclohexyl, phenyl, 6-member heterocycloalkyl, 6-member heterocycloalkenyl, and 6-member heteroaryl are optionally substituted by one or more substituents independently selected from the group consisting of halogen, cyano, alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl, wherein the alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl substituents are optionally substituted with one or more substituents independently selected from the group consisting of halogen, cyano, alkyl, alkoxy, haloalkyl, haloalkoxy, alkylsulfanyl, and haloalkylsulfanyl.

X² is selected from the group consisting of a bond, —O—, —C(O)—, —C(S)—, —NH—, —S—, —S(O)—, —S(O)₂—, —CH₂—, —CH₂CH₂—, —C(O)—CH₂—, —CH₂—C(O)—, —O—CH₂—, —CH₂—O—, —NH—CH₂—, —CH₂—NH—, —S—CH₂—, —CH₂—S—, —S(O)—CH₂—, —CH₂—S(O)—, —S(O)₂—CH₂—, and —CH₂—S(O)₂—. The —NH— is optionally substituted with alkyl, and the —CH₂—, —CH₂CH₂—, —C(O)—CH₂—, —CH₂—C(O)—, —O—CH₂—, —CH₂—O—, —NH—CH₂—, —CH₂—NH—, —S—CH₂—, —CH₂—S—, —S(O)—CH₂—, —CH₂—S(O)—, —S(O)₂—CH₂—, and —CH₂—S(O)₂— are optionally substituted with one or more independently selected alkyl;

X³ is a linker, wherein the linker is a hydrocarbon wherein the linker comprises one or more nitrogen atoms, and one or more of the carbons in the hydrocarbon are optionally substituted with one or more substituents independently selected from the group consisting of halogen, alkyl, alkoxy, and oxo, the linker comprises at least one chain of from 3 to 6 atoms that link X² to X⁴, wherein from 1 to 2 of the chain atoms are nitrogen, and the linker comprises no chain of less than 3 atoms that links X² and X⁴.

X⁴ is selected from the group consisting of a bond, —CH₂—, —O—, —C(S)—, —C(O)—, —S(O)—, and —S(O)₂—, wherein the —CH₂— is optionally substituted with up to two substituents independently selected from the group consisting of alkyl, alkenyl, and carbocyclyl.

X⁵ is selected from the group consisting of a bond, —CH₂—, and carbocyclyl, wherein the —CH₂— is optionally substituted with up to two substituents independently selected from the group consisting of alkyl, alkenyl, and carbocyclyl.

X⁶ is selected from the group consisting of a bond, —CH₂—, and carbocyclyl, wherein the —CH₂— is optionally substituted with up to two substituents independently selected from the group consisting of alkyl, alkenyl, and carbocyclyl.

X⁷ is selected from the group consisting of —CH₂—, —O—, —C(O)—, —C(S)—, —S—, —S(O)—, —S(O)₂—, —NH—, —C(O)—NH—, —C(S)—NH—, —NH—C(O)—, —NH—C(S)—, wherein the —CH₂— is optionally substituted with up to two substituents independently selected from the group consisting of alkyl, alkenyl, and carbocyclyl, and any —NH— is optionally substituted at a substitutable position with a substituent selected from the group consisting of alkyl, alkenyl, alkynyl, alkoxyalkyl, carbocyclyl, and carbocyclylalkyl, wherein any such substituent is optionally substituted with one or more independently selected halogen.

X⁸ is selected from the group consisting of piperidinyl, piperazinyl, homopiperazinyl, and pyrrolidinyl, wherein the piperidinyl, piperazinyl, homopiperazinyl or pyrrolidinyl is optionally substituted with one or more independently selected alkyl;

X⁴—X⁵—X⁶—X⁷ comprises no chain of less than 3 atoms that links X³ to X⁸.

X⁹ is selected from the group consisting of a bond, —O—, —C(O)—, —S—, —S(O)—, —S(O)₂—, and —NH—, wherein the —NH— is optionally substituted at a substitutable position with a substituent selected from the group consisting of alkyl, alkenyl, alkynyl, alkoxyalkyl, carbocyclyl, and carbocyclylalkyl, wherein any such substituent is optionally substituted with one or more independently selected halogen.

Z¹ is selected from the group consisting of N and CH, wherein the CH is optionally substituted with a substituent selected from the group consisting of halogen, nitro, cyano, aminosulfonyl, alkyl, alkoxy, alkoxycarbonyl, alkylsulfanyl, alkylsulfinyl, alkylsulfonyl, aryl, arylsulfanyl, arylsulfinyl, arylsulfonyl, heteroaryl, heteroarylsulfanyl, heteroarylsulfinyl, and heteroarylsulfonyl, wherein the alkyl, alkoxy, alkoxycarbonyl, alkylsulfanyl, alkylsulfinyl, alkylsulfonyl, aryl, arylsulfanyl, arylsulfinyl, arylsulfonyl, heteroaryl, heteroarylsulfanyl, heteroarylsulfinyl, and heteroarylsulfonyl are optionally substituted with one or more substituents independently selected from the group consisting of halogen and alkyl, and the aminosulfonyl is optionally substituted with up to two independently selected alkyl.

Z² is selected from the group consisting of N and CH, wherein the CH is optionally substituted with a substituent selected from the group consisting of cyano, halogen, nitro, alkyl, alkoxy, haloalkyl, alkylsulfanyl, and haloalkylsulfanyl.

Z³, Z⁴, and Z⁵ are each independently selected from the group consisting of N and CH, wherein the CH is optionally substituted with a substituent selected from the group consisting of halogen, cyano, nitro, alkyl, alkoxy, alkylsulfanyl, haloalkyl, haloalkoxy, and haloalkylsulfanyl; and only one of Z¹, Z², Z³, Z⁴, and Z⁵ may be N.

This invention also is directed, in part, to methods for treating a disease in an animal, particularly an infection with Dirofilaria immitis. The methods comprise administering at least one compound or salt of this invention to the animal.

This invention also is directed, in part, to a kit. The kit comprises at least one compound or salt of this invention packed in a container (vial, bag, box, sachet, syringe, blister etc.). In addition, the kit comprises at least one other component, such as another ingredient (e.g., an excipient or active ingredient, i.e. an ingredient being suitable for any medical use, preferably an anthelminthic ingredient), instructions and/or an apparatus for combining the compound or salt with another ingredient, instructions and/or an apparatus for administering the compound or salt, and/or a diagnostic tool.

It is noted that the compounds for use in the present invention may also be used to treat a helminth infection caused by one or more helminths selected from the group consisting of Anaplocephala spp.; Dipylidium spp; Diphyllobothrium spp.; Echinococcus spp.; Moniezia spp.; Taenia spp.; Dicrocoelium spp.; Fasciola spp.; Paramphistomum spp.; Schistosoma spp.; Ancylostoma spp.; Anecator spp.; Ascaridia spp.; Ascaris spp.; Brugia spp.; Bunostomum spp.; Capillaria spp.; Chabertia spp.; Cooperia spp.; Cyathostomum spp.; Cylicocyclus spp.; Cylicodontophorus spp.; Cylicostephanus spp.; Craterostomum spp.; Dictyocaulus spp.; Dipetalonema spp.; Dirofilaria spp.; Dracunculus spp.; Enterobius spp.; Filaroides spp.; Habronema spp.; Haemonchus spp.; Heterakis spp.; Hyostrongylus spp.; Metastrongylus spp.; Meullerius spp.; Necator spp.; Nematodirus spp.; Nippostrongylus spp.; Oesophagostomum spp.; Onchocerca spp.; Ostertagia spp.; Oxyuris spp.; Parascaris spp.; Stephanurus spp.; Strongylus spp.; Syngamus spp.; Toxocara spp.; Strongyloides spp.; Teladorsagia spp.; Toxascaris spp.; Trichinella spp.; Trichuris spp.; Trichostrongylus spp.; Triodontophorous spp.; Uncinaria spp. and Wuchereria spp.

Further benefits of Applicants' invention will be apparent to one skilled in the art from reading this specification.

### DETAILED DESCRIPTION OF PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

### I. Compounds for Use in the Invention

The compounds for use in the present invention generally correspond in structure to Formula (I):

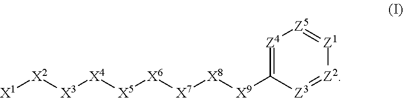

The substituents in Formula (I) are defined as follows:

### A. Preferred Embodiments of X¹

X¹ is selected from the group consisting of C₃-C₆-alkyl, C₃-C₆-alkenyl, C₃-C₆-alkynyl, cyclopentyl, cyclohexyl, phenyl, 5-member heterocycloalkyl, 5-member heterocycloalkenyl, 5-member heteroaryl, 6-member heterocycloalkyl, 6-member heterocycloalkenyl, and 6-member heteroaryl.

The C₃-C₆-alkyl, C₃-C₆-alkenyl, C₃-C₆-alkynyl, cyclopentyl, 5-member heterocycloalkyl, 5-member heterocycloalkenyl, and 5-member heteroaryl are optionally substituted by one or more substituents independently selected from the group consisting of halogen, cyano, alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl. The alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl substituents are optionally substituted with one or more substituents independently selected from the group consisting of halogen, cyano, alkyl, alkoxy, haloalkyl, haloalkoxy, alkylsulfanyl, and haloalkylsulfanyl.

The cyclohexyl, phenyl, 6-member heterocycloalkyl, 6-member heterocycloalkenyl, and 6-member heteroaryl are optionally substituted by one or more substituents independently selected from the group consisting of halogen, cyano, alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl. The alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl substituents are optionally substituted with one or more substituents independently selected from the group consisting of halogen, cyano, alkyl, alkoxy, haloalkyl, haloalkoxy, alkylsulfanyl, and haloalkylsulfanyl.

In some embodiments, the cyclohexyl, phenyl, 6-member heterocycloalkyl, 6-member heterocycloalkenyl, and 6-member heteroaryl are optionally substituted at the meta and para positions by one or more substituents independently selected from the group consisting of halogen, cyano, alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl. The alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl substituents are optionally substituted with one or more substituents independently selected from the group consisting of halogen, cyano, alkyl, alkoxy, haloalkyl, haloalkoxy, alkylsulfanyl, and haloalkylsulfanyl. The cyclohexyl, phenyl, 6-member heterocycloalkyl, 6-member heterocycloalkenyl, 6-member heteroaryl are optionally substituted at the ortho positions by one or more independently selected halogen.

In some embodiments, X¹ is C₃-C₆-alkyl.

In some embodiments, X¹ is C₃-C₄-alkyl.

In some embodiments, X¹ is C₃-alkyl. In some such embodiments, X¹ is isopropyl. In these embodiments, the compound is encompassed by the following formula:

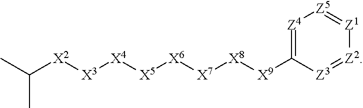

In some embodiments, X¹ is C₄-alkyl. In some such embodiments, X¹ is butyl. In such embodiments, the compound is encompassed by the following formula:

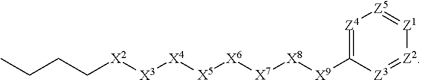

In some embodiments, X¹ is C₃-C₆-cycloalkyl. In some such embodiments, for example, X¹ is C₆-cycloalkyl (i.e., cyclohexyl). In such embodiments, the compound is encompassed by the following formula:

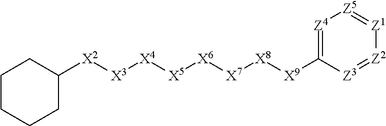

In some embodiments, X¹ is phenyl optionally substituted at the meta and para positions with one or more substituents selected from the group consisting of halogen, cyano, alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl. The alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl substituents are optionally substituted with one or more substituents independently selected from the group consisting of halogen, cyano, alkyl, alkoxy, haloalkyl, haloalkoxy, alkylsulfanyl, and haloalkylsulfanyl. The phenyl is also optionally substituted at the ortho positions by one or more independently selected halogen.

In some embodiments, X¹ is phenyl. In such embodiments, the compound is encompassed by the following formula:

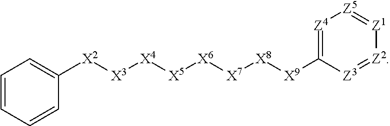

In some embodiments, X¹ is phenyl substituted with one substituent.

In some embodiments, X¹ is phenyl substituted with one substituent at an ortho position.

In some embodiments, X¹ is phenyl substituted with one halogen substituent at an ortho position. In some such embodiments, X¹ is phenyl substituted with chloro at an ortho position. Such embodiments are encompassed by the following formula:

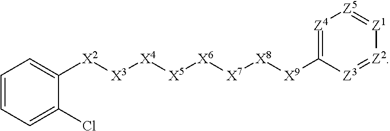

In some embodiments, X¹ is phenyl substituted with one substituent at a meta position.

In some embodiments, X¹ is phenyl substituted with haloalkyl at a meta position. In some such embodiments X¹ is phenyl substituted with trifluoromethyl at a meta position. Such embodiments are encompassed by the following formula:

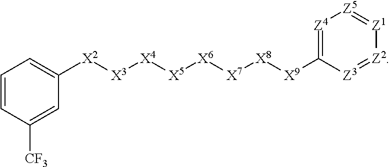

In other such embodiments, X¹ is phenyl substituted with chloro at a meta position. In such embodiments, the compound is encompassed by the following formula:

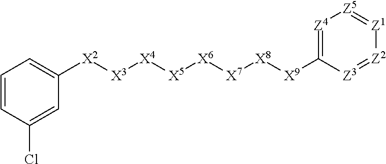

In other such embodiments, X¹ is phenyl substituted with halo-C₁-C₆-alkoxy at a meta position. In some such embodiments, for example, X¹ is phenyl substituted with fluoro-C₁-alkoxy (i.e., —OCF₃). Such embodiments are encompassed by the following formula:

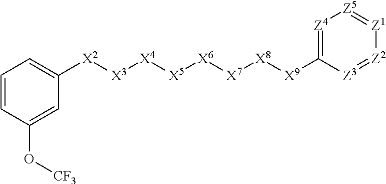

In some embodiments, X¹ is phenyl substituted with one substituent at the para position.

In some embodiments, X¹ is phenyl substituted with halo-C₁-C₆-alkyl at the para position. In some such embodiments, for example, X¹ is phenyl substituted with trifluoromethyl (i.e., —CF₃). at the para position. Such embodiments are encompassed by the following formula:

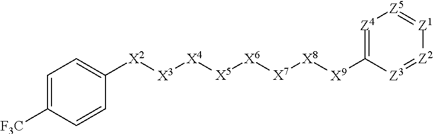

In some embodiments, X¹ is phenyl substituted with C₁-C₆-alkyl. In some such embodiments, for example, X¹ is phenyl substituted with tert-butyl at the para position. Such embodiments are encompassed by the following formula:

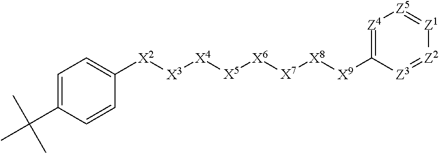

In other such embodiments, X¹ is phenyl substituted with C₃-alkyl (i.e. propyl) at the para position. In such embodiments, the compound is encompassed by the following formula:

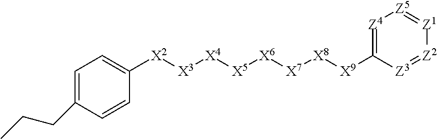

In yet other such embodiments, X¹ is phenyl substituted with C₁-alkyl (i.e. methyl) at the para position. In such embodiments, the compound is encompassed by the following formula:

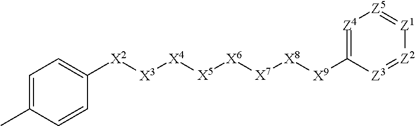

In some embodiments, X¹ is phenyl substituted with halo at the para position. In some such embodiments, for example, X¹ is phenyl substituted with chloro at the para position. Such embodiments are encompassed by the following formula:

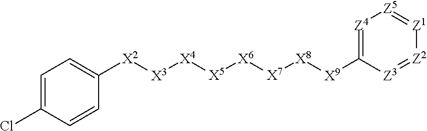

In other such embodiments, X¹ is phenyl substituted with fluoro at the para position. In such embodiments, the compound is encompassed by the following formula:

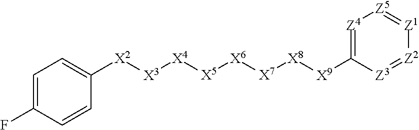

In some embodiments, X¹ is phenyl substituted with C₁-C₆-alkoxy. In some such embodiments, for example, X¹ is phenyl substituted with C₂-alkoxy (i.e. ethoxy) at the para position. Such embodiments are encompassed by the following formula:

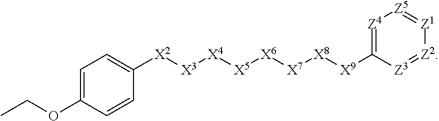

In some such embodiments, for example, X¹ is phenyl substituted with C₁-alkoxy (i.e. methoxy) at the para position. Such embodiments are encompassed by the following formula:

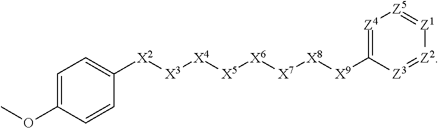

In some embodiments, X¹ is phenyl substituted with cyano at the para position. In those embodiments, the compound is encompassed by the following formula:

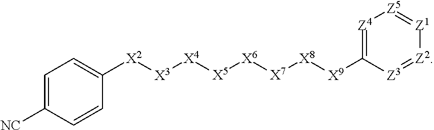

In some embodiments, X¹ is phenyl substituted with aryl at the para position. In some such embodiments, for example, X¹ is phenyl substituted with phenyl at the para position. Such embodiments are encompassed by the following formula:

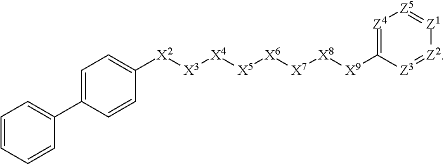

In some embodiments, X¹ is phenyl substituted with aryloxy at the para position. In some such embodiments, for example, X¹ is phenyl substituted with phenoxy at the para position. Such embodiments are encompassed by the following formula:

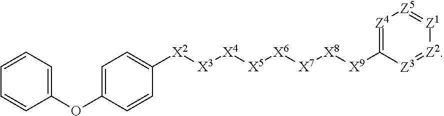

In some embodiments, X¹ is phenyl substituted with aryl-C₁-C₆-alkoxy at the para position. In some such embodiments, for example, X¹ is phenyl substituted with phenylmethoxy at the para position. Such embodiments are encompassed by the following formula:

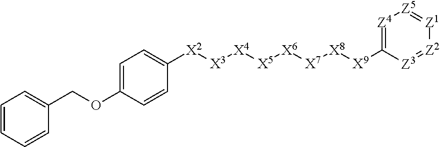

In some embodiments, X¹ is phenyl substituted C₁-C₆-alkoxy. In some such embodiments, for example, X¹ is phenyl para-substituted with C₄-alkoxy (i.e., isobutyloxy). Such embodiments are encompassed by the following formula:

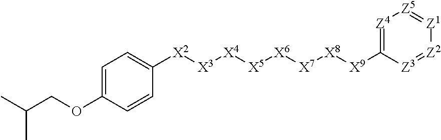

In some embodiments, X¹ is phenyl substituted with halo-C₁-C₆-alkyl-aryl-C₁-C₆-alkoxy. In some such embodiments, for example, X¹ is phenyl substituted with triflouro-C₁-alkylphenyl-C₁-alkoxy (i.e., trifluoromethylphenylmethoxy). Such embodiments are encompassed by the following formula:

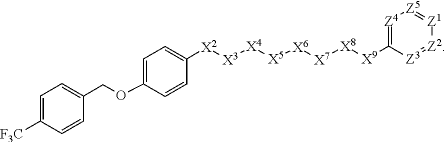

In some embodiments, X¹ is phenyl substituted with two substituents.

In some embodiments, X¹ is phenyl substituted at the ortho and para positions.

In some embodiments, X¹ is phenyl substituted at the ortho and para positions with two independently selected halo substituents. In some such embodiments, for example, X¹ is phenyl substituted with two chloro substituents. Such embodiments are encompassed by the following formula:

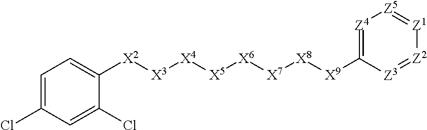

In other such embodiments, for example, X¹ is phenyl substituted with two fluoro substituents. Such embodiments are encompassed by the following formula:

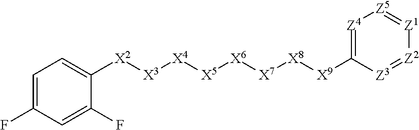

In yet other such embodiments, for example, X¹ is phenyl substituted with a fluoro at the ortho position and a chloro at the para position. Such embodiments are encompassed by the following formula:

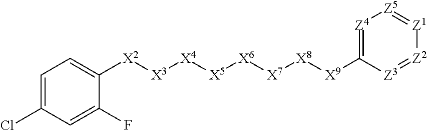

In some embodiments, X¹ is phenyl substituted at the meta and para positions.

In some embodiments, X¹ is phenyl substituted at meta and para positions. In some such embodiments, for example, X¹ is phenyl substituted with two chloro substituents. Such embodiments are encompassed by the following formula:

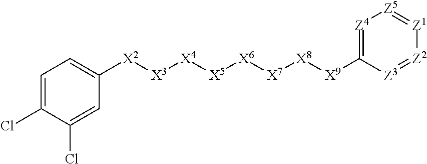

In other such embodiments, for example, X¹ is phenyl substituted with two independently selected C₁-C₆-alkoxy substituents. For example, X¹ is phenyl substituted with two C₁-alkoxy substituents (i.e., methoxy). Such embodiments are encompassed by the following formula:

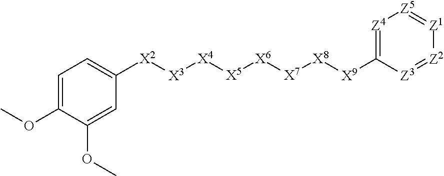

In other such embodiments, the compound corresponds in structure to the following formula:

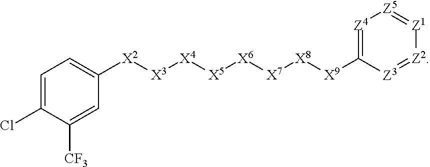

In yet other such embodiments, the compound corresponds in structure to the following formula:

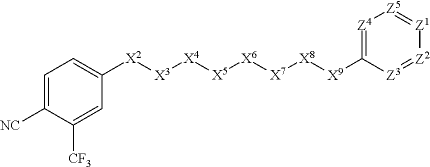

In some embodiments, X¹ is phenyl substituted at both meta positions.

In some embodiments, X¹ is phenyl substituted with two halo-C₁-C₆-alkyl substituents. For example, some such embodiments are encompassed by the following formula:

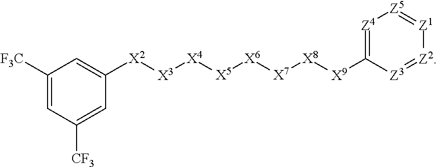

In some embodiments, X¹ is 5-membered heteroaryl optionally substituted by one or more substituents independently selected from the group consisting of halogen, cyano, alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl. The alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl substituents are optionally substituted with one or more substituents independently selected from the group consisting of halogen, cyano, alkyl, alkoxy, haloalkyl, haloalkoxy, alkylsulfanyl, and haloalkylsulfanyl.

In some embodiments, X¹ is optionally substituted thiadiazoyl, optionally substituted with a haloalkyl substituent. In some such embodiments, X¹ is thiadiazoyl substituted with trifluoromethyl. In such embodiments, the compound is encompassed by the following formula:

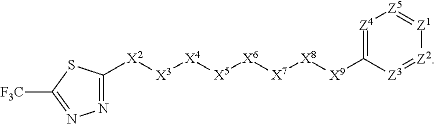

In some embodiments, X¹ is 6-membered heteroaryl optionally substituted by one or more substituents independently selected from the group consisting of halogen, cyano, alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl. The alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl substituents are optionally substituted with one or more substituents independently selected from the group consisting of halogen, cyano, alkyl, alkoxy, haloalkyl, haloalkoxy, alkylsulfanyl, and haloalkylsulfanyl. The cyclohexyl, phenyl, 6-member heterocycloalkyl, 6-member heterocycloalkenyl, 6-member heteroaryl are optionally substituted at the ortho positions by one or more independently selected halogen.

In some embodiments, X¹ is optionally substituted pyridinyl.

In some embodiments, X¹ is 2-pyridinyl. In such embodiments, the compound is encompassed by the following formula:

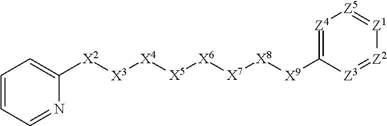

In some embodiments, X¹ is 2-pyridinyl substituted with haloalkyl. In such embodiments, the compound is encompassed by the following formula:

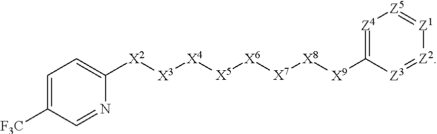

In some embodiments, X¹ is 2-pyridinyl substituted with chloro at the para position. In such embodiments, the compound is encompassed by the following formula:

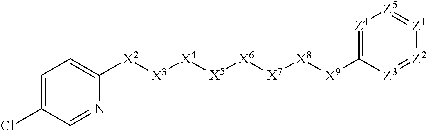

In some embodiments, X¹ is 3-pyridinyl. In such embodiments, the compound is encompassed by the following formula:

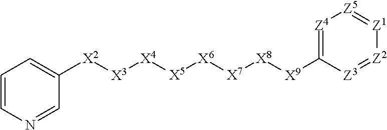

In some embodiments, X¹ is 3-pyridinyl substituted with halo-C₁-C₆-alkyl. In such embodiments, for example, the compound is encompassed by the following formula:

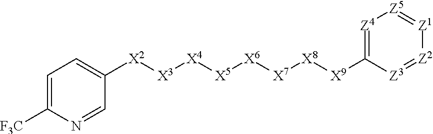

In some embodiments, X¹ is 3-pyridinyl substituted with C₁-C₆-alkoxy. In such embodiments, for example, the compound is encompassed by the following formula:

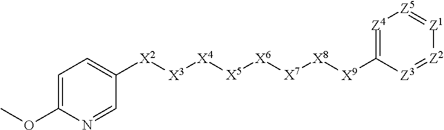

In yet other such embodiments, X¹ is 4-pyridinyl. In such embodiments, the compound is encompassed by the following formula:

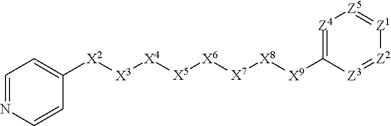

### B. Preferred Embodiments of X²

X² is selected from the group consisting of a bond, —O—, —C(O)—, —C(S)—, —NH—, —S—, —S(O)—, —S(O)₂—, —CH₂—, —CH₂CH₂—, —C(O)—CH₂—, —CH₂—C(O)—, —O—CH₂—, —CH₂—O—, —NH—CH₂—, —CH₂—NH—, —S—CH₂—, —CH₂—S—, —S(O)—CH₂—, —CH₂—S(O)—, —S(O)₂—CH₂—, and —CH₂—S(O)₂—. Here, the —NH— is optionally substituted with alkyl. The —CH₂—, —CH₂CH₂—, —C(O)—CH₂—, —CH₂—C(O)—, —O—CH₂—, —CH₂—O—, —NH—CH₂—, —CH₂—NH—, —S—CH₂—, —CH₂—S—, —S(O)—CH₂—, —CH₂—S(O)—, —S(O)₂—CH₂—, and —CH₂—S(O)₂— are optionally substituted with one or more independently selected alkyl.

In some embodiments, X² is selected from the group consisting of a bond, —O—, —C(O)—, —C(S)—, —NH—, —S—, —S(O)—, —S(O)₂—, —CH₂—, —CH₂CH₂—, —C(O)—CH₂—, —CH₂—C(O)—, —O—CH₂—, —CH₂—O—, —NH—CH₂—, —CH₂—NH—, —S—CH₂—, —CH₂—S—, —S(O)—CH₂—, —CH₂—S(O)—, —S(O)₂—CH₂—, and —CH₂—S(O)₂—. Here, the —NH— is optionally substituted with C₁-C₆-alkyl. The —CH₂—, —CH₂CH₂—, —C(O)—CH₂—, —CH₂—C(O)—, —O—CH₂—, —CH₂—O—, —NH—CH₂—, —CH₂—NH—, —S—CH₂—, —CH₂—S—, —S(O)—CH₂—, —CH₂—S(O)—, —S(O)₂—CH₂—, and —CH₂—S(O)₂— are optionally substituted with one or more independently selected C₁-C₆-alkyl.

In some embodiments, X² is a single bond. In such embodiments, the compound is encompassed by the following formula:

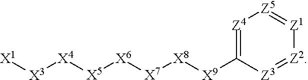

In some embodiments, X² is —O—. In such embodiments, the compound is encompassed by the following formula:

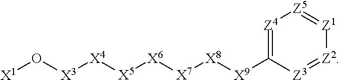

In some embodiments, X² is —C(O)—. In such embodiments, the compound is encompassed by the following formula:

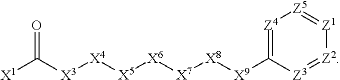

In some embodiments, X² is —C(S)—. In such embodiments, the compound is encompassed by the following formula:

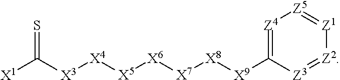

In some embodiments, X² is —NH—. In such embodiments, the compound is encompassed by the following formula:

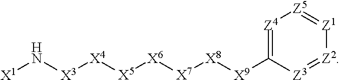

In some embodiments, X² is —S—. In such embodiments, the compound is encompassed by the following formula:

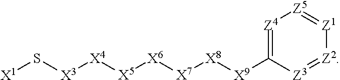

In some embodiments, X² is —S(O)—. In such embodiments, the compound is encompassed by the following formula:

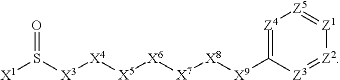

In some embodiments, X² is —S(O)₂—. In such embodiments, the compound is encompassed by the following formula:

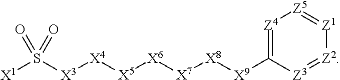

In some embodiments, X² is —CH₂—. In such embodiments, the compound is encompassed by the following formula:

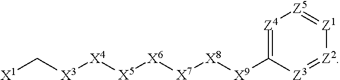

In some embodiments, X² is —CH₂CH₂—. In such embodiments, the compound is encompassed by the following formula:

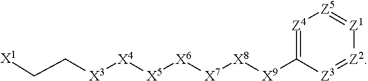

In some embodiments, X² is —C(O)—CH₂—. In such embodiments, the compound is encompassed by the following formula:

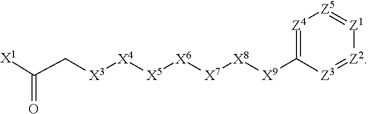

In some embodiments, X² is —CH₂—C(O)—. In such embodiments, the compound is encompassed by the following formula:

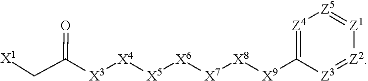

In some embodiments, X² is —O—CH₂—. In such embodiments, the compound is encompassed by the following formula:

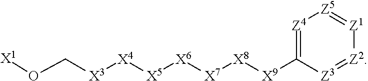

In some embodiments, X² is —CH₂—O—. In such embodiments, the compound is encompassed by the following formula:

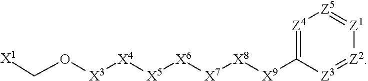

In some embodiments, X² is —NH—CH₂—. In such embodiments, the compound is encompassed by the following formula:

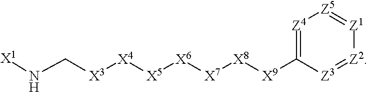

In some embodiments, X² is —CH₂NH—. In such embodiments, the compound is encompassed by the following formula:

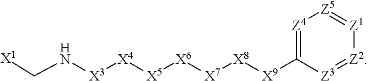

In some embodiments, X² is —S—CH₂—. In such embodiments, the compound is encompassed by the following formula:

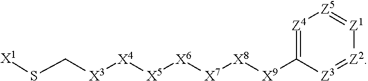

In some embodiments, X² is —CH₂—S—. In such embodiments, the compound is encompassed by the following formula:

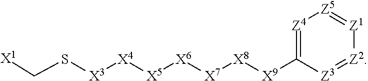

In some embodiments, X² is —S(O)—CH₂—. In such embodiments, the compound is encompassed by the following formula:

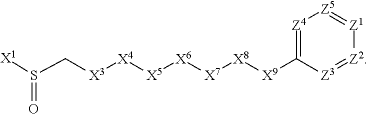

In some embodiments, X² is —CH₂—S(O)—. In such embodiments, the compound is encompassed by the following formula:

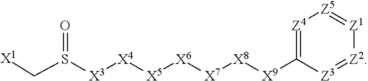

In some embodiments, X² is —S(O)₂—CH₂—. In such embodiments, the compound is encompassed by the following formula:

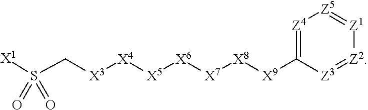

In some embodiments, X² is —CH₂—S(O)₂—. In such embodiments, the compound is encompassed by the following formula:

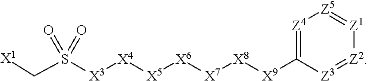

### C. Preferred Embodiments of X³

X³ is a linker. The linker is a hydrocarbon group, except: (a) the linker comprises one or more nitrogen atoms, and (b) one or more of the carbons in the hydrocarbon optionally are substituted with one or more substituents independently selected from the group consisting of oxo, halogen, hydroxy, alkyl, and alkoxy. The linker comprises at least one chain of from 3 to 6 atoms that bridges X² to X⁴. From 1 to 2 of the chain atoms are nitrogen. The linker has no chain of less than 3 atoms that bridges X² and X⁴.

In some embodiments, the linker is a hydrocarbon group, except: (a) the linker comprises one or more nitrogen atoms, and (b) one or more of the carbons in the hydrocarbon optionally are substituted with one or more substituents independently selected from the group consisting of oxo, halogen, alkyl, and alkoxy. The linker comprises at least one chain of from 3 to 5 atoms that bridges X² to X⁴. From 1 to 2 of the chain atoms are nitrogen. The linker has no chain of less than 3 atoms that bridges X² and X⁴.

In some embodiments, the linker is a hydrocarbon group, except: (a) the linker comprises one or more nitrogen atoms, and (b) one or more of the carbons in the hydrocarbon optionally are substituted with one or more substituents independently selected from the group consisting of oxo, halogen, hydroxy, C₁-C₆-alkyl, and C₁-C₆-alkoxy.

In some embodiments, the linker is a hydrocarbon group, except: (a) the linker comprises one or more nitrogen atoms, and (b) one or more of the carbons in the hydrocarbon optionally are substituted with oxo.

In some embodiments, the linker is a hydrocarbon group, except: (a) the linker comprises one or more nitrogen atoms, and (b) one carbon in the hydrocarbon is substituted with oxo.

In some embodiments, the linker is a hydrocarbon group, except for comprising one or more nitrogen atoms.

In some embodiments, the linker comprises no greater than one nitrogen atom.

In other embodiments, the linker comprises no greater and no less than two nitrogen atoms.

In some embodiments, the linker comprises at least one chain of from 3 to 6 atoms that bridges X² to X⁴.

In some embodiments, the linker comprises at least one 3-atom chain that bridges X² to X⁴.

In some embodiments, the linker comprises at least one 4-atom chain that bridges X² to X⁴. In some such embodiments, the linker has no chain of less than 4 atoms that bridges X² to X⁴.

In some embodiments, the linker comprises at least one 5-atom chain that bridges X² to X⁴. In some such embodiments, the linker has no chain of less than 5 atoms that bridges X² to X⁴.

In some embodiments, X³ is selected from the group of linkers consisting of those shown in Table I:

TABLE I
Example of X³ Linkers

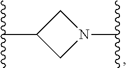 [SMILES: *C1CN(*)C1]

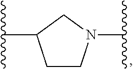 [SMILES: *C1CCN(*)C1]

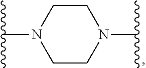 [SMILES: *N1CCN(*)CC1]

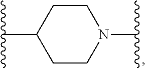 [SMILES: *C1CCN(*)CC1]

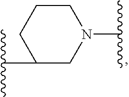 [SMILES: *C1CCCN(*)C1]

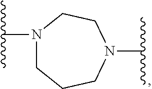 [SMILES: *N1CCCN(*)CC1]

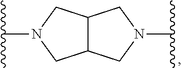 [SMILES: *N1CC2CN(*)CC2C1]

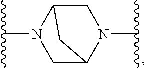 [SMILES: *N1CC2CC1CN2*]

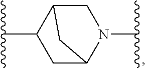 [SMILES: *C1CC2CC1CN2*]

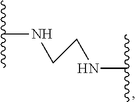 [SMILES: *NCCN*]

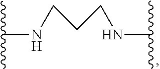 [SMILES: *NCCCN*]

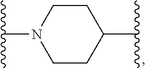 [SMILES: *C1CCN(*)CC1]

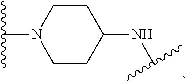 [SMILES: *NC1CCN(*)CC1]

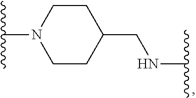 [SMILES: *NCC1CCN(*)CC1]

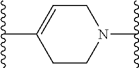 [SMILES: *C1=CCN(*)CC1]

Any such group is optionally substituted with one or more substituents independently selected from the group consisting of halogen, C₁-C₆-alkyl, C₁-C₆-alkoxy, oxo, and thiocarbonyl.

In some embodiments, X³ is selected from the group consisting of:

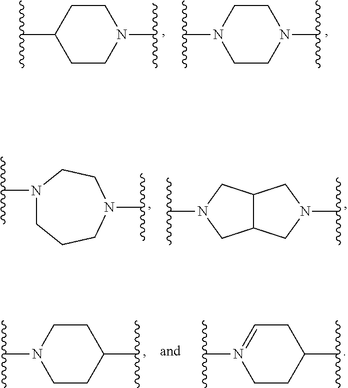

In some embodiments, the linker comprises at least one 3-atom chain that bridges X² to X⁴. To illustrate, the following are some of the structures from Table I that exemplify such linkers:

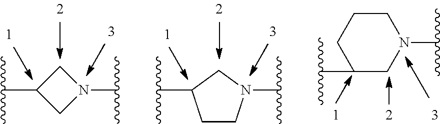

In some embodiments, the linker comprises at least one 4-atom chain that bridges X² to X⁴. To illustrate, the following are some of the structures from Table I that exemplify such linkers:

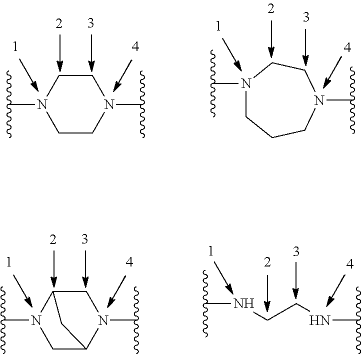 [SMILES: *N1CCN(*)CC1 ; *N1CCCN(*)CC1 ; *N1CC2CC1CN2* ; *NCCN*]

In some embodiments, the linker comprises at least one 5-atom chain that bridges X² to X⁴. To illustrate, the following are some of the structures from Table I that exemplify such linkers:

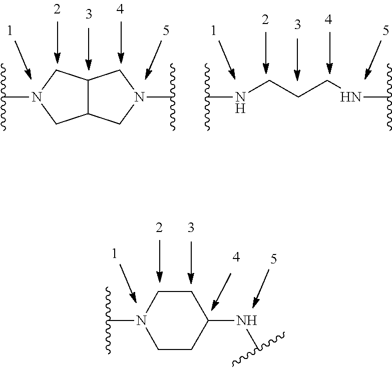

In some embodiments, the structures in Table I are not substituted with any C₁-C₆-alkyl or oxo.

In some embodiments, X³ does not comprise a ring. In some such embodiments, X⁶ is a linker selected from the group consisting of:

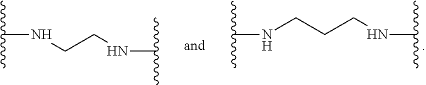 [SMILES: *NCCN* ; *NCCCN*]

Any such group is optionally substituted with one or more substituents independently selected from the group consisting of C₁-C₆-alkyl and oxo.

In some embodiments, X³ is one of the single- or double-ring structures in Table I. The ring(s) is/are optionally substituted with one or more substituents independently selected from the group consisting of halogen, hydroxy, C₁-C₆-alkyl, C₁-C₆-alkoxy, oxo, and thiocarbonyl.

In some embodiments, X³ is one of the 4- to 7-member single ring structures in Table I. The ring is optionally substituted with one or more substituents independently selected from the group consisting of halogen, hydroxy, C₁-C₆-alkyl, C₁-C₆-alkoxy, oxo, and thiocarbonyl.

In some embodiments, X³ is one of the 4- to 7-member single ring structures in Table I. The ring is optionally substituted with one or more substituents independently selected from the group consisting of halogen, hydroxy, C₁-C₆-alkyl, C₁-C₆-alkoxy, and oxo.

In some embodiments, X³ is one of the 4- to 7-member single ring structures in Table I. The ring is optionally substituted with one or more substituents independently selected from the group consisting of C₁-C₆-alkyl and oxo.

In some embodiments, X³ is:

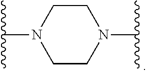 [SMILES: *N1CCN(*)CC1]

In those embodiments, the compound is encompassed by the following formula:

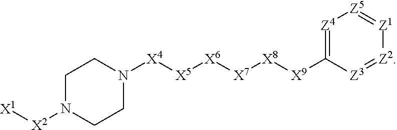

In some embodiments, X³ is:

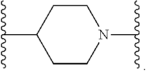 [SMILES: *C1CCN(*)CC1]

In such embodiments, the compound is encompassed by the following formula:

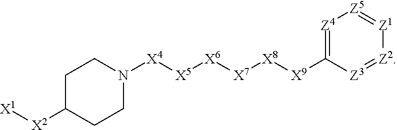

In some embodiments, X³ is:

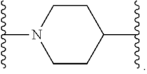 [SMILES: *C1CCN(*)CC1]

In such embodiments, the compound is encompassed by the following formula:

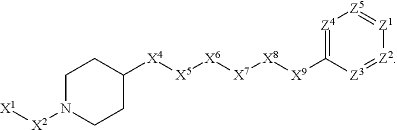

In some embodiments, X³ is:

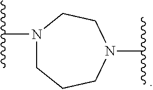 [SMILES: *N1CCCN(*)CC1]

In such embodiments, the compound is encompassed by the following formula:

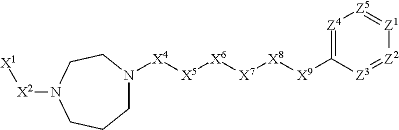

In some embodiments, X³ is:

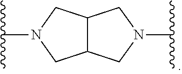 [SMILES: *N1CC2CN(*)CC2C1]

In such embodiments, the compound is encompassed by the following formula:

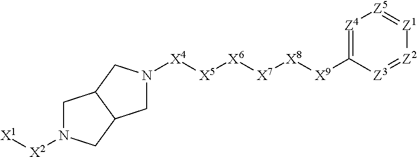

In some embodiments, X³ is:

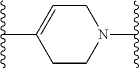 [SMILES: *C1=CCN(*)CC1]

In such embodiments, the compound is encompassed by the following formula:

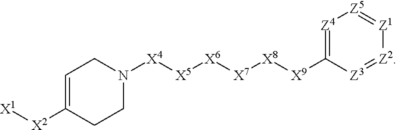

In some embodiments, one or more carbon atoms in the linker are substituted with one or two substituents independently selected from the group consisting of halogen, hydroxy, C₁-C₆-alkyl, C₁-C₆-alkoxy, oxo, and thiocarbonyl.

In some embodiments, one or more carbon atoms in the linker are substituted with one or two substituents independently selected from the group consisting of halogen, hydroxy, C₁-C₆-alkyl, C₁-C₆-alkoxy, and oxo.

In some embodiments, X³ is one of the single- or double-ring structures in Table I, and one or two of the ring atoms in the ring structure are substituted with a substituent independently selected from the group consisting of methyl and oxo. To illustrate, in some embodiments, a ring atom is substituted with an oxo substituent. The linker in such an instance may be, for example:

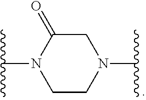 [SMILES: *N1CCN(*)C(=O)C1]

In other embodiments, for example, one or two of the ring atoms are substituted with methyl. To illustrate, the linker in such an instance may be, for example:

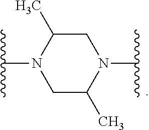 [SMILES: *N1CC(C)N(*)CC1C]

To further illustrate, the linker may alternatively be, for example:

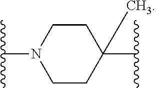 [SMILES: *N1CCC(*)(C)CC1]

### D. Preferred Embodiments of X⁴

X⁴ is selected from the group consisting of a bond, —CH₂—, —O—, —C(S)—, —C(O)—, —S(O)—, and —S(O)₂—. The —CH₂— is optionally substituted with up to two substituents independently selected from the group consisting of alkyl, alkenyl, and carbocyclyl.

In some embodiments, X⁴ is selected from the group consisting of a bond, —CH₂—, —O—, —C(S)—, —C(O)—, —S(O)—, and —S(O)₂—. The —CH₂— is optionally substituted with up to two substituents independently selected from the group consisting of C₁-C₆-alkyl, C₂-C₆-alkenyl, and C₃-C₆-carbocyclyl.

In some embodiments, X⁴ is selected from the group consisting of a bond, —CH₂—, —O—, —C(S)—, —C(O)—, —S(O)—, and —S(O)₂—. The —CH₂— is optionally substituted with up to two substituents independently selected from the group consisting of C₁-C₆-alkyl, C₂-C₆-alkenyl, and C₃-C₆-cycloalkyl.

In some embodiments, X⁴ is a single bond. In such embodiments, the compound is encompassed by the following formula:

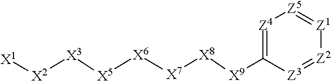

In some embodiments, X⁴ is —CH₂—. In such embodiments, the compound is encompassed by the following formula:

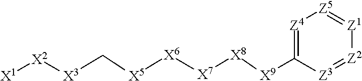

In some embodiments, X⁴ is —O—. In those embodiments, the compound is encompassed by the following formula:

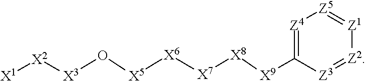

In some embodiments, X⁴ is —C(S)—. In such embodiments, the compound is encompassed by the following formula:

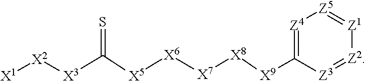

In some embodiments, X⁴ is —C(O)—. In such embodiments, the compound is encompassed by the following formula:

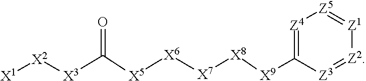

In some embodiments, X⁴ is —S(O)—. In such embodiments, the compound is encompassed by the following formula:

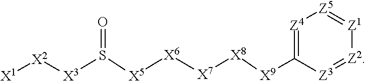

In some embodiments, X⁴ is —S(O)₂—. In such embodiments, the compound is encompassed by the following formula:

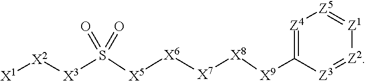

### E. Preferred Embodiments of X⁵

X⁵ is selected from the group consisting of a bond, —CH₂—, and carbocyclyl. The —CH₂— is optionally substituted with up to two substituents independently selected from the group consisting of alkyl, alkenyl, and carbocyclyl.

In some embodiments, X⁵ is selected from the group consisting of a bond, —CH₂—, and carbocyclyl. The —CH₂— is optionally substituted with up to two substituents independently selected from the group consisting of C₁-C₆-alkyl, C₂-C₆-alkenyl, and C₁-C₆-carbocyclyl.

X⁵ is selected from the group consisting of a bond and —CH₂—. The —CH₂— is optionally substituted with up to two substituents independently selected from the group consisting of alkyl, alkenyl, and carbocyclyl.

In some embodiments, X⁵ is selected from the group consisting of a bond and —CH₂—. The —CH₂— is optionally substituted with up to two substituents independently selected from the group consisting of C₁-C₆-alkyl, C₂-C₆-alkenyl, and C₁-C₆-carbocyclyl.

In some embodiments, X⁵ is a single bond. In such embodiments, the compound is encompassed by the following formula:

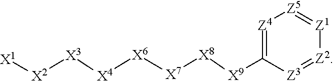

In some embodiments, X⁵ is —CH₂—. In such embodiments, the compound is encompassed by the following formula:

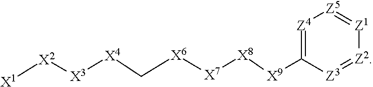

In some embodiments, X⁵ is —CH₂— substituted with up to two independently selected C₁-C₆-alkyl. For example, in some embodiments, X⁵ is —CH₂— substituted with C₁-alkyl (i.e., methyl). In such embodiments, the compound is encompassed by the following formula:

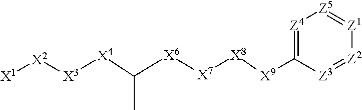

In other embodiments, X⁵ is —CH₂— substituted with two C₁-alkyl (i.e., methyl) groups. In such embodiments, the compound is encompassed by the following formula:

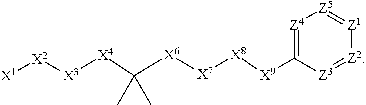

In some embodiments, X⁵ is carbocyclyl. For example, in some such embodiments, X⁵ is C₆-cycloalkyl (e.g., cyclohexyl). In such embodiments, the compound is encompassed by the following formula:

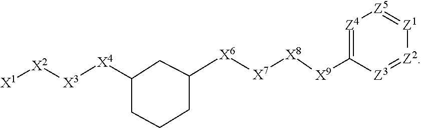

### F. Preferred Embodiments of X⁶

X⁶ is selected from the group consisting of a bond, —CH₂—, and carbocyclyl. The —CH₂— is optionally substituted with up to two substituents independently selected from the group consisting of alkyl, alkenyl, and carbocyclyl.

In some embodiments, X⁶ is selected from the group consisting of a bond, —CH₂—, and carbocyclyl. The —CH₂— is optionally substituted with up to two substituents independently selected from the group consisting of C₁-C₆-alkyl, C₂-C₆-alkenyl, and C₁-C₆-carbocyclyl.

X⁶ is selected from the group consisting of a bond and —CH₂—. The —CH₂— is optionally substituted with up to two substituents independently selected from the group consisting of alkyl, alkenyl, and carbocyclyl.

In some embodiments, X⁶ is selected from the group consisting of a bond and —CH₂—. The —CH₂— is optionally substituted with up to two substituents independently selected from the group consisting of C₁-C₆-alkyl, C₂-C₆-alkenyl, and C₁-C₆-carbocyclyl.

In some embodiments, X⁶ is a single bond. In such embodiments, the compound is encompassed by the following formula:

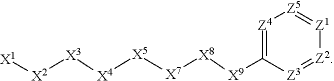

In some embodiments, X⁶ is —CH₂—. In such embodiments, the compound is encompassed by the following formula:

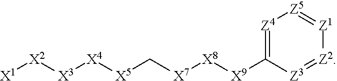

In some embodiments, X⁶ is —CH₂— substituted with up to two independently selected C₁-C₆-alkyl. For example, in some embodiments, X⁶ is —CH₂— substituted with C₁-alkyl (i.e., methyl). In such embodiments, the compound is encompassed by the following formula:

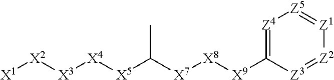

In other embodiments, X⁵ is —CH₂— substituted with two C₁-alkyl (i.e., methyl) groups. In such embodiments, the compound is encompassed by the following formula:

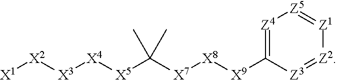

In some embodiments, X⁶ is carbocyclyl. For example, in some such embodiments, X⁶ is C₆-cycloalkyl (e.g., cyclohexyl). In such embodiments, the compound is encompassed by the following formula:

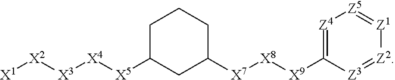

### G. Preferred Embodiments of X⁷

X⁷ is selected from the group consisting of —CH₂—, —O—, —C(O)—, —C(S)—, —S—, —S(O)—, —S(O)₂—, —NH—, —C(O)—NH—, —C(S)—NH—, —NH—C(O)—, and —NH—C(S)—. The —CH₂— is optionally substituted with up to two substituents independently selected from the group consisting of alkyl, alkenyl, and carbocyclyl. The —NH— is optionally substituted with a substituent selected from the group consisting of alkyl, alkenyl, alkynyl, alkoxyalkyl, carbocyclyl, and carbocyclylalkyl, wherein any such substituent is optionally substituted with one or more independently selected halogen.

In some embodiments, X⁷ is selected from the group consisting of —CH₂—, —O—, —C(O)—, —C(S)—, —S—, —S(O)—, —S(O)₂—, —NH—, —C(O)—NH—, —C(S)—NH—, —NH—C(O)—, and —NH—C(S)—. The —CH₂— is optionally substituted with up to two substituents independently selected from the group consisting of C₁-C₆-alkyl, C₂-C₆-alkenyl, and C₃-C₆-carbocyclyl. The —NH— is optionally substituted with a substituent selected from the group consisting of Cᵣ C₆-alkyl, C₂-C₆-alkenyl, C₂-C₆-alkynyl, C₁-C₆-alkoxy-C₁-C₆-alkyl, C₃-C₆-carbocyclyl, and C₃-C₆-carbocyclyl-C₁-C₆-alkyl, wherein any such substituent is optionally substituted with one or more independently selected halogen.

In some embodiments, X⁷ is —CH₂—. In some such embodiments, for example, X⁷ is —CH₂—. In these embodiments, the compound is encompassed by the following formula:

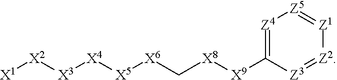

In some embodiments, X⁷ is —O—. In these embodiments, the compound is encompassed by the following formula:

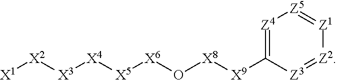

In some embodiments, X⁷ is —C(O)—. In these embodiments, the compound is encompassed by the following formula:

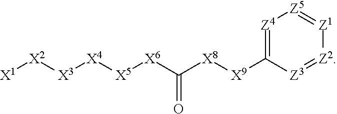

In some embodiments, X⁷ is —C(S)—. In these embodiments, the compound is encompassed by the following formula:

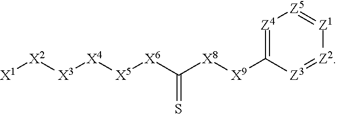

In some embodiments, X⁷ is —S—. In these embodiments, the compound is encompassed by the following formula:

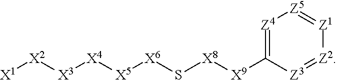

In some embodiments, X⁷ is —S(O)—. In these embodiments, the compound is encompassed by the following formula:

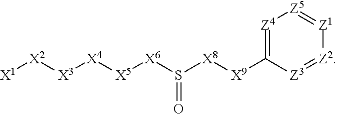

In some embodiments, X⁷ is —S(O)₂—. In these embodiments, the compound is encompassed by the following formula:

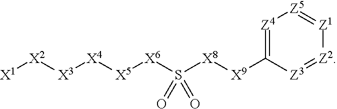

In some embodiments, X⁷ is —NH—. In these embodiments, the compound is encompassed by the following formula:

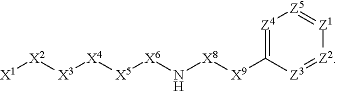

In some embodiments, X⁷ is —NH— substituted with C₁-C₆-alkyl. In some such embodiments, X⁷ is —NH— substituted with C₁-alkyl (i.e., methyl). In these embodiments, the compound is encompassed by the following formula:

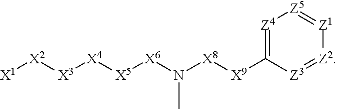

In some embodiments, X⁷ is —C(O)—NH—. In these embodiments, the compound is encompassed by the following formula:

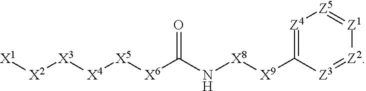

In some embodiments, X⁷ is —C(S)—NH—. In these embodiments, the compound is encompassed by the following formula:

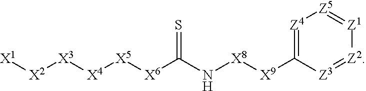

In some embodiments, X⁷ is —NH—C(O)—. In these embodiments, the compound is encompassed by the following formula:

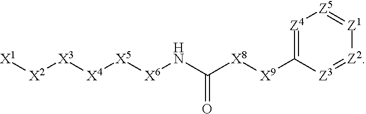

In some embodiments, X⁷ is —NH—C(O)— substituted with methyl. In these embodiments, the compound is encompassed by the following formula:

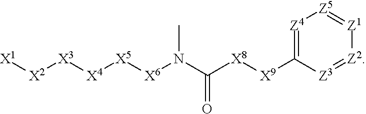

In some embodiments, X⁷ is —NH—C(S)—. In these embodiments, the compound is encompassed by the following formula:

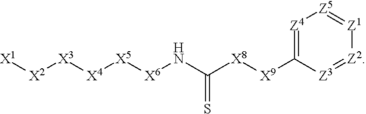

In some embodiments, X⁷ is —NH—C(S)— substituted with methyl. In these embodiments, the compound is encompassed by the following formula:

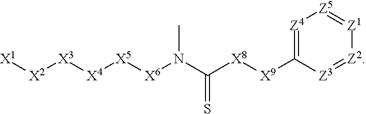

### H. Preferred Embodiments of X⁴, X⁵, X⁶, and X⁷

In some embodiments of this invention, the compound corresponds in structure to the following formula:

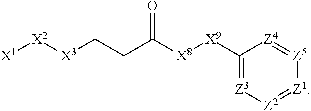

In some embodiments of this invention, the compound corresponds in structure to the following formula:

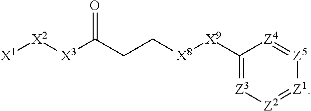

In some embodiments of this invention, the compound corresponds in structure to the following formula:

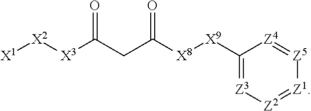

In some embodiments of this invention, the compound corresponds in structure to the following formula:

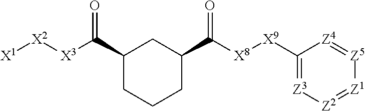

In some embodiments of this invention, the compound corresponds in structure to the following formula:

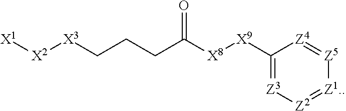

In some embodiments of this invention, the compound corresponds in structure to the following formula:

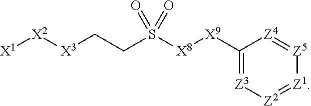

In some embodiments of this invention, the compound corresponds in structure to the following formula:

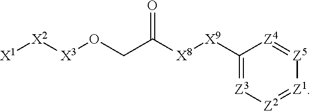

In some embodiments of this invention, the compound corresponds in structure to the following formula:

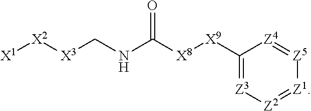

In some embodiments of this invention, the compound corresponds in structure to the following formula:

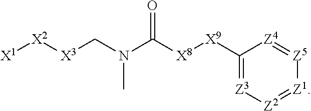

In some embodiments of this invention, the compound corresponds in structure to the following formula:

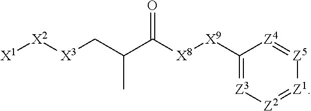

In some embodiments of this invention, the compound corresponds in structure to the following formula:

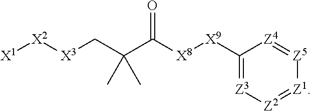

In some embodiments of this invention, the compound corresponds in structure to the following formula:

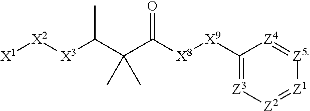

In some embodiments of this invention, the compound corresponds in structure to the following formula:

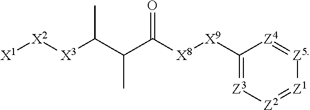

In some embodiments of this invention, the compound corresponds in structure to the following formula:

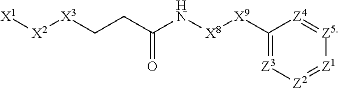

In some embodiments of this invention, the compound corresponds in structure to the following formula:

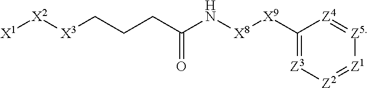

In some embodiments of this invention, the compound corresponds in structure to the following formula:

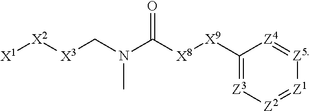

In some embodiments of this invention, the compound corresponds in structure to the following formula:

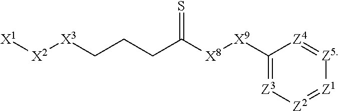

In some embodiments of this invention, the compound corresponds in structure to the following formula:

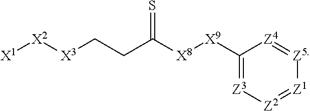

In some embodiments of this invention, the compound corresponds in structure to the following formula:

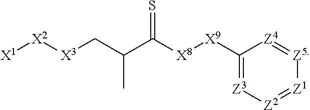

In some embodiments of this invention, the compound corresponds in structure to the following formula:

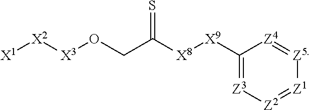

In some embodiments of this invention, the compound corresponds in structure to the following formula:

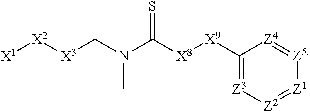

In some embodiments of this invention, the compound corresponds in structure to the following formula:

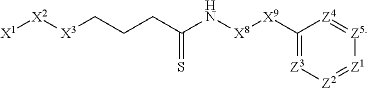

In some embodiments of this invention, the compound corresponds in structure to the following formula:

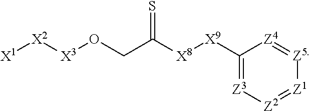

### I. Preferred Embodiments of X⁸

X⁸ is selected from the group consisting of piperidinyl, piperazinyl, homopiperazinyl, and pyrrolidinyl. The piperidinyl, piperazinyl, homopiperazinyl or pyrrolidinyl is optionally substituted with one or more independently selected alkyl.

In some embodiments, X⁸ is piperidinyl or pyrrolidinyl. The piperidinyl or pyrrolidinyl is optionally substituted with one or more independently selected alkyl.

In some embodiments, X⁸ is piperidinyl or pyrrolidinyl. The piperidinyl or pyrrolidinyl is optionally substituted with one or more independently selected C₁-C₆-alkyl.

In some embodiments, X⁸ is piperidinyl optionally substituted with one or more independently selected C₁-C₆-alkyl. To illustrate, in some such embodiments, X⁸ is piperidinyl. In some such embodiments, the compound is encompassed by the following formula:

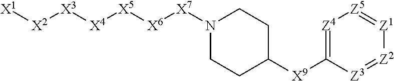

In other such embodiments, the compound is encompassed by the following formula:

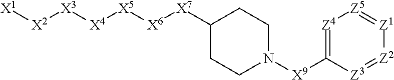

In some embodiments, X⁸ is piperidinyl optionally substituted with one or more independently selected C₁-C₆-alkyl. To illustrate, in some such embodiments, X⁸ is piperidinyl. In some such embodiments, the compound is encompassed by the following formula:

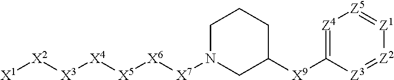

In some embodiments, X⁸ is pyrrolidinyl optionally substituted with one or more independently selected alkyl. To illustrate, in some such embodiments, X⁸ is pyrrolidinyl. In some such embodiments, the compound is encompassed by the following formula:

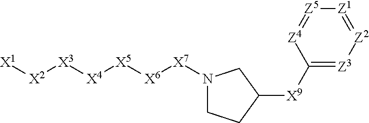

In some embodiments, X⁸ is piperazinyl optionally substituted with one or more independently selected alkyl. To illustrate, in some such embodiments, X⁸ is piperazinyl. In some such embodiments, the compound is encompassed by the following formula:

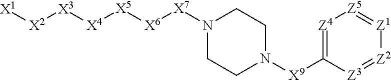

In some embodiments, X⁸ is homopiperazinyl optionally substituted with one or more independently selected alkyl. To illustrate, in some such embodiments, X⁸ is homopiperazinyl. In some such embodiments, the compound is encompassed by the following formula:

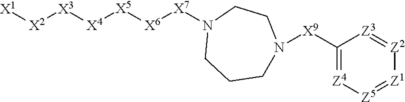

### J. Preferred Embodiments of X⁹

X⁹ is selected from the group consisting of a bond, —O—, —C(O)—, —S—, —S(O)—, —S(O)₂—, and —NH—, preferably —O—, —C(O)—, —S—, —S(O)—, —S(O)₂—, and —NH—. Here, the —NH— optionally is substituted with a substituent selected from the group consisting of alkyl, alkenyl, alkynyl, alkoxyalkyl, carbocyclyl, and carbocyclylalkyl. Any such substituent is optionally substituted with one or more independently selected halogen.

In some embodiments, X⁹ is selected from the group consisting of a bond, —O—, —C(O)—, —S—, —S(O)—, —S(O)₂—, and —NH—, preferably —O—, —C(O)—, —S—, —S(O)—, —S(O)₂—, and —NH—. Here, the —NH— optionally is substituted with a substituent selected from the group consisting of C₁-C₆-alkyl, C₂-C₆-alkenyl, C₂-C₆-alkynyl, C₁-C₆-alkoxy C₁-C₆-alkyl, C₃-C₆-carbocyclyl, and C₃-C₆-carbocyclyl-C₁-C₆-alkyl. Any such substituent is optionally substituted with one or more independently selected halogen.

In some embodiments X⁹ is different from a bond.

In some embodiments, X⁹ is —NH— optionally substituted with a substituent selected from the group consisting of C₁-C₆-alkyl, C₂-C₆-alkenyl, C₂-C₆-alkynyl, C₁-C₆-alkoxy C₁-C₆-alkyl, C₃-C₆-carbocyclyl, and C₃-C₆-carbocyclyl-C₁-C₆-alkyl. Any such substituent is optionally substituted with one or more independently selected halogen. To illustrate, in some such embodiments, X¹ is —NH—. In such embodiments, the compound is encompassed by the following formula:

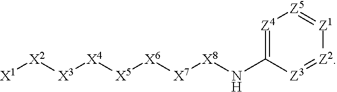

In other such embodiments, the compound is encompassed by the following formula:

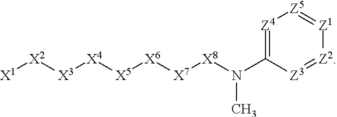

In some embodiments, for example, X⁹ is a single bond. Here, the compound is encompassed by the following formula:

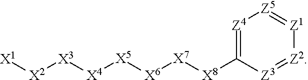

In some embodiments, X⁹ is —O—. In such embodiments, the compound is encompassed by the following formula:

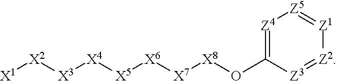

In some embodiments, X⁹ is —C(O)—. In such embodiments, the compound is encompassed by the following formula:

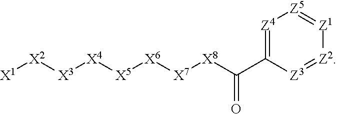

In some embodiments, X⁹ is —S—. In such embodiments, the compound is encompassed by the following formula:

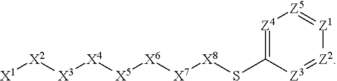

In some embodiments, X⁹ is —S(O)—. In such embodiments, the compound is encompassed by the following formula:

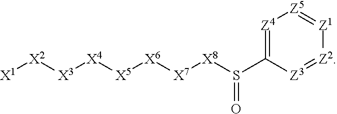

In some embodiments, X⁹ is —S(O)₂—. In such embodiments, the compound is encompassed by the following formula:

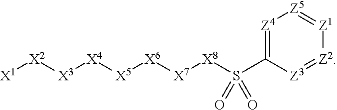

### K. Preferred Embodiments of Z¹, Z², Z³, Z⁴, and Z⁵

Z¹ is selected from the group consisting of N and CH. The CH is optionally substituted with a substituent selected from the group consisting of halogen, nitro, cyano, aminosulfonyl, alkyl, alkoxy, alkoxycarbonyl, alkylsulfanyl, alkylsulfinyl, alkylsulfonyl, aryl, arylsulfanyl, arylsulfinyl, arylsulfonyl, heteroaryl, heteroarylsulfanyl, heteroarylsulfinyl, and heteroarylsulfonyl. The alkyl, alkoxy, alkoxycarbonyl, alkylsulfanyl, alkylsulfinyl, alkylsulfonyl, aryl, arylsulfanyl, arylsulfinyl, arylsulfonyl, heteroaryl, heteroarylsulfanyl, heteroarylsulfinyl, and heteroarylsulfonyl are optionally substituted with one or more substituents independently selected from the group consisting of halogen and alkyl. The aminosulfonyl is optionally substituted with up to two independently selected alkyl.

In some embodiments, Z¹ is selected from the group consisting of N and CH. The CH is optionally substituted with a substituent selected from the group consisting of halogen, nitro, cyano, aminosulfonyl, C₁-C₆-alkyl, C₁-C₆-alkoxy, C₁-C₆-alkoxycarbonyl, C₁-C₆-alkylsulfanyl, C₁-C₆-alkylsulfinyl, C₁-C₆-alkylsulfonyl, aryl, arylsulfanyl, arylsulfinyl, arylsulfonyl, heteroaryl, heteroarylsulfanyl, heteroarylsulfinyl, and heteroarylsulfonyl. The C₁-C₆-alkyl, C₁-C₆-alkoxy, C₁-C₆-alkoxycarbonyl, C₁-C₆-C₁-C₆-alkylsulfanyl, C₁-C₆-alkylsulfinyl, C₁-C₆-alkylsulfonyl, aryl, arylsulfanyl, arylsulfinyl, arylsulfonyl, heteroaryl, heteroarylsulfanyl, heteroarylsulfinyl, and heteroarylsulfonyl are optionally substituted with one or more substituents independently selected from the group consisting of halogen and C₁-C₆-alkyl. The aminosulfonyl is optionally substituted with up to two independently selected C₁-C₆-alkyl.

In some embodiments, Z¹ is N. Such embodiments are encompassed by the following structure:

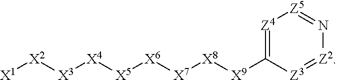

In some embodiments, Z¹ is optionally substituted CH. In some such embodiments, for example, Z¹ is CH. Such embodiments are encompassed by the following structure:

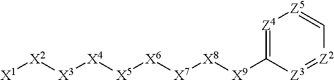

In other embodiments, Z¹ is CH substituted with a substituent selected from the group consisting of alkylsulfonyl, alkoxy, cyano, haloalkyl, halogen, nitro, haloarylsulfonyl, haloalkylsulfanyl, haloalkoxy, alkoxycarbonyl, 5-membered heteroaryl, alkylsulfanyl, alkylsulfinyl, and dialkylaminosulfonyl, wherein the 5-membered heteroaryl optionally is substituted with C₁-C₆-alkyl.

In some embodiments, Z¹ is CH substituted with an electron-withdrawing substituent. Such substituents include, for example, halogen, nitro, cyano, halo-C₁-C₆-alkyl, halo-C₁-C₆-alkoxy, and halo-C₁-C₆-alkylsulfanyl, alkylsulfinyl, alkylsulfonyl, and dialkylaminosulfonyl.

In some embodiments, Z¹ is CH substituted with a halogen. For example, in some such embodiments, Z¹ is CH substituted with chloro. These embodiments are encompassed by the following structure:

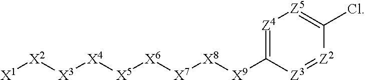

In some embodiments, Z¹ is CH substituted with nitro. Such embodiments are encompassed by the following structure:

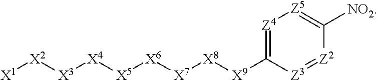

In some embodiments, Z¹ is CH substituted with cyano. Such embodiments are encompassed by the following structure:

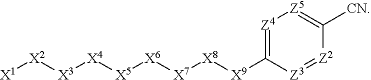

In some embodiments, Z¹ is CH substituted with halo-C₁-C₆-alkyl. For example, in some such embodiments, Z¹ is CH substituted with trifluoro-C₁-alkyl (i.e., trifluoromethyl). Such embodiments are encompassed by the following structure:

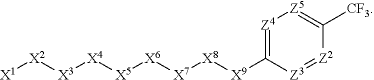

In some embodiments, Z¹ is CH substituted with C₁-C₆-alkoxy. For example, in some such embodiments, Z¹ is CH substituted with C₁-alkoxy (i.e., methoxy). Such embodiments are encompassed by the following structure:

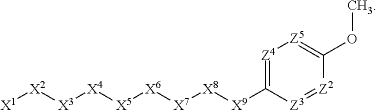

In some embodiments, Z¹ is CH substituted with C₁-C₆-alkylsulfanyl. For example, in some such embodiments, Z¹ is CH substituted with C₁-alkylsulfanyl (i.e., methylsulfinyl). Such embodiments are encompassed by the following structure:

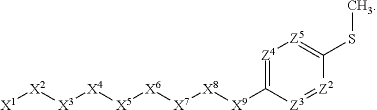

In some embodiments, Z¹ is CH substituted with halo-C₁-C₆-alkoxy. For example, in some such embodiments, Z¹ is CH substituted with fluoro-C₁-alkoxy. Such embodiments are encompassed by the following structure:

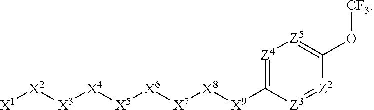

In some embodiments, Z¹ is CH substituted with halo-C₁-C₆-alkylsulfanyl. For example, in some such embodiments, Z¹ is CH substituted with fluoro-C₁-alkylsulfanyl. Such embodiments are encompassed by the following structure:

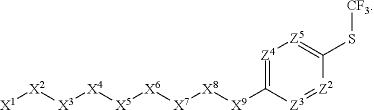

In some embodiments, Z¹ is CH substituted with C₁-C₆-alkylsulfinyl. For example, in some such embodiments, Z¹ is CH substituted with C₁-alkylsulfinyl (i.e., methylsulfinyl). Such embodiments are encompassed by the following structure:

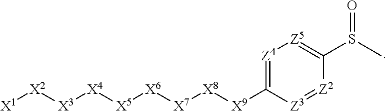

In some embodiments, Z¹ is CH substituted with C₁-C₆-alkylsulfonyl. For example, in some such embodiments, Z¹ is CH substituted with C₁-alkylsulfonyl (i.e., methylsulfonyl). Such embodiments are encompassed by the following structure:

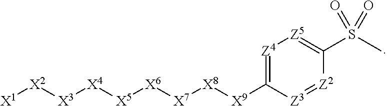

In some embodiments, Z¹ is CH substituted with di-C₁-C₆-alkylaminosulfonyl. For example, in some such embodiments, Z¹ is CH substituted with di-C₁-alkylaminosulfonyl (i.e., dimethylaminosulfonyl). Such embodiments are encompassed by the following structure:

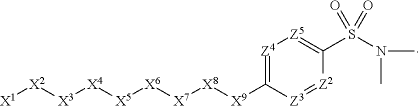

In some embodiments, Z¹ is CH substituted with haloarylsulfonyl. For example, in some such embodiments, Z¹ is CH substituted with 4-fluoro-phenyl-sulfonyl. Such embodiments are encompassed by the following structure:

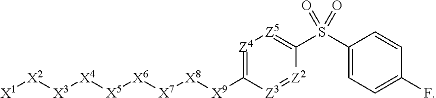

In some embodiments, Z¹ is CH substituted with C₁-C₆-alkoxycarbonyl. For example, in some such embodiments, Z¹ is CH substituted with C₂-alkoxycarbonyl (i.e., ethoxycarbonyl). Such embodiments are encompassed by the following structure:

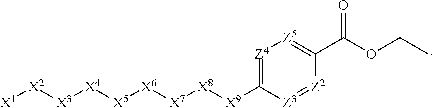

In some embodiments, Z¹ is CH substituted with heteroaryl optionally substituted with C₁-C₆-alkyl. For example, in some such embodiments, Z¹ is CH substituted with methyltetrazoyl). And is encompassed by the following structure:

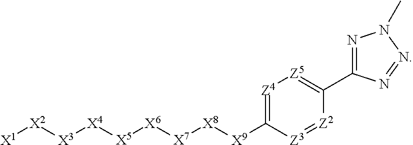

Z² is selected from the group consisting of N and CH. The CH is optionally substituted with a substituent selected from the group consisting of cyano, halogen, nitro, alkyl, alkoxy, haloalkyl, and haloalkylsulfanyl.

In some embodiments, Z² is selected from the group consisting of N and CH. The CH is optionally substituted with a substituent selected from the group consisting of cyano, halogen, nitro, C₁-C₆-alkyl, C₁-C₆-alkoxy, halo-C₁-C₆-alkyl, halo-C₁-C₆-sulfanyl.

In some embodiments, Z² is selected from the group consisting of N and CH. The CH is optionally substituted with a substituent selected from the group consisting of cyano, halogen, C₁-C₆-alkyl, C₁-C₆-alkoxy, halo-C₁-C₆-alkyl, halo-C₁-C₆-sulfanyl.

In some embodiments, Z² is N. Such embodiments are encompassed by the following structure:

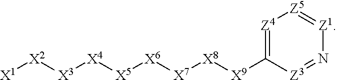

In some embodiments, Z² is CH substituted with a substituent selected from the group consisting of cyano, halogen, nitro, C₁-C₆-alkyl, C₁-C₆-alkoxy, halo-C₁-C₆-alkyl, and halo-C₁-C₆-alkylsulfanyl. In some such embodiments, for example, Z² is CH. Such embodiments are encompassed by the following structure:

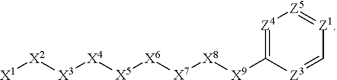

In some embodiments, Z² is CH substituted with halo-C₁-C₆-alkyl. For example, in some such embodiments, Z² is CH substituted with fluoro-C₁— C₆-alkyl. To illustrate, Z² can be, for example, CH substituted with trifluoromethyl such that the compound is encompassed by the following structure:

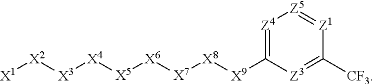

In some embodiments, Z² is CH substituted with cyano. Such embodiments are encompassed by the following structure:

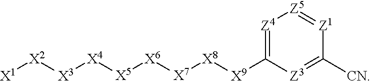

In some embodiments, Z² is CH substituted with halogen. For example, in some such embodiments, Z² is CH substituted with chloro. These embodiments are encompassed by the following structure:

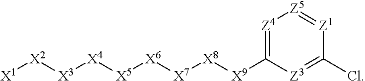

In some embodiments, Z² is CH substituted with C₁-C₆-alkyl. For example, in some such embodiments, Z² is CH substituted with C₁-alkyl (i.e., methyl). Such embodiments are encompassed by the following structure:

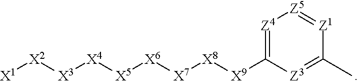

In some embodiments, Z² is CH substituted with C₁-C₆-alkoxy. For example, in some such embodiments, Z² is CH substituted with C₄-alkoxy (e.g., isobutoxy). Such embodiments are encompassed by the following structure:

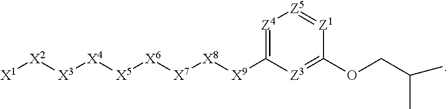

In other such embodiments, Z² is CH substituted with C₂-alkoxy (e.g., ethoxy). Such embodiments are encompassed by the following structure:

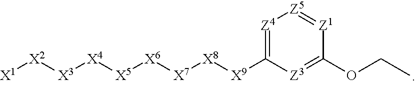

In yet other such embodiments, Z² is CH substituted with C₁-alkoxy (e.g., methoxy). Such embodiments are encompassed by the following structure:

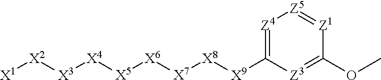

In some embodiments, Z² is CH substituted with halo-C₁-C₆-alkylsulfanyl. For example, in some such embodiments, Z² is CH substituted with fluoro-C₁-C₆-alkylsulfanyl (e.g., trifluoromethylsulfanyl). Such embodiments are encompassed by the following structure:

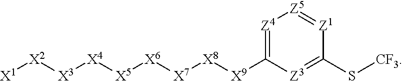

Each of Z³, Z⁴, and Z⁵ is independently selected from the group consisting of N and CH. The CH is optionally substituted with a substituent selected from the group consisting of halogen, cyano, nitro, alkyl, alkoxy, alkylsulfanyl, haloalkyl, haloalkoxy, and haloalkylsulfanyl.

In some embodiments, each of Z³, Z⁴, and Z⁵ is independently selected from the group consisting of N and CH. The CH is optionally substituted with a substituent selected from the group consisting of halogen, cyano, nitro, C₁-C₆-alkyl, C₁-C₆-alkoxy, C₁-C₆-alkylsulfanyl, halo-C₁-C₆-alkyl, halo-C₁-C₆-alkoxy, and halo-C₁-C₆-alkylsulfanyl.

In some embodiments, Z³ is halo-C₁-C₆-alkyl. For example, in some such embodiments, Z³ is trifluoromethyl. Such embodiments are encompassed by the following structure:

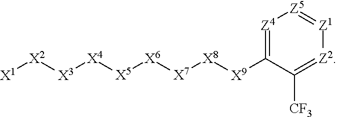

In some embodiments, Z², Z³, Z⁴, and Z⁵ are each CH. Such embodiments are encompassed by the following structure:

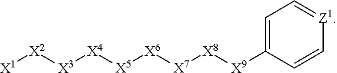

In some embodiments, Z¹, Z³, Z⁴, and Z⁵ are each CH. Such embodiments are encompassed by the following structure:

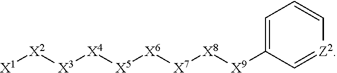

In some embodiments, Z², Z⁴, and Z⁵ are each CH. Such embodiments are encompassed by the following structure:

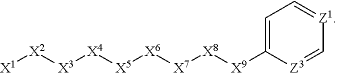

In some embodiments, Z², Z⁴, and Z⁵ are each CH and Z³ is N. Such embodiments are encompassed by the following structure:

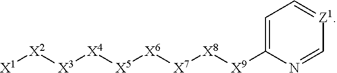

In some embodiments, Z³, Z⁴, and Z⁵ are each CH and Z¹ is N. Such embodiments are encompassed by the following structure:

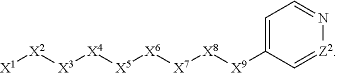

In some embodiments, Z¹, Z³, and Z⁴ are each CH and Z² is N. Such embodiments are encompassed by the following structure:

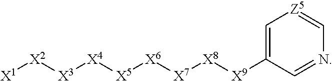

In some embodiments, Z², Z⁴, and Z⁵ are each CH and Z⁵ is N. Such embodiments are encompassed by the following structure:

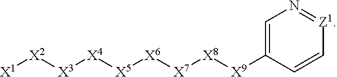

In some embodiments, Z² and Z⁴ are each CH and Z³ is N. Such embodiments are encompassed by the following structure:

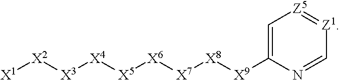

### L. Preferred Embodiments of Z¹, Z², Z³, Z⁴, and Z⁵

In some embodiments, none of Z¹, Z², Z³, Z⁴, and Z⁵ are N. In some such embodiments, Z¹, Z², Z³, Z⁴, and Z⁵ together with the atom to which they are bonded form a 6-membered ring, wherein only one of Z¹, Z², Z³, Z⁴, and Z⁵ is substituted CH. Table II shows examples of such groups.

TABLE II
Example of Z¹, Z², Z³, Z⁴, and Z⁵

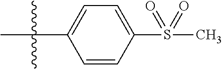 [SMILES: *c1ccc(S(C)(=O)=O)cc1]

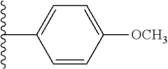 [SMILES: *c1ccc(OC)cc1]

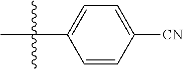 [SMILES: *c1ccc(C#N)cc1]

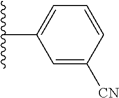 [SMILES: *c1cccc(C#N)c1]

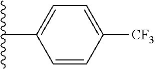 [SMILES: *c1ccc(C(F)(F)F)cc1]

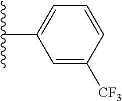 [SMILES: *c1cccc(C(F)(F)F)c1]

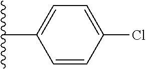 [SMILES: *c1ccc(Cl)cc1]

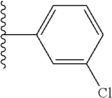 [SMILES: *c1cccc(Cl)c1]

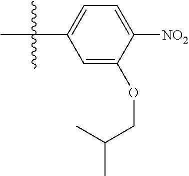 [SMILES: *c1ccc([N+](=O)[O-])c(OCC(C)C)c1]

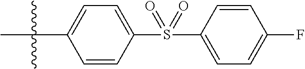 [SMILES: *c1ccc(S(=O)(=O)c2ccc(F)cc2)cc1]

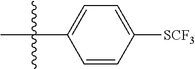

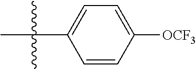 [SMILES: *c1ccc(OC(F)(F)F)cc1]

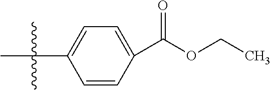 [SMILES: *c1ccc(C(=O)OCC)cc1]

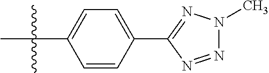 [SMILES: *c1ccc(-c2nnn(C)n2)cc1]

In other such embodiments, only two of Z¹, Z², Z³, Z⁴, and Z⁵ are substituted CH. Table III shows examples of such groups:

TABLE III
Example of Z¹, Z², Z³, Z⁴, and Z⁵

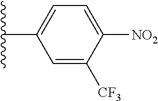 [SMILES: *c1ccc([N+](=O)[O-])c(C(F)(F)F)c1]

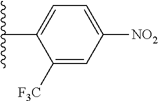 [SMILES: *c1ccc([N+](=O)[O-])cc1C(F)(F)F]

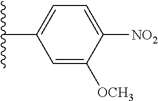 [SMILES: *c1ccc([N+](=O)[O-])c(OC)c1]

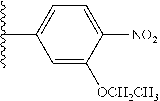 [SMILES: *c1ccc([N+](=O)[O-])c(OCC)c1]

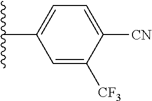 [SMILES: *c1ccc(C#N)c(C(F)(F)F)c1]

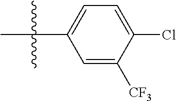 [SMILES: *c1ccc(Cl)c(C(F)(F)F)c1]

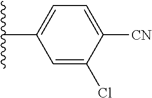 [SMILES: *c1ccc(C#N)c(Cl)c1]

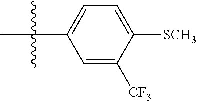 [SMILES: *c1ccc(SC)c(C(F)(F)F)c1]

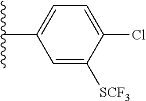

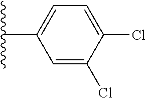 [SMILES: *c1ccc(Cl)c(Cl)c1]

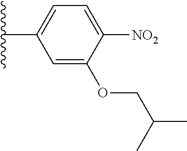 [SMILES: *c1ccc([N+](=O)[O-])c(OCC(C)C)c1]

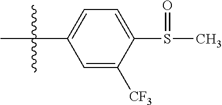 [SMILES: *c1ccc(S(C)=O)c(C(F)(F)F)c1]

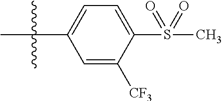 [SMILES: *c1ccc(S(C)(=O)=O)c(C(F)(F)F)c1]

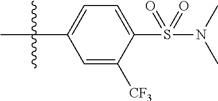 [SMILES: *c1ccc(S(=O)(=O)N(C)C)c(C(F)(F)F)c1]

In some embodiments, at least one of Z¹, Z², Z³, Z⁴, and Z⁵ is N.

In some embodiments, two of Z¹, Z², Z³, Z⁴, and Z⁵ are each N. In other embodiments, only one of Z¹, Z², Z³, Z⁴, and Z⁵ is N. Table IV shows examples of such groups.

TABLE IV
Example of Z¹, Z², Z³, Z⁴, and Z⁵

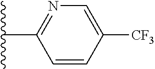 [SMILES: *c1ccc(C(F)(F)F)cn1]

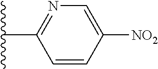 [SMILES: *c1ccc([N+](=O)[O-])cn1]

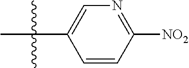 [SMILES: *c1ccc([N+](=O)[O-])nc1]

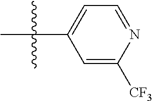 [SMILES: *c1ccnc(C(F)(F)F)c1]

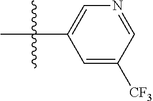 [SMILES: *c1cncc(C(F)(F)F)c1]

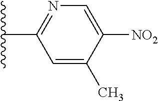 [SMILES: *c1cc(C)c([N+](=O)[O-])cn1]

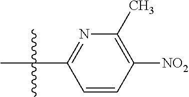 [SMILES: *c1ccc([N+](=O)[O-])c(C)n1]

### M. Examples of Various Specific Preferred Embodiments

In some embodiments of this invention, the compound is defined as corresponding in structure to the following formula:

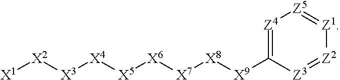

In some such embodiments,
X¹ is selected from the group consisting of phenyl, 5-member heteroaryl, 6-member heteroaryl and alkyl wherein:
   the 5-member heteroaryl is substituted with haloalkyl;
   the phenyl and 6-member heteroaryl are optionally substituted with one or more substituents independently selected from the group consisting of alkyl, haloalkyl, halogen, alkoxy, haloalkoxy, phenylalkoxy, aryl, cyano and phenoxy wherein:
      the phenylalkoxy are optionally substituted with one or more haloalkyl; and
X² is selected from the group consisting of a bond, —CH₂—O—, —C(O)—, —N(H)— and —C(S)—;
X³ is selected from the group consisting of

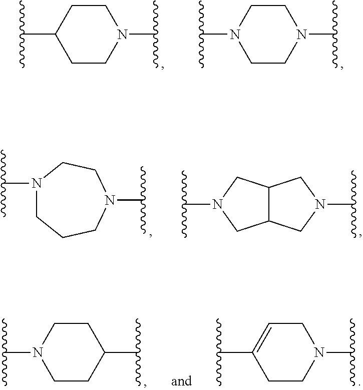

X⁴ is selected from the group consisting of a bond, —CH₂—, —O—, and —C(O)—, wherein the —CH₂— is optionally substituted with up to two independently selected alkyl;
X⁵ is selected from the group consisting of a bond and —CH₂—;
X⁶ is selected from the group consisting of a bond, —CH₂— and cycloalkyl wherein the —CH₂— is optionally substituted with up to two independently selected alkyl;
X⁷ is selected from the group consisting of —C(O)—, —C(S)—, —NH—C(O)—, —C(O)—NH—, —C(S)—NH—, —S(O)₂— and —C(O)—NH— wherein:
   the —NH—C(O)— and —NH—C(S)— are optionally substituted with alkyl;
X⁸ is selected from the group consisting of piperidinyl, piperazinyl, homopiperazinyl, and pyrrolidinyl;
Z¹ is selected from the group consisting of N and CH, wherein:
   the CH is optionally substituted with a substituent selected from the group consisting of nitro, halogen, cyano, alkyl, alkoxy, alkylsulfanyl, alkylsulfinyl, alkylsulfonyl, arylsulfonyl, heteroaryl, aminosulfonyl and alkoxycarbonyl wherein:
      the alkyl, alkoxy, alkylsulfanyl, arylsulfonyl, heteroaryl and aminosulfonyl are optionally substituted with one or more substituents independently selected from the group consisting of halogen and alkyl;
Z² is selected from the group consisting of N and CH, wherein:
   the CH is optionally substituted with a substituent selected from the group consisting of alkyl, halogen, cyano, alkoxy, haloalkyl, and haloalkylsulfanyl;
Z³, Z⁴, and Z⁵ are independently selected from the group consisting of N and CH.

In some embodiments of this invention, the compound is defined as corresponding in structure to the following formula:

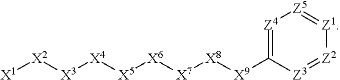

In some such embodiments,
X¹ is selected from the group consisting of phenyl, 5-member heteroaryl, 6-member heteroaryl and alkyl wherein:
   the 5-member heteroaryl is substituted with haloalkyl;
   the phenyl and 6-member heteroaryl are optionally substituted with one or more substituents independently selected from the group consisting of alkyl, haloalkyl, halogen, alkoxy, haloalkoxy, phenylalkoxy, aryl, cyano and phenoxy wherein:
      the phenylalkoxy are optionally substituted with one or more haloalkyl; and
X² is selected from the group consisting of a bond, —CH₂—O—, —C(O)—, —N(H)— and —C(S)—;
X³ is selected from the group consisting of

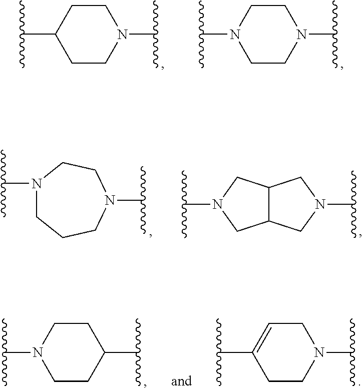

X⁴ is selected from the group consisting of a bond, —CH₂—, —O—, and —C(O)—, wherein the —CH₂— is optionally substituted with up to two independently selected alkyl;
X⁵ is selected from the group consisting of a bond and —CH₂—;
X⁶ is selected from the group consisting of a bond, —CH₂— and cycloalkyl wherein the —CH₂— is optionally substituted with up to two independently selected alkyl;
X⁷ is selected from the group consisting of —C(O)—, —C(S)—, —NH—C(O)—, —C(O)—NH—, —C(S)—NH—, —S(O)₂— and —C(O)—NH— wherein:
   the —NH—C(O)— and —NH—C(S)— are optionally substituted with alkyl;
X⁸ is piperidinyl or pyrrolidinyl;
Z¹ is selected from the group consisting of N and CH, wherein:
   the CH is optionally substituted with a substituent selected from the group consisting of nitro, halogen, cyano, alkyl, alkoxy, alkylsulfanyl, alkylsulfinyl, alkylsulfonyl, arylsulfonyl, heteroaryl, aminosulfonyl and alkoxycarbonyl wherein:
      the alkyl, alkoxy, alkylsulfanyl, arylsulfonyl, heteroaryl and aminosulfonyl are optionally substituted with one or more substituents independently selected from the group consisting of halogen and alkyl; Z² is selected from the group consisting of N and CH, wherein:
   the CH is optionally substituted with a substituent selected from the group consisting of alkyl, halogen, cyano, alkoxy, haloalkyl, and haloalkylsulfanyl;
Z³, Z⁴, and Z⁵ are independently selected from the group consisting of N and CH.

In some embodiments of this invention, the compound is defined as corresponding in structure to the following formula:

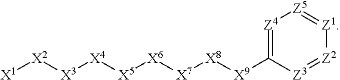

In some such embodiments,

the compound has no mirror-symmetry plane.

In some such embodiments,
X⁹ is selected from the group consisting of —O—, —C(O)—, —S—, —S(O)—, —S(O)₂—, and —NH—, wherein the —NH— optionally is substituted with a substituent selected from the group consisting of alkyl, alkenyl, alkynyl, alkoxyalkyl, carbocyclyl, and carbocyclylalkyl, wherein any such substituent is optionally substituted with one or more independently selected halogen.
   In some such embodiments,
X⁹ is selected from the group consisting of —O—, —C(O)—, —S—, —S(O)—, —S(O)₂—, and —NH—, wherein the —NH— optionally is substituted with a substituent selected from the group consisting of alkyl, alkenyl, alkynyl, alkoxyalkyl, carbocyclyl, and carbocyclylalkyl, wherein any such substituent is optionally substituted with one or more independently selected halogen, and the compound has no mirror-symmetry plane.
   In some such embodiments,
at least one of X⁴, X⁵, X⁶ is different from a bond and from —CH₂—, or X⁷ is different from —CH₂—.
   In some such embodiments,
at least one of X⁴, X⁵, X⁶ is different from a bond and from —CH₂—, or X⁷ is different from —CH₂—, and the compound has no mirror-symmetry plane.
   In some such embodiments,
X¹ is selected from the group consisting of phenyl, 5-member heteroaryl, 6-member heteroaryl and C₃-C₆-alkyl wherein:
   the 5-member heteroaryl is optionally substituted by one or more alkyl wherein:
      the alkyl is optionally substituted with one or more independently selected halogen,
   the phenyl and 6-member heteroaryl are optionally substituted at the meta and para positions by one or more substituents selected from the group consisting of alkyl, halogen, alkoxy, arylalkoxy, aryl, cyano and aryloxy wherein:
      the alkyl and alkoxy are optionally substituted with one or more independently selected halogen;
      the arylalkoxy is optionally substituted with one or more haloalkyl; and
   the phenyl is optionally substituted at the ortho positions with one or two independently selected halogen;
X² is selected from the group consisting of a bond, —CH₂—O—, —C(O)—, —N(H)— and —C(S)—;
X⁴ is selected from the group consisting of a bond, —CH₂—, —O—, and —C(O)—, wherein:
   the —CH₂— is optionally substituted with up to two independently selected alkyl;
X⁵ is selected from the group consisting of a bond and —CH₂—;
X⁶ is selected from the group consisting of a bond, —CH₂— and cycloalkyl wherein:
   the —CH₂— is optionally substituted with up to two independently selected alkyl;
X⁷ is selected from the group consisting of —C(O)—, —C(S)—, —NH—C(O)—, —C(O)—NH—, —C(S)—NH—, —S(O)₂— and —C(O)—NH— wherein:
   the —NH—C(O)— and —NH—C(S)— are optionally substituted with alkyl;
X⁸ is piperidinyl or pyrrolidinyl;
X⁹ is selected from the group consisting of —O—, —C(O)—, —S—, —S(O)—, —S(O)₂—, and —NH—, wherein the —NH— optionally is substituted with a substituent selected from the group consisting of alkyl, alkenyl, alkynyl, alkoxyalkyl, carbocyclyl, and carbocyclylalkyl, wherein any such substituent is optionally substituted with one or more independently selected halogen,
Z¹ is selected from the group consisting of N and CH, wherein:
   the CH is optionally substituted with a substituent selected from the group consisting of nitro, halogen, cyano, alkyl, alkoxy, alkylsulfanyl, alkylsulfinyl, alkylsulfonyl, arylsulfonyl, heteroaryl, aminosulfonyl and alkoxycarbonyl wherein:
      the alkyl, alkoxy, alkylsulfanyl, arylsulfonyl, heteroaryl and aminosulfonyl are optionally substituted with one or more substituents independently selected from the group consisting of halogen and alkyl;
Z² is selected from the group consisting of N and CH, wherein:
   the CH is optionally substituted with a substituent selected from the group consisting of alkyl, halogen, cyano, alkoxy, haloalkyl, alkylsulfanyl and haloalkylsulfanyl;
Z³ and Z⁴ are independently selected from the group consisting of N and CH; and Z⁵ is CH.
   In some such embodiments,
X¹ is selected from the group consisting of phenyl, 5-member heteroaryl, 6-member heteroaryl and C₃-C₆-alkyl wherein:
   the 5-member heteroaryl is optionally substituted by one or more alkyl wherein:
      the alkyl is optionally substituted with one or more independently selected halogen,
   the phenyl and 6-member heteroaryl are optionally substituted at the meta and para positions by one or more substituents selected from the group consisting of alkyl, halogen, alkoxy, arylalkoxy, aryl, cyano and aryloxy wherein:
      the alkyl and alkoxy are optionally substituted with one or more independently selected halogen;
      the arylalkoxy is optionally substituted with one or more haloalkyl; and
   the phenyl is optionally substituted at the ortho positions with one or two independently selected halogen;
X² is selected from the group consisting of a bond, —CH₂—O—, —C(O)—, —N(H)— and —C(S)—;
X⁴ is selected from the group consisting of a bond, —CH₂—, —O—, and —C(O)—, wherein:
   the —CH₂— is optionally substituted with up to two independently selected alkyl;
X⁵ is selected from the group consisting of a bond and —CH₂—;
X⁶ is selected from the group consisting of a bond, —CH₂— and cycloalkyl wherein:
   the —CH₂— is optionally substituted with up to two independently selected alkyl;
X⁷ is selected from the group consisting of —C(O)—, —C(S)—, —NH—C(O)—, —C(O)—NH—, —C(S)—NH—, —S(O)₂— and —C(O)—NH— wherein:
   the —NH—C(O)— and —NH—C(S)— are optionally substituted with alkyl;
X⁸ is piperidinyl or pyrrolidinyl;
X⁹ is selected from the group consisting of —O—, —C(O)—, —S—, —S(O)—, —S(O)₂—, and —NH—, wherein the —NH— optionally is substituted with a substituent selected from the group consisting of alkyl, alkenyl, alkynyl, alkoxyalkyl, carbocyclyl, and carbocyclylalkyl, wherein any such substituent is optionally substituted with one or more independently selected halogen,
Z¹ is selected from the group consisting of N and CH, wherein:
   the CH is optionally substituted with a substituent selected from the group consisting of nitro, halogen, cyano, alkyl, alkoxy, alkylsulfanyl, alkylsulfinyl, alkylsulfonyl, arylsulfonyl, heteroaryl, aminosulfonyl and alkoxycarbonyl wherein:
      the alkyl, alkoxy, alkylsulfanyl, arylsulfonyl, heteroaryl and aminosulfonyl are optionally substituted with one or more substituents independently selected from the group consisting of halogen and alkyl; Z² is selected from the group consisting of N and CH, wherein:
   the CH is optionally substituted with a substituent selected from the group consisting of alkyl, halogen, cyano, alkoxy, haloalkyl, alkylsulfanyl and haloalkylsulfanyl;
Z³ and Z⁴ are independently selected from the group consisting of N and CH; and
Z⁵ is CH, and the compound has no mirror-symmetry plane.
   In some such embodiments,
X¹ is selected from the group consisting of phenyl, pyridyl and thiadiazoyl, substituted by halogen, (C₁-C₆)alkyl, (C₁-C₆)alkyloxy, (C₁-C₆)haloalkyl, (C₁-C₆)haloalkyloxy, phenyloxy, halophenyloxy, benzyloxy and halobenzyloxy, preferably (C₁-C₆)alkyl, (C₁-C₆)alkyloxy, (C₁-C₆)haloalkyl, (C₁-C₆)haloalkyloxy,
X² is a bond,
X³ is piperazinyl,
X⁴ is —CH₂—,
X⁵ is selected from the group consisting of is —CH₂— and —CH(C₁-C₆)alkyl,
X⁶ is selected from the group consisting of —CH₂— and a bond,
X⁷ is CO or CS,
X⁸ is piperidinyl,
X⁹ is NH or S, preferably NH,
Z¹ is selected from the group consisting of C—NO₂, C—CN, C—S—(C₁-C₆)alkyl and C—S—(C₁-C₆)haloalkyl, preferably C—NO₂ or C—CN,
Z² is C—CF₃ or CH,
Z³ is CH or N,
Z⁴ is CH, and
Z⁵ is CH.
   In some such embodiments,
X¹ is selected from the group consisting of phenyl, pyridyl and thiadiazoyl, substituted by halogen, (C₁-C₆)alkyl, (C₁-C₆)alkyloxy, (C₁-C₆)haloalkyl, (C₁-C₆)haloalkyloxy, phenyloxy, halophenyloxy, benzyloxy and halobenzyloxy, preferably (C₁-C₆)alkyl, (C₁-C₆)alkyloxy, (C₁-C₆)haloalkyl, (C₁-C₆)haloalkyloxy,
X² is a bond,
X³ is piperazinyl,
X⁴ is —CH₂—,
X⁵ is selected from the group consisting of is —CH₂— and —CH(C₁-C₆)alkyl,
X⁶ is selected from the group consisting of —CH₂— and a bond,
X⁷ is CO or CS,
X⁸ is piperidinyl,
X⁹ is NH or S, preferably NH,
Z¹ is selected from the group consisting of C—NO₂, C—CN, C—S—(C₁-C₆)alkyl and C—S—(C₁-C₆)haloalkyl, preferably C—NO₂ or C—CN,
Z² is C—CF₃ or CH,
Z³ is CH or N,
Z⁴ is CH, and
Z⁵ is CH, and the compound has no mirror-symmetry plane.

In some embodiments of this invention, the compound is defined as corresponding in structure to the following formula:

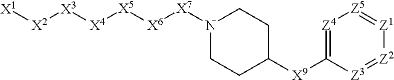

In some such embodiments,

the compound has no mirror-symmetry plane.

In some such embodiments,
X⁹ is selected from the group consisting of —O—, —C(O)—, —S—, —S(O)—, —S(O)₂—, and —NH—, wherein the —NH— optionally is substituted with a substituent selected from the group consisting of alkyl, alkenyl, alkynyl, alkoxyalkyl, carbocyclyl, and carbocyclylalkyl, wherein any such substituent is optionally substituted with one or more independently selected halogen.
   In some such embodiments,
X⁹ is selected from the group consisting of —O—, —C(O)—, —S—, —S(O)—, —S(O)₂—, and —NH—, wherein the —NH— optionally is substituted with a substituent selected from the group consisting of alkyl, alkenyl, alkynyl, alkoxyalkyl, carbocyclyl, and carbocyclylalkyl, wherein any such substituent is optionally substituted with one or more independently selected halogen, and the compound has no mirror-symmetry plane.
   In some such embodiments,
at least one of X⁴, X⁵, X⁶ is different from a bond and from —CH₂—, or X⁷ is different from —CH₂—.
   In some such embodiments,
at least one of X⁴, X⁵, X⁶ is different from a bond and from —CH₂—, or X⁷ is different from —CH₂—, and the compound has no mirror-symmetry plane.
   In some such embodiments,
X¹ is selected from the group consisting of phenyl, 5-member heteroaryl, 6-member heteroaryl and C₃-C₆-alkyl wherein:
   the 5-member heteroaryl is optionally substituted by one or more alkyl wherein:
      the alkyl is optionally substituted with one or more independently selected halogen,
   the phenyl and 6-member heteroaryl are optionally substituted at the meta and para positions by one or more substituents selected from the group consisting of alkyl, halogen, alkoxy, arylalkoxy, aryl, cyano and aryloxy wherein:
      the alkyl and alkoxy are optionally substituted with one or more independently selected halogen;
      the arylalkoxy is optionally substituted with one or more haloalkyl; and
   the phenyl is optionally substituted at the ortho positions with one or two independently selected halogen;
X² is selected from the group consisting of a bond, —CH₂—O—, —C(O)—, —N(H)— and —C(S)—;
X⁴ is selected from the group consisting of a bond, —CH₂—, —O—, and —C(O)—, wherein:
   the —CH₂— is optionally substituted with up to two independently selected alkyl;
X⁵ is selected from the group consisting of a bond and —CH₂—;
X⁶ is selected from the group consisting of a bond, —CH₂— and cycloalkyl wherein:
   the —CH₂— is optionally substituted with up to two independently selected alkyl;
X⁷ is selected from the group consisting of —C(O)—, —C(S)—, —NH—C(O)—, —C(O)—NH—, —C(S)—NH—, —S(O)₂— and —C(O)—NH— wherein:
   the —NH—C(O)— and —NH—C(S)— are optionally substituted with alkyl;
X⁹ is selected from the group consisting of —O—, —C(O)—, —S—, —S(O)—, —S(O)₂—, and —NH—, wherein the —NH— optionally is substituted with a substituent selected from the group consisting of alkyl, alkenyl, alkynyl, alkoxyalkyl, carbocyclyl, and carbocyclylalkyl, wherein any such substituent is optionally substituted with one or more independently selected halogen,
Z¹ is selected from the group consisting of N and CH, wherein:
   the CH is optionally substituted with a substituent selected from the group consisting of nitro, halogen, cyano, alkyl, alkoxy, alkylsulfanyl, alkylsulfinyl, alkylsulfonyl, arylsulfonyl, heteroaryl, aminosulfonyl and alkoxycarbonyl wherein:
      the alkyl, alkoxy, alkylsulfanyl, arylsulfonyl, heteroaryl and aminosulfonyl are optionally substituted with one or more substituents independently selected from the group consisting of halogen and alkyl;
Z² is selected from the group consisting of N and CH, wherein:
   the CH is optionally substituted with a substituent selected from the group consisting of alkyl, halogen, cyano, alkoxy, haloalkyl, alkylsulfanyl and haloalkylsulfanyl;
Z³ and Z⁴ are independently selected from the group consisting of N and CH; and
Z⁵ is CH.
   In some such embodiments,
X¹ is selected from the group consisting of phenyl, 5-member heteroaryl, 6-member heteroaryl and C₃-C₆-alkyl wherein:
   the 5-member heteroaryl is optionally substituted by one or more alkyl wherein:
      the alkyl is optionally substituted with one or more independently selected halogen,
   the phenyl and 6-member heteroaryl are optionally substituted at the meta and para positions by one or more substituents selected from the group consisting of alkyl, halogen, alkoxy, arylalkoxy, aryl, cyano and aryloxy wherein:
      the alkyl and alkoxy are optionally substituted with one or more independently selected halogen;
      the arylalkoxy is optionally substituted with one or more haloalkyl; and
   the phenyl is optionally substituted at the ortho positions with one or two independently selected halogen;
X² is selected from the group consisting of a bond, —CH₂—O—, —C(O)—, —N(H)— and —C(S)—;
X⁴ is selected from the group consisting of a bond, —CH₂—, —O—, and —C(O)—, wherein:
   the —CH₂— is optionally substituted with up to two independently selected alkyl;
X⁵ is selected from the group consisting of a bond and —CH₂—;
X⁶ is selected from the group consisting of a bond, —CH₂— and cycloalkyl wherein:
   the —CH₂— is optionally substituted with up to two independently selected alkyl;
X⁷ is selected from the group consisting of —C(O)—, —C(S)—, —NH—C(O)—, —C(O)—NH—, —C(S)—NH—, —S(O)₂— and —C(O)—NH— wherein:
   the —NH—C(O)— and —NH—C(S)— are optionally substituted with alkyl;
X⁹ is selected from the group consisting of —O—, —C(O)—, —S—, —S(O)—, —S(O)₂—, and —NH—, wherein the —NH— optionally is substituted with a substituent selected from the group consisting of alkyl, alkenyl, alkynyl, alkoxyalkyl, carbocyclyl, and carbocyclylalkyl, wherein any such substituent is optionally substituted with one or more independently selected halogen,
Z¹ is selected from the group consisting of N and CH, wherein:
   the CH is optionally substituted with a substituent selected from the group consisting of nitro, halogen, cyano, alkyl, alkoxy, alkylsulfanyl, alkylsulfinyl, alkylsulfonyl, arylsulfonyl, heteroaryl, aminosulfonyl and alkoxycarbonyl wherein:
      the alkyl, alkoxy, alkylsulfanyl, arylsulfonyl, heteroaryl and aminosulfonyl are optionally substituted with one or more substituents independently selected from the group consisting of halogen and alkyl;
Z² is selected from the group consisting of N and CH, wherein:
   the CH is optionally substituted with a substituent selected from the group consisting of alkyl, halogen, cyano, alkoxy, haloalkyl, alkylsulfanyl and haloalkylsulfanyl;
Z³ and Z⁴ are independently selected from the group consisting of N and CH; and
Z⁵ is CH, and the compound has no mirror-symmetry plane.
   In some such embodiments,
X¹ is selected from the group consisting of phenyl, pyridyl and thiadiazoyl, substituted by halogen, (C₁-C₆)alkyl, (C₁-C₆)alkyloxy, (C₁-C₆)haloalkyl, (C₁-C₆)haloalkyloxy, phenyloxy, halophenyloxy, benzyloxy and halobenzyloxy, preferably (C₁-C₆)alkyl, (C₁-C₆)alkyloxy, (C₁-C₆)haloalkyl, (C₁-C₆)haloalkyloxy,
X² is a bond,

X³ is piperazinyl,
X⁴ is —CH₂—,
X⁵ is selected from the group consisting of is —CH₂— and —CH(C₁-C₆)alkyl,
X⁶ is selected from the group consisting of —CH₂— and a bond,
X⁷ is CO or CS,
X⁸ is piperidinyl,
X⁹ is NH or S, preferably NH,
Z¹ is selected from the group consisting of C—NO₂, C—CN, C—S—(C₁-C₆)alkyl and C—S—(C₁-C₆)haloalkyl, preferably C—NO₂ or C—CN,
Z² is C—CF₃ or CH,
Z³ is CH or N,
Z⁴ is CH, and
Z⁵ is CH.
   In some such embodiments,
X¹ is selected from the group consisting of phenyl, pyridyl and thiadiazoyl, substituted by halogen, (C₁-C₆)alkyl, (C₁-C₆)alkyloxy, (C₁-C₆)haloalkyl, (C₁-C₆)haloalkyloxy, phenyloxy, halophenyloxy, benzyloxy and halobenzyloxy, preferably (C₁-C₆)alkyl, (C₁-C₆)alkyloxy, (C₁-C₆)haloalkyl, (C₁-C₆)haloalkyloxy,
X² is a bond,
X³ is piperazinyl,
X⁴ is —CH₂—,
X⁵ is selected from the group consisting of is —CH₂— and —CH(C₁-C₆)alkyl,
X⁶ is selected from the group consisting of —CH₂— and a bond,
X⁷ is CO or CS,
X⁸ is piperidinyl,
X⁹ is NH or S, preferably NH,
Z¹ is selected from the group consisting of C—NO₂, C—CN, C—S—(C₁-C₆)alkyl and C—S—(C₁-C₆)haloalkyl, preferably C—NO₂ or C—CN,
Z² is C—CF₃ or CH,
Z³ is CH or N,
Z⁴ is CH, and
Z⁵ is CH, and the compound has no mirror-symmetry plane.

In some embodiments of this invention, the compound is defined as corresponding in structure to the following formula:

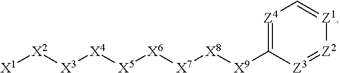

In some such embodiments,
X¹ is selected from the group consisting of phenyl, 5-member heteroaryl, and 6-member heteroaryl, and C₃-C₆-alkyl wherein:
   the 5-member heteroaryl is optionally substituted by one or more alkyl wherein:
      the alkyl is optionally substituted with one or more independently selected halogen,
   the phenyl and 6-member heteroaryl are optionally substituted at the meta and para positions by one or more substituents selected from the group consisting of alkyl, halogen, aryloxy, alkoxy, arylalkoxy and cyano wherein:
      the alkyl is optionally substituted with one or more independently selected halogen;
      the arylalkoxy is optionally substituted with one or more haloalkyl;
   the phenyl is optionally substituted at the ortho position with one or more halogen; and
X² is selected from the group consisting of a bond, —C(O)—, and —CH₂—O—;
X³ is selected from the group consisting of

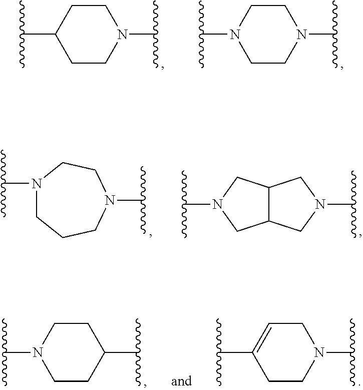

X⁴ is selected from the group consisting of a bond, —CH₂—, —O—, and —C(O)—, wherein:
   the —CH₂— is optionally substituted with up to two substituents independently selected alkyl;
X⁵ is selected from the group consisting of a bond and —CH₂—;
X⁶ is selected from the group consisting of a bond and —CH₂—, wherein:
   the —CH₂— is optionally substituted with up to two substituents independently selected alkyl;
X⁷ is selected from the group consisting of —C(O)—, —C(S)—, —NH—C(O)—, —C(O)—NH—, S(O)₂, and —C(S)—NH— wherein:
   the —NH—C(O)— is optionally substituted with alkyl;
X⁸ is selected from the group consisting of

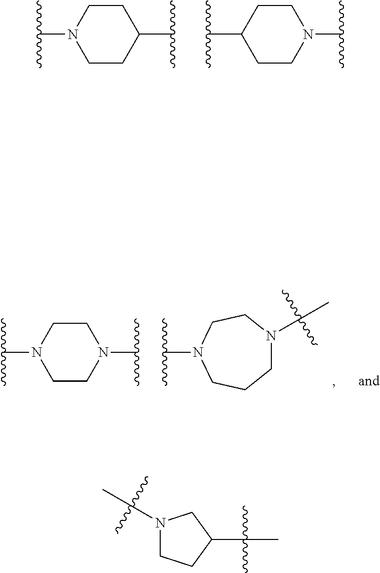

X⁹ is selected from the group consisting of a bond, —NH—, and −O—;
Z¹ is selected from the group consisting of N and CH, wherein:
   the CH is optionally substituted with a substituent selected from the group consisting of nitro, halogen, cyano, alkyl, alkoxy, alkylsulfinyl, alkylsulfanyl, alkylsulfonyl, arylsulfonyl, aminosulfonyl, and 5-membered heteroaryl, wherein:
      the alkyl, alkoxy, alkylsulfanyl, arylsulfonyl, aminosulfonyl, and 5-membered heteroaryl are optionally substituted with one or more substituents independently selected from the group consisting of halogen and alkyl;
Z² is selected from the group consisting of N and CH, wherein:
   the CH is optionally substituted with a substituent selected from the group consisting of alkyl, halogen, cyano, alkoxy, haloalkyl and haloalkylsulfanyl; and
Z³ and Z⁴ are independently selected from the group consisting of N and CH.

In some embodiments of this invention, the compound is defined as corresponding in structure to the following formula:

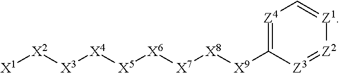

In some such embodiments,
X¹ is selected from the group consisting of phenyl, 5-member heteroaryl, and 6-member heteroaryl, and C₃-C₆-alkyl wherein:
   the 5-member heteroaryl is optionally substituted by one or more alkyl wherein:
      the alkyl is optionally substituted with one or more independently selected halogen,
   the phenyl and 6-member heteroaryl are optionally substituted at the meta and para positions by one or more substituents selected from the group consisting of alkyl, halogen, aryloxy, alkoxy, arylalkoxy and cyano wherein:
      the alkyl is optionally substituted with one or more independently selected halogen;
      the arylalkoxy is optionally substituted with one or more haloalkyl;
   the phenyl is optionally substituted at the ortho position with one or more halogen; and
X² is selected from the group consisting of a bond, —C(O)—, and —CH₂—O—;
X³ is selected from the group consisting of

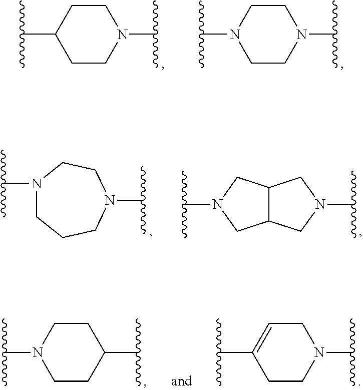

X⁴ is selected from the group consisting of a bond, —CH₂—, —O—, and —C(O)—, wherein:
   the —CH₂— is optionally substituted with up to two substituents independently selected alkyl;
X⁵ is selected from the group consisting of a bond and —CH₂—;
X⁶ is selected from the group consisting of a bond and —CH₂—, wherein:
   the —CH₂— is optionally substituted with up to two substituents independently selected alkyl;
X⁷ is selected from the group consisting of —C(O)—, —C(S)—, —NH—C(O)—, —C(O)—NH—, S(O)₂, and —C(S)—NH— wherein:
   the —NH—C(O)— is optionally substituted with alkyl;
X⁸ is selected from the group consisting of

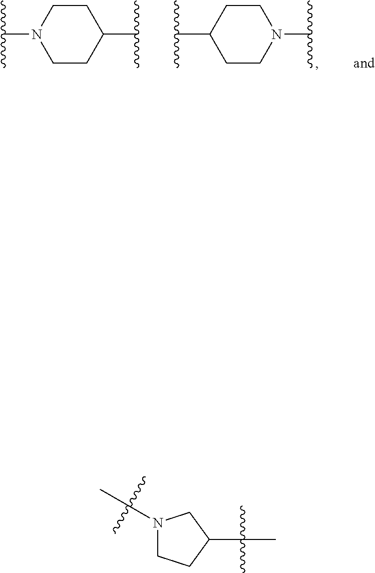

X⁹ is selected from the group consisting of a bond, —NH—, and −O—;
Z¹ is selected from the group consisting of N and CH, wherein:
   the CH is optionally substituted with a substituent selected from the group consisting of nitro, halogen, cyano, alkyl, alkoxy, alkylsulfinyl, alkylsulfanyl, alkylsulfonyl, arylsulfonyl, aminosulfonyl, and 5-membered heteroaryl, wherein:
      the alkyl, alkoxy, alkylsulfanyl, arylsulfonyl, aminosulfonyl, and 5-membered heteroaryl are optionally substituted with one or more substituents independently selected from the group consisting of halogen and alkyl;
Z² is selected from the group consisting of N and CH, wherein:
   the CH is optionally substituted with a substituent selected from the group consisting of alkyl, halogen, cyano, alkoxy, haloalkyl and haloalkylsulfanyl; and
Z³ and Z⁴ are independently selected from the group consisting of N and CH.

In some embodiments, the compound or salt thereof corresponds to a structure selected from the group consisting of:

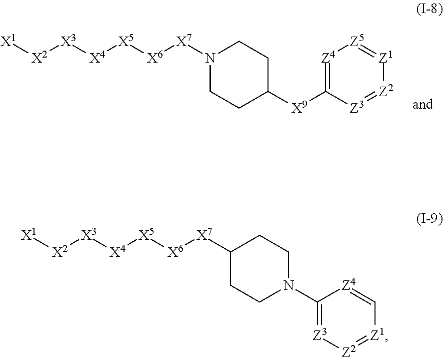

X¹ is selected from the group consisting of phenyl, 5-member heteroaryl, and 6-member heteroaryl, wherein:
   the 5-member heteroaryl is substituted with trifluoromethyl;
   the phenyl and 6-member heteroaryl are optionally substituted at the meta and para positions by one or more substituents selected from the group consisting of alkyl, trifluoromethyl, halogen, phenoxy, alkoxy, and trifluoromethylphenylalkoxy wherein:
X² is selected from the group consisting of a bond and —CH₂—O—;
X³ is a linker selected from the group consisting of:

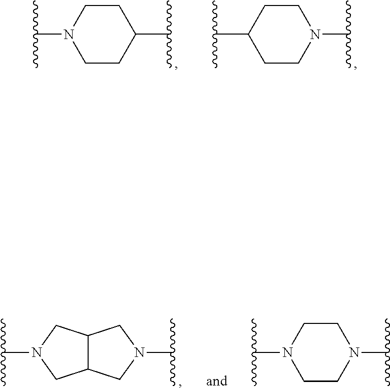

X⁵ is selected from the group consisting of a bond and —CH₂—;
X⁶ is selected from the group consisting of a bond and —CH₂—, wherein:
   the —CH₂— is optionally substituted with up to two substituents independently selected alkyl;
X⁷ is selected from the group consisting of —C(O)—, —C(S)—, —NH—C(O)—, —C(O)—NH—, and —C(S)—NH— wherein:
   the —NH—C(O)— is optionally substituted with alkyl;
X⁹ is selected from the group consisting of a bond, —NH—, and —O;
Z¹ is selected from the group consisting of N and CH, wherein:
   the CH is optionally substituted with a substituent selected from the group consisting of nitro, halogen, cyano, trifluoromethyl, trifluoromethoxy, alkylsulfanyl, trifluoromethylsulfanyl, alkylsulfonyl, trifluormethylsulfonyl, phenylsulfonyl and 5-membered-heteroaryl, wherein:
      the 5-membered-heteroaryl is optionally substituted with C₁-C₃-alkyl;
Z² is selected from the group consisting of N and CH, wherein:
   the CH is optionally substituted with a substituent selected from the group consisting of alkyl, halogen, cyano, alkoxy, trifluoromethyl and trifluoromethylsulfanyl; and
Z³ and Z⁴ are independently selected from the group consisting of N and CH.
   Compounds encompassed by these embodiments include, for example:

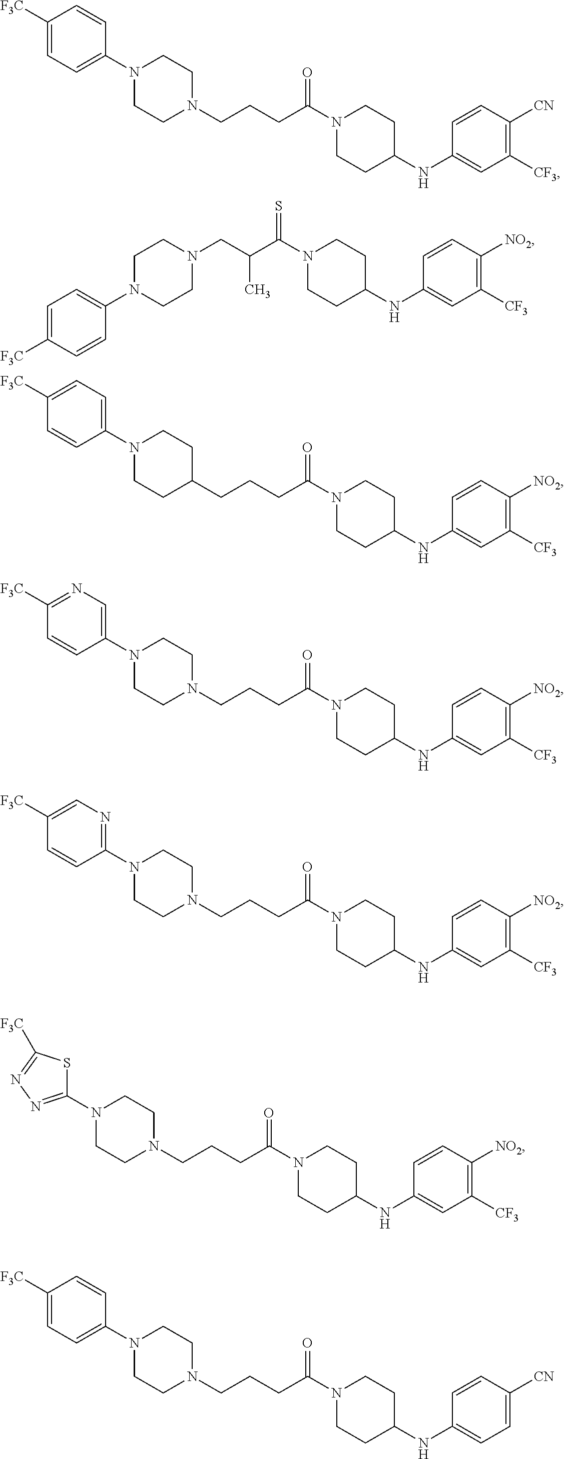

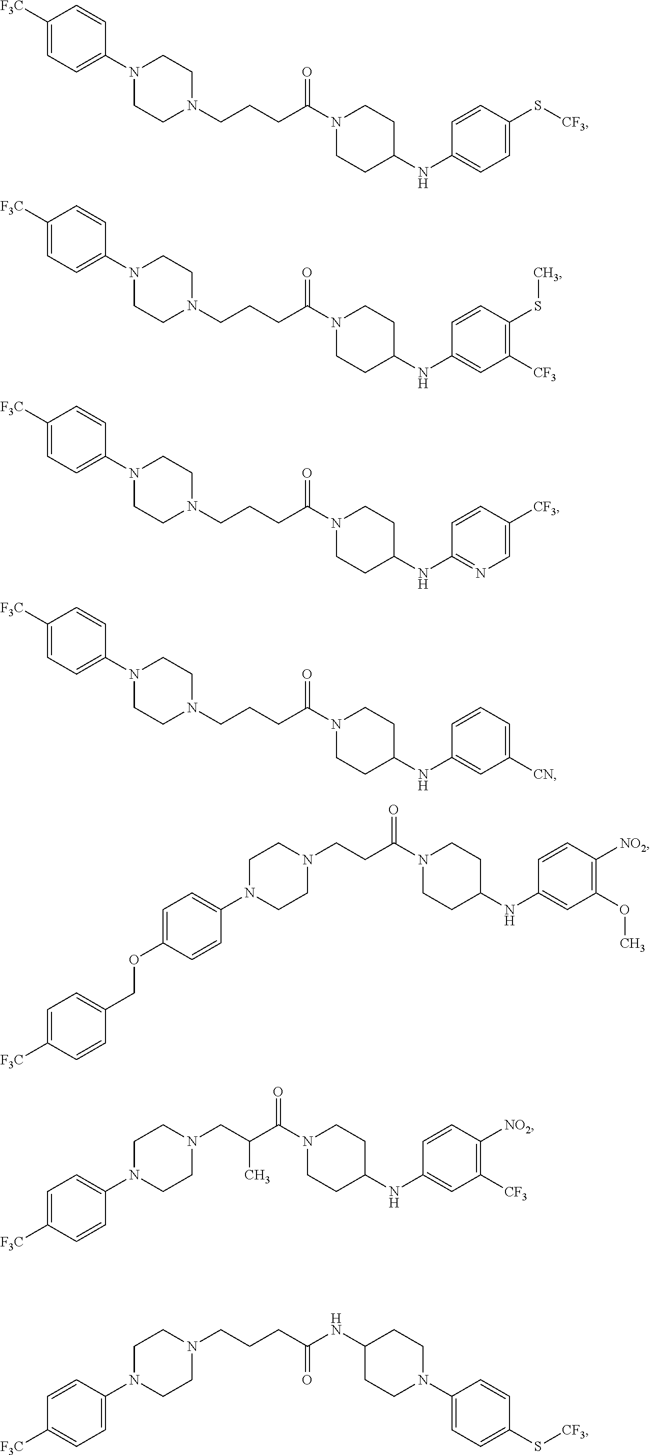

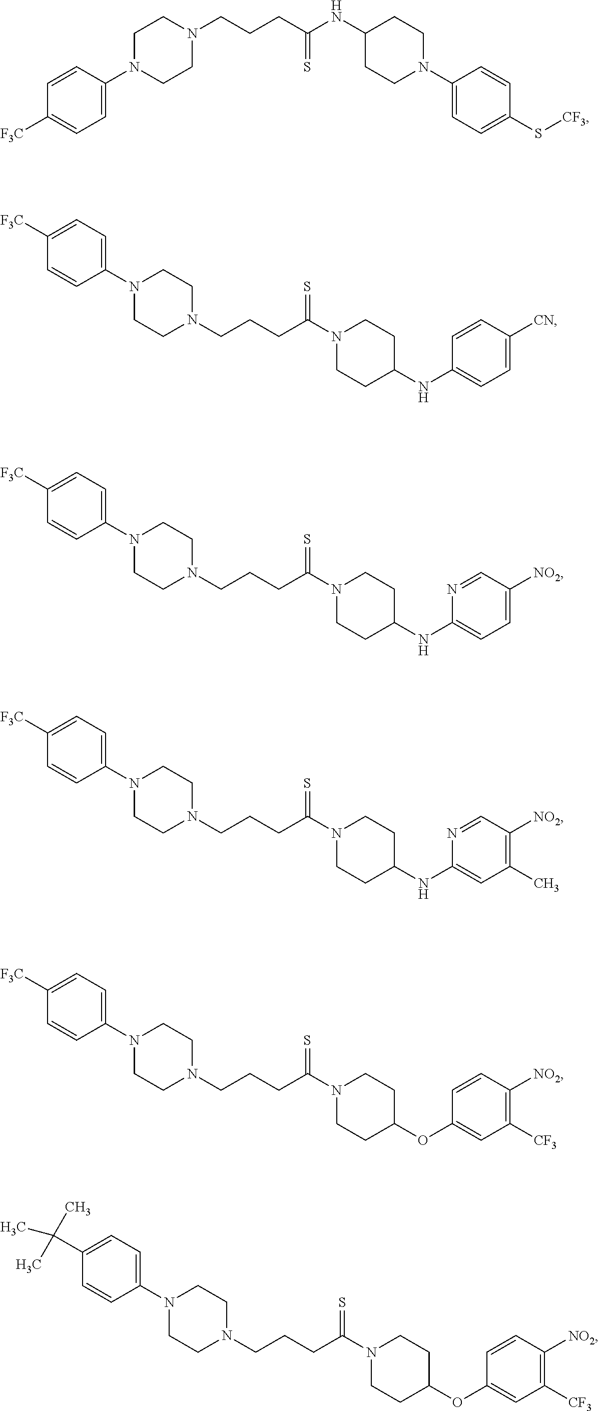 [SMILES: FC(F)(F)Sc1ccc(N2CCC(NC(=S)CCCN3CCN(c4ccc(C(F)(F)F)cc4)CC3)CC2)cc1 ; N#Cc1ccc(NC2CCN(C(=S)CCCN3CCN(c4ccc(C(F)(F)F)cc4)CC3)CC2)cc1 ; O=[N+]([O-])c1ccc(NC2CCN(C(=S)CCCN3CCN(c4ccc(C(F)(F)F)cc4)CC3)CC2)nc1 ; Cc1cc(NC2CCN(C(=S)CCCN3CCN(c4ccc(C(F)(F)F)cc4)CC3)CC2)ncc1[N+](=O)[O-] ; O=[N+]([O-])c1ccc(OC2CCN(C(=S)CCCN3CCN(c4ccc(C(F)(F)F)cc4)CC3)CC2)cc1C(F)(F)F ; CC(C)(C)c1ccc(N2CCN(CCCC(=S)N3CCC(Oc4ccc([N+](=O)[O-])c(C(F)(F)F)c4)CC3)CC2)cc1]

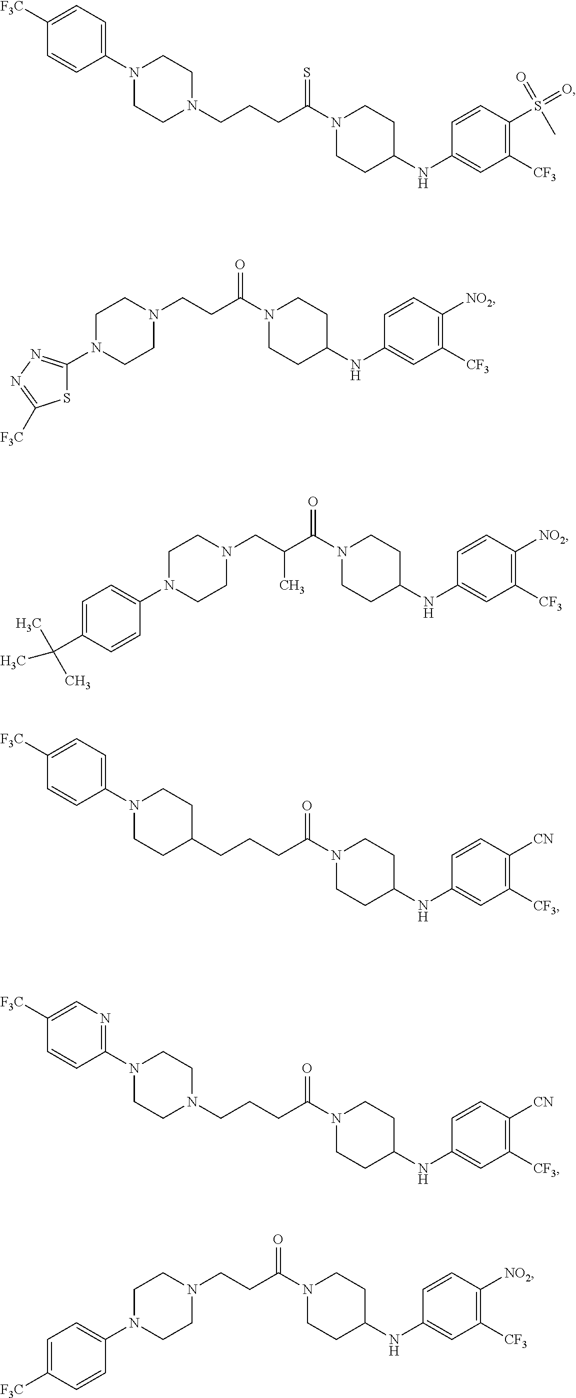 [SMILES: CS(=O)(=O)c1ccc(NC2CCN(C(=S)CCCN3CCN(c4ccc(C(F)(F)F)cc4)CC3)CC2)cc1C(F)(F)F ; O=C(CCN1CCN(c2nnc(C(F)(F)F)s2)CC1)N1CCC(Nc2ccc([N+](=O)[O-])c(C(F)(F)F)c2)CC1 ; CC(CN1CCN(c2ccc(C(C)(C)C)cc2)CC1)C(=O)N1CCC(Nc2ccc([N+](=O)[O-])c(C(F)(F)F)c2)CC1 ; N#Cc1ccc(NC2CCN(C(=O)CCCC3CCN(c4ccc(C(F)(F)F)cc4)CC3)CC2)cc1C(F)(F)F ; N#Cc1ccc(NC2CCN(C(=O)CCCN3CCN(c4ccc(C(F)(F)F)cn4)CC3)CC2)cc1C(F)(F)F ; O=C(CCN1CCN(c2ccc(C(F)(F)F)cc2)CC1)N1CCC(Nc2ccc([N+](=O)[O-])c(C(F)(F)F)c2)CC1]

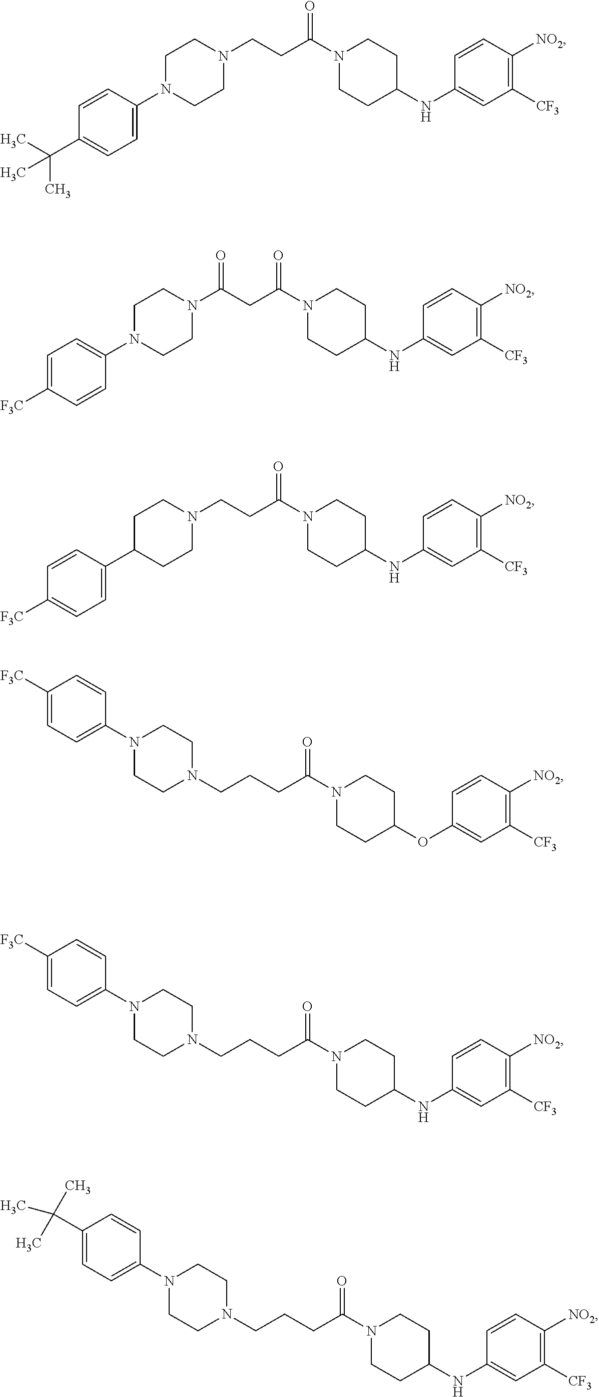 [SMILES: CC(C)(C)c1ccc(N2CCN(CCC(=O)N3CCC(Nc4ccc([N+](=O)[O-])c(C(F)(F)F)c4)CC3)CC2)cc1 ; O=C(CC(=O)N1CCN(c2ccc(C(F)(F)F)cc2)CC1)N1CCC(Nc2ccc([N+](=O)[O-])c(C(F)(F)F)c2)CC1 ; O=C(CCN1CCC(c2ccc(C(F)(F)F)cc2)CC1)N1CCC(Nc2ccc([N+](=O)[O-])c(C(F)(F)F)c2)CC1 ; O=C(CCCN1CCN(c2ccc(C(F)(F)F)cc2)CC1)N1CCC(Oc2ccc([N+](=O)[O-])c(C(F)(F)F)c2)CC1 ; O=C(CCCN1CCN(c2ccc(C(F)(F)F)cc2)CC1)N1CCC(Nc2ccc([N+](=O)[O-])c(C(F)(F)F)c2)CC1 ; CC(C)(C)c1ccc(N2CCN(CCCC(=O)N3CCC(Nc4ccc([N+](=O)[O-])c(C(F)(F)F)c4)CC3)CC2)cc1]

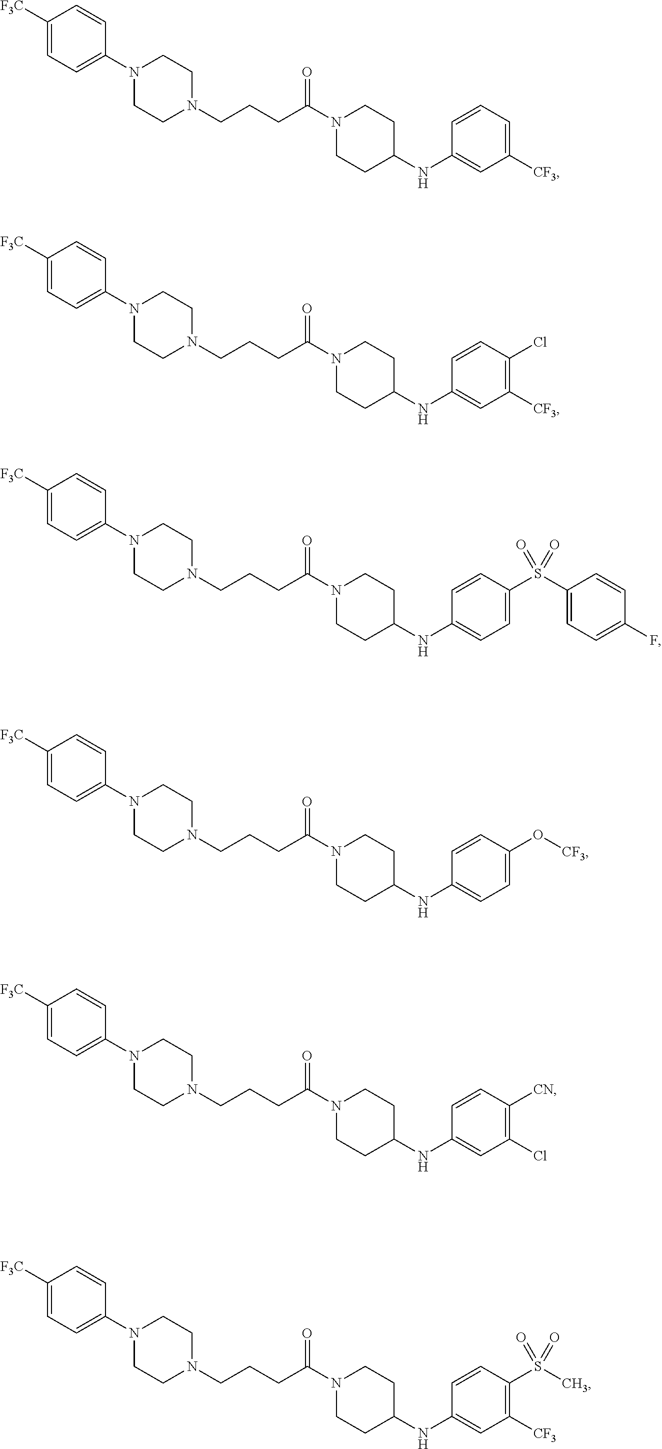 [SMILES: O=C(CCCN1CCN(c2ccc(C(F)(F)F)cc2)CC1)N1CCC(Nc2cccc(C(F)(F)F)c2)CC1 ; O=C(CCCN1CCN(c2ccc(C(F)(F)F)cc2)CC1)N1CCC(Nc2ccc(Cl)c(C(F)(F)F)c2)CC1 ; O=C(CCCN1CCN(c2ccc(C(F)(F)F)cc2)CC1)N1CCC(Nc2ccc(S(=O)(=O)c3ccc(F)cc3)cc2)CC1 ; O=C(CCCN1CCN(c2ccc(C(F)(F)F)cc2)CC1)N1CCC(Nc2ccc(OC(F)(F)F)cc2)CC1 ; N#Cc1ccc(NC2CCN(C(=O)CCCN3CCN(c4ccc(C(F)(F)F)cc4)CC3)CC2)cc1Cl ; CS(=O)(=O)c1ccc(NC2CCN(C(=O)CCCN3CCN(c4ccc(C(F)(F)F)cc4)CC3)CC2)cc1C(F)(F)F]

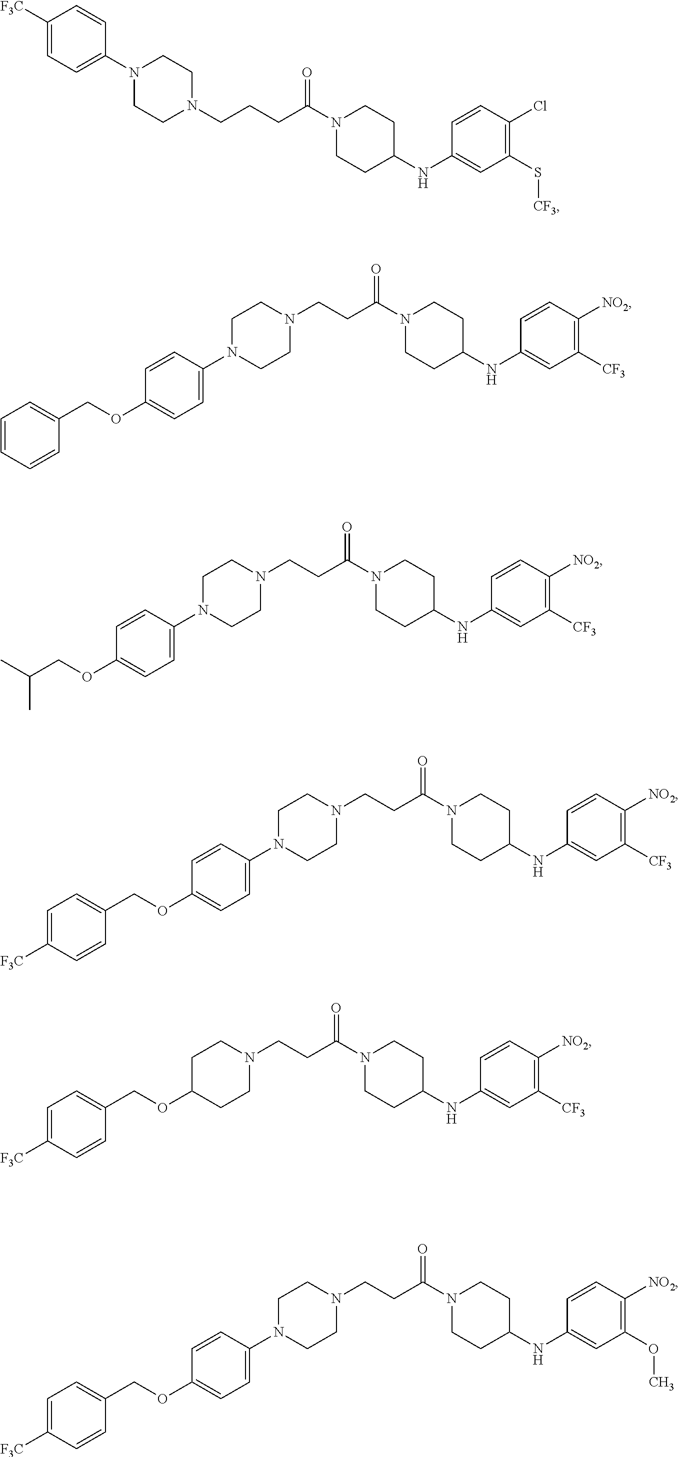 [SMILES: O=C(CCCN1CCN(c2ccc(C(F)(F)F)cc2)CC1)N1CCC(Nc2ccc(Cl)c(SC(F)(F)F)c2)CC1 ; O=C(CCN1CCN(c2ccc(OCc3ccccc3)cc2)CC1)N1CCC(Nc2ccc([N+](=O)[O-])c(C(F)(F)F)c2)CC1 ; CC(C)COc1ccc(N2CCN(CCC(=O)N3CCC(Nc4ccc([N+](=O)[O-])c(C(F)(F)F)c4)CC3)CC2)cc1 ; O=C(CCN1CCN(c2ccc(OCc3ccc(C(F)(F)F)cc3)cc2)CC1)N1CCC(Nc2ccc([N+](=O)[O-])c(C(F)(F)F)c2)CC1 ; O=C(CCN1CCC(OCc2ccc(C(F)(F)F)cc2)CC1)N1CCC(Nc2ccc([N+](=O)[O-])c(C(F)(F)F)c2)CC1 ; COc1cc(NC2CCN(C(=O)CCN3CCN(c4ccc(OCc5ccc(C(F)(F)F)cc5)cc4)CC3)CC2)ccc1[N+](=O)[O-]]

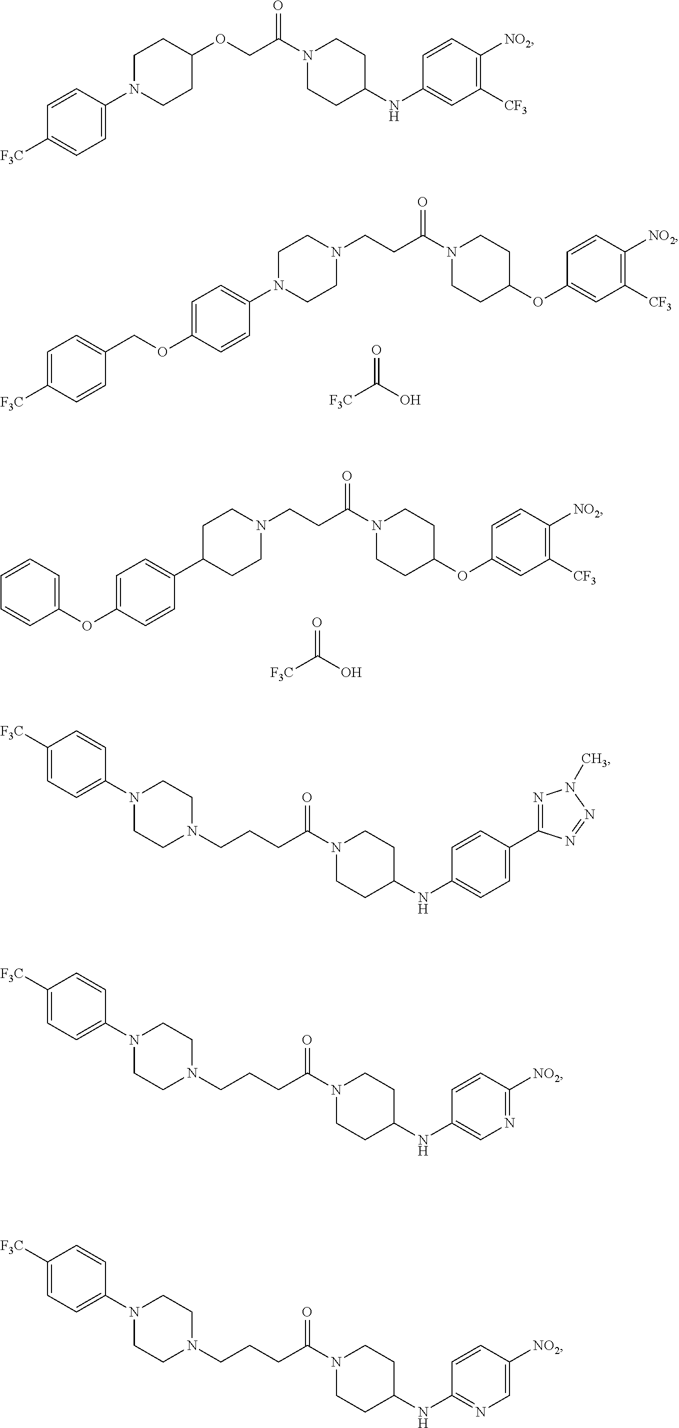

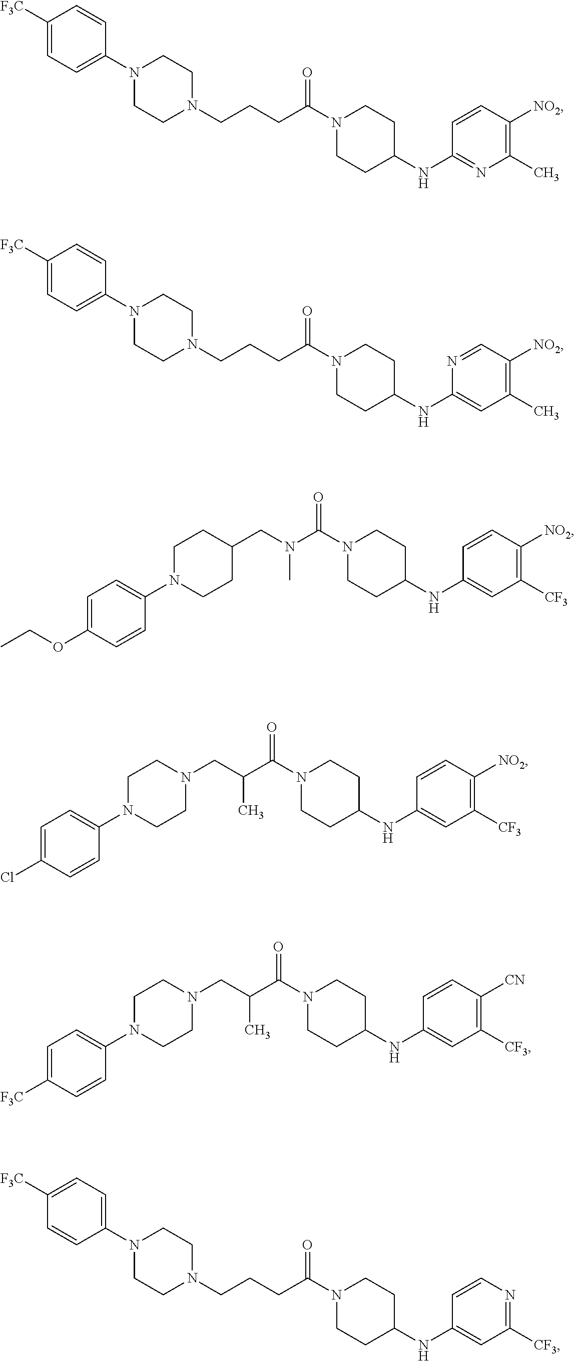 [SMILES: Cc1nc(NC2CCN(C(=O)CCCN3CCN(c4ccc(C(F)(F)F)cc4)CC3)CC2)ccc1[N+](=O)[O-] ; Cc1cc(NC2CCN(C(=O)CCCN3CCN(c4ccc(C(F)(F)F)cc4)CC3)CC2)ncc1[N+](=O)[O-] ; CCOc1ccc(N2CCC(CN(C)C(=O)N3CCC(Nc4ccc([N+](=O)[O-])c(C(F)(F)F)c4)CC3)CC2)cc1 ; CC(CN1CCN(c2ccc(Cl)cc2)CC1)C(=O)N1CCC(Nc2ccc([N+](=O)[O-])c(C(F)(F)F)c2)CC1 ; CC(CN1CCN(c2ccc(C(F)(F)F)cc2)CC1)C(=O)N1CCC(Nc2ccc(C#N)c(C(F)(F)F)c2)CC1 ; O=C(CCCN1CCN(c2ccc(C(F)(F)F)cc2)CC1)N1CCC(Nc2ccnc(C(F)(F)F)c2)CC1]

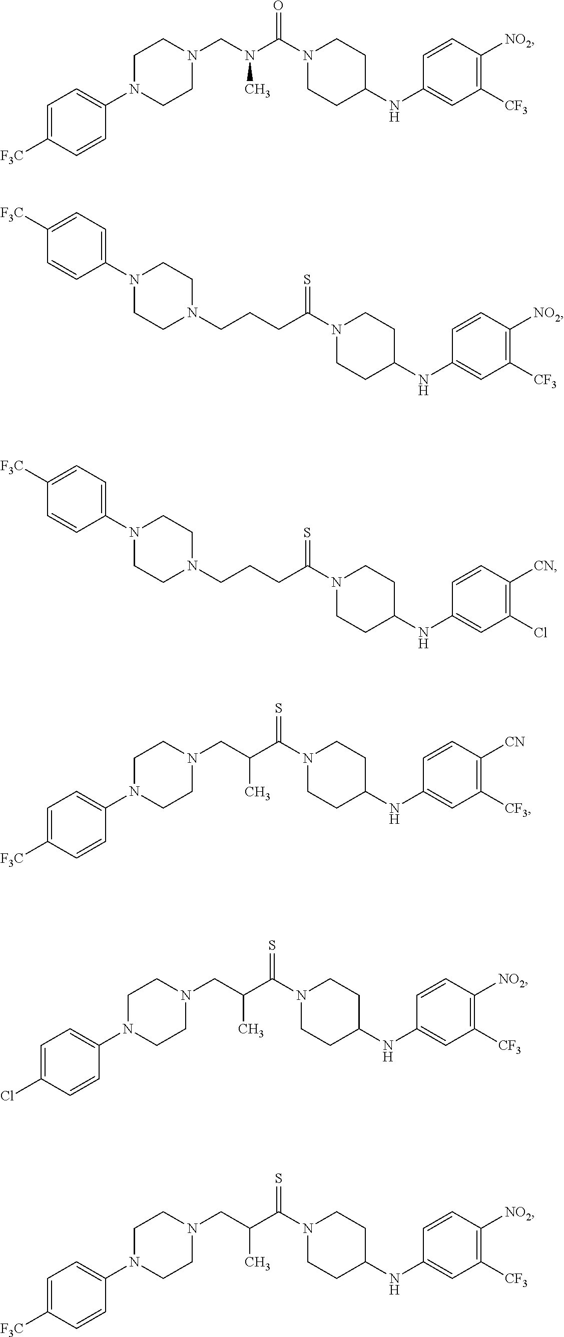

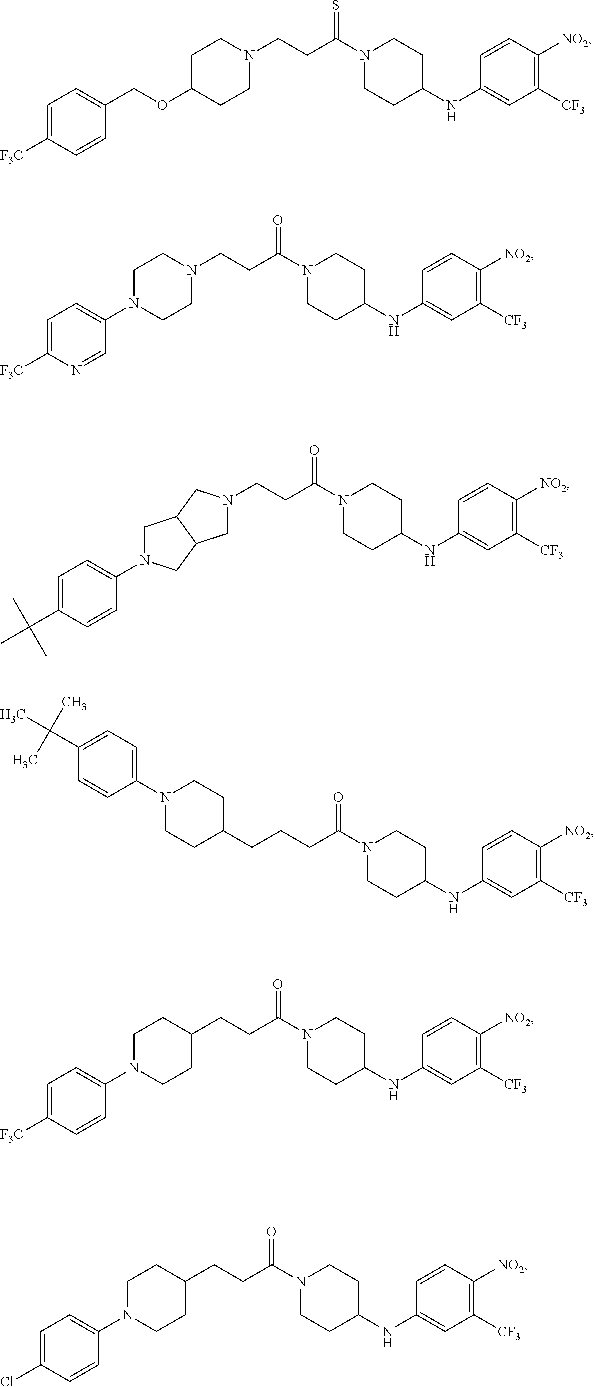 [SMILES: O=[N+]([O-])c1ccc(NC2CCN(C(=S)CCN3CCC(OCc4ccc(C(F)(F)F)cc4)CC3)CC2)cc1C(F)(F)F ; O=C(CCN1CCN(c2ccc(C(F)(F)F)nc2)CC1)N1CCC(Nc2ccc([N+](=O)[O-])c(C(F)(F)F)c2)CC1 ; CC(C)(C)c1ccc(N2CC3CN(CCC(=O)N4CCC(Nc5ccc([N+](=O)[O-])c(C(F)(F)F)c5)CC4)CC3C2)cc1 ; CC(C)(C)c1ccc(N2CCC(CCCC(=O)N3CCC(Nc4ccc([N+](=O)[O-])c(C(F)(F)F)c4)CC3)CC2)cc1 ; O=C(CCC1CCN(c2ccc(C(F)(F)F)cc2)CC1)N1CCC(Nc2ccc([N+](=O)[O-])c(C(F)(F)F)c2)CC1 ; O=C(CCC1CCN(c2ccc(Cl)cc2)CC1)N1CCC(Nc2ccc([N+](=O)[O-])c(C(F)(F)F)c2)CC1]

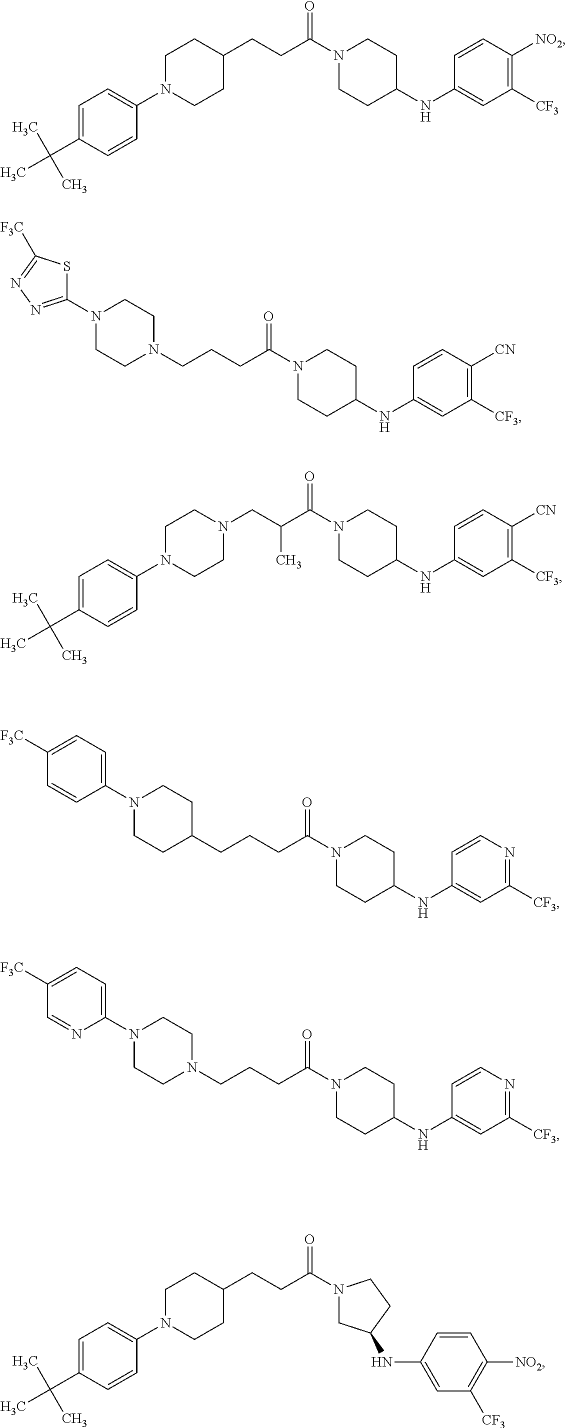 [SMILES: CC(C)(C)c1ccc(N2CCC(CCC(=O)N3CCC(Nc4ccc([N+](=O)[O-])c(C(F)(F)F)c4)CC3)CC2)cc1 ; N#Cc1ccc(NC2CCN(C(=O)CCCN3CCN(c4nnc(C(F)(F)F)s4)CC3)CC2)cc1C(F)(F)F ; CC(CN1CCN(c2ccc(C(C)(C)C)cc2)CC1)C(=O)N1CCC(Nc2ccc(C#N)c(C(F)(F)F)c2)CC1 ; O=C(CCCC1CCN(c2ccc(C(F)(F)F)cc2)CC1)N1CCC(Nc2ccnc(C(F)(F)F)c2)CC1 ; O=C(CCCN1CCN(c2ccc(C(F)(F)F)cn2)CC1)N1CCC(Nc2ccnc(C(F)(F)F)c2)CC1 ; CC(C)(C)c1ccc(N2CCC(CCC(=O)N3CC[C@@H](Nc4ccc([N+](=O)[O-])c(C(F)(F)F)c4)C3)CC2)cc1]

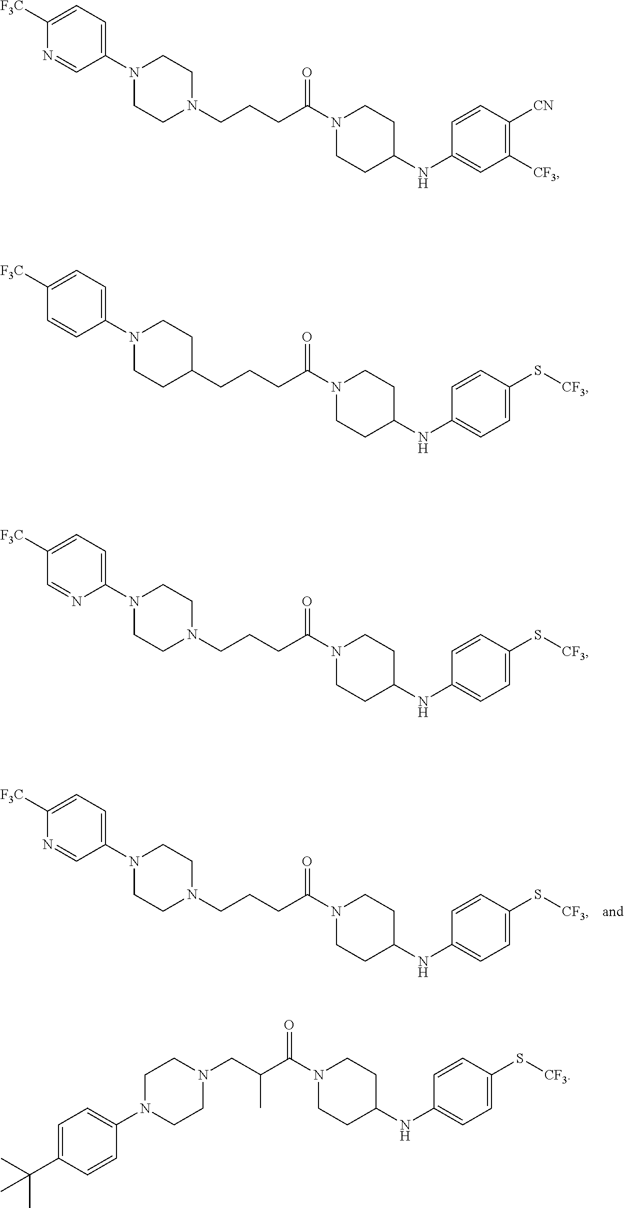 [SMILES: N#Cc1ccc(NC2CCN(C(=O)CCCN3CCN(c4ccc(C(F)(F)F)nc4)CC3)CC2)cc1C(F)(F)F ; O=C(CCCC1CCN(c2ccc(C(F)(F)F)cc2)CC1)N1CCC(Nc2ccc(SC(F)(F)F)cc2)CC1 ; O=C(CCCN1CCN(c2ccc(C(F)(F)F)cn2)CC1)N1CCC(Nc2ccc(SC(F)(F)F)cc2)CC1 ; O=C(CCCN1CCN(c2ccc(C(F)(F)F)nc2)CC1)N1CCC(Nc2ccc(SC(F)(F)F)cc2)CC1 ; "and" ; CC(CN1CCN(c2ccc(C(C)(C)C)cc2)CC1)C(=O)N1CCC(Nc2ccc(SC(F)(F)F)cc2)CC1]

In some embodiments, the compound or salt thereof corresponds to a structure selected from the group consisting of:

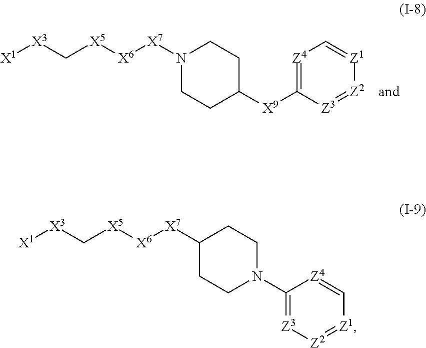

X¹ is selected from the group consisting of phenyl, 5-member heteroaryl, and 6-member heteroaryl, wherein:
the 5-member heteroaryl is substituted with trifluoromethyl;
the phenyl and 6-member heteroaryl are optionally substituted at the para position by a substituent selected from the group consisting of C₁-C₄-alkyl, trifluoromethyl, and trifluorophenyl-C₁-C₃-alkoxy;

X³ is a linker selected from the group consisting of:

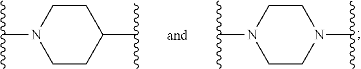 [SMILES: *C1CCN(*)CC1 ; "and" ; *N1CCN(*)CC1 ; ";"]

X⁵ is selected from the group consisting of a bond and —CH₂—;

X⁶ is —CH₂—, optionally substituted with C₁-C₃-alkyl;

X⁷ is selected from the group consisting of —C(O)—, —C(S), —C(O)—NH—, and —C(S)—NH—;

X⁹ is selected from the group consisting of —NH— and —O;

Z¹ is CH, wherein:
the CH is optionally substituted with a substituent selected from the group consisting of nitro, cyano, alkyl, alkylsulfanyl and alkylsulfonyl, wherein:
   the alkyl and alkylsulfanyl are optionally substituted with one or more halogen;

Z² is CH, wherein:
the CH is optionally substituted with a substituent selected from the group consisting of trifluoromethyl and C₁-C₃-alkoxy; and

Z³ and Z⁴ are independently selected from the group consisting of N and CH.

Compounds encompassed by these embodiments include, for example:

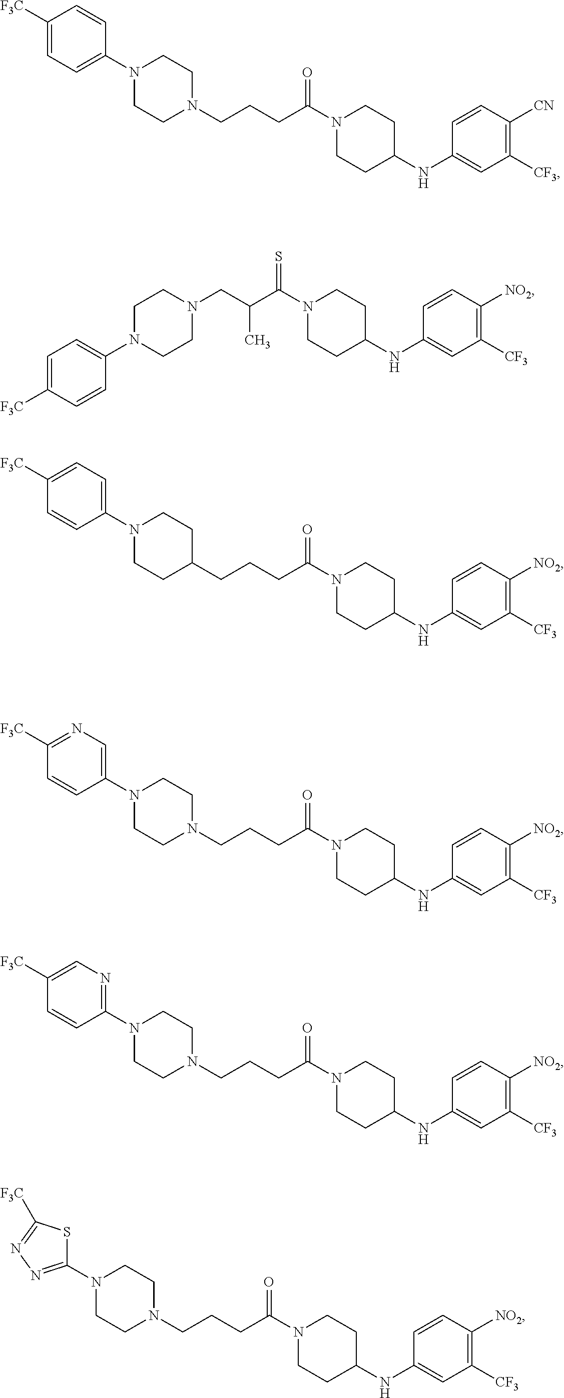 [SMILES: N#Cc1ccc(NC2CCN(C(=O)CCCN3CCN(c4ccc(C(F)(F)F)cc4)CC3)CC2)cc1C(F)(F)F ; CC(CN1CCN(c2ccc(C(F)(F)F)cc2)CC1)C(=S)N1CCC(Nc2ccc([N+](=O)[O-])c(C(F)(F)F)c2)CC1 ; O=C(CCCC1CCN(c2ccc(C(F)(F)F)cc2)CC1)N1CCC(Nc2ccc([N+](=O)[O-])c(C(F)(F)F)c2)CC1 ; O=C(CCCN1CCN(c2ccc(C(F)(F)F)nc2)CC1)N1CCC(Nc2ccc([N+](=O)[O-])c(C(F)(F)F)c2)CC1 ; O=C(CCCN1CCN(c2ccc(C(F)(F)F)cn2)CC1)N1CCC(Nc2ccc([N+](=O)[O-])c(C(F)(F)F)c2)CC1 ; O=C(CCCN1CCN(c2nnc(C(F)(F)F)s2)CC1)N1CCC(Nc2ccc([N+](=O)[O-])c(C(F)(F)F)c2)CC1]

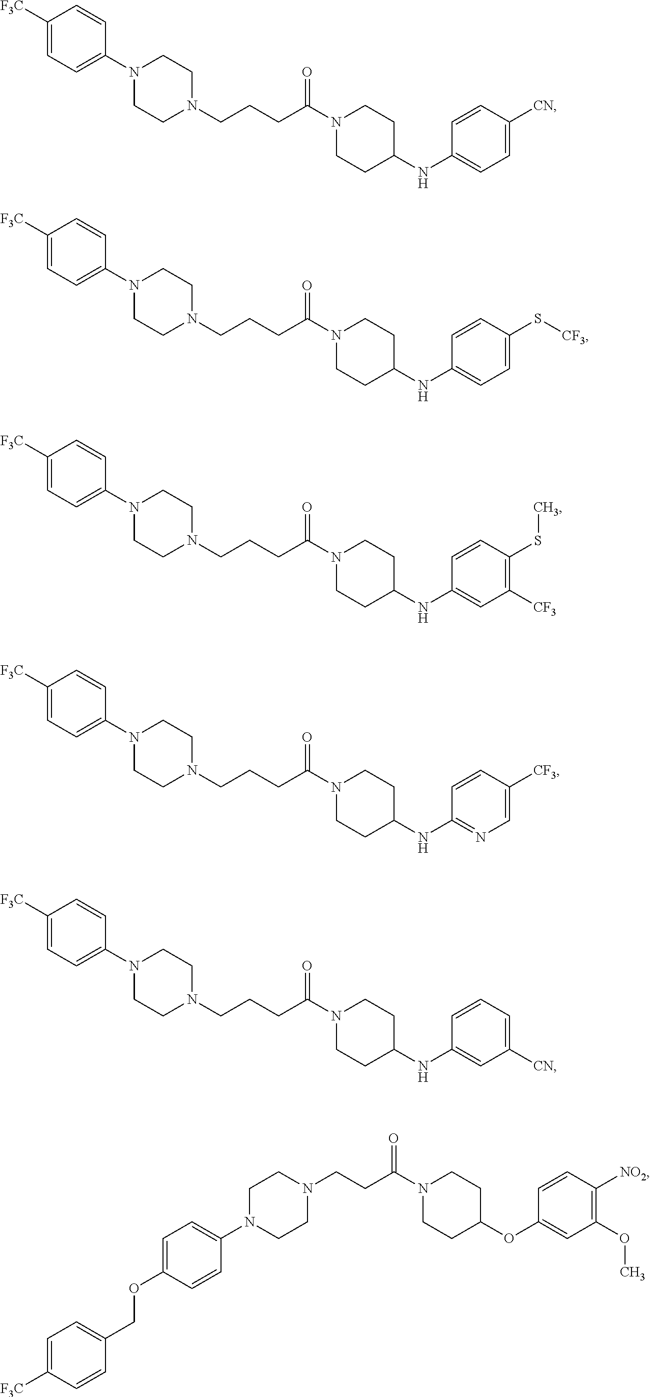 [SMILES: N#Cc1ccc(NC2CCN(C(=O)CCCN3CCN(c4ccc(C(F)(F)F)cc4)CC3)CC2)cc1 ; O=C(CCCN1CCN(c2ccc(C(F)(F)F)cc2)CC1)N1CCC(Nc2ccc(SC(F)(F)F)cc2)CC1 ; CSc1ccc(NC2CCN(C(=O)CCCN3CCN(c4ccc(C(F)(F)F)cc4)CC3)CC2)cc1C(F)(F)F ; O=C(CCCN1CCN(c2ccc(C(F)(F)F)cc2)CC1)N1CCC(Nc2ccc(C(F)(F)F)cn2)CC1 ; N#Cc1cccc(NC2CCN(C(=O)CCCN3CCN(c4ccc(C(F)(F)F)cc4)CC3)CC2)c1 ; COc1cc(OC2CCN(C(=O)CCN3CCN(c4ccc(OCc5ccc(C(F)(F)F)cc5)cc4)CC3)CC2)ccc1[N+](=O)[O-]]

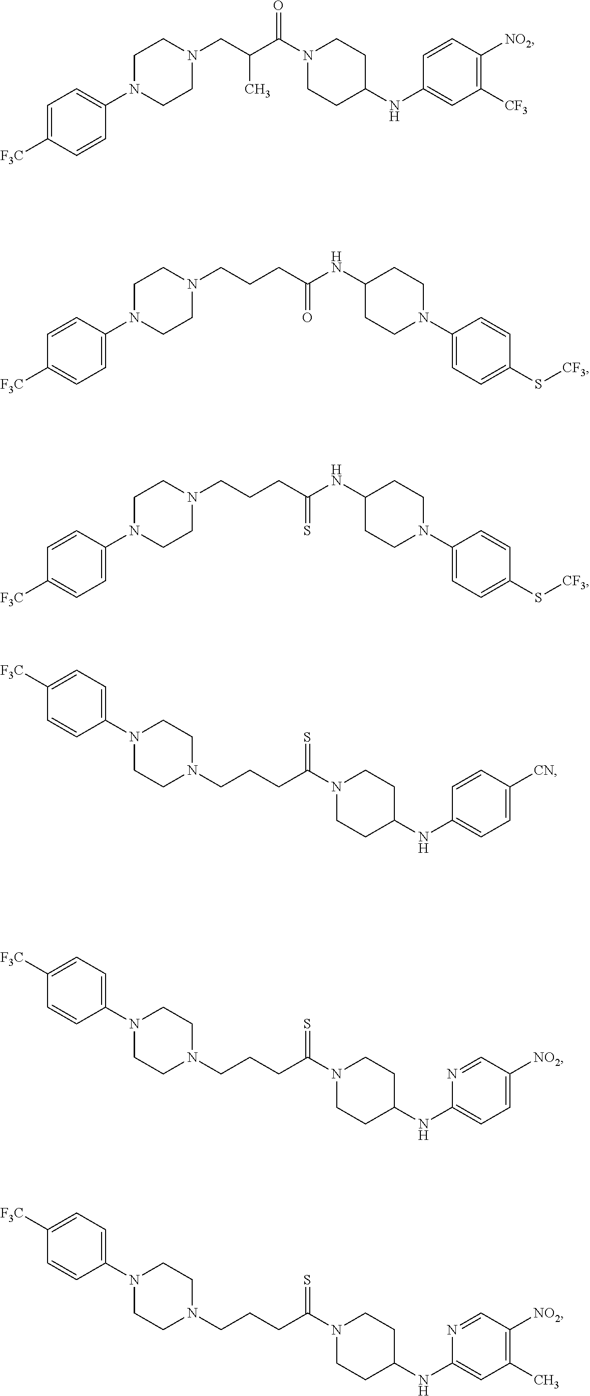 [SMILES: CC(CN1CCN(c2ccc(C(F)(F)F)cc2)CC1)C(=O)N1CCC(Nc2ccc([N+](=O)[O-])c(C(F)(F)F)c2)CC1 ; O=C(CCCN1CCN(c2ccc(C(F)(F)F)cc2)CC1)NC1CCN(c2ccc(SC(F)(F)F)cc2)CC1 ; FC(F)(F)Sc1ccc(N2CCC(NC(=S)CCCN3CCN(c4ccc(C(F)(F)F)cc4)CC3)CC2)cc1 ; N#Cc1ccc(NC2CCN(C(=S)CCCN3CCN(c4ccc(C(F)(F)F)cc4)CC3)CC2)cc1 ; O=[N+]([O-])c1ccc(NC2CCN(C(=S)CCCN3CCN(c4ccc(C(F)(F)F)cc4)CC3)CC2)nc1 ; Cc1cc(NC2CCN(C(=S)CCCN3CCN(c4ccc(C(F)(F)F)cc4)CC3)CC2)ncc1[N+](=O)[O-]]

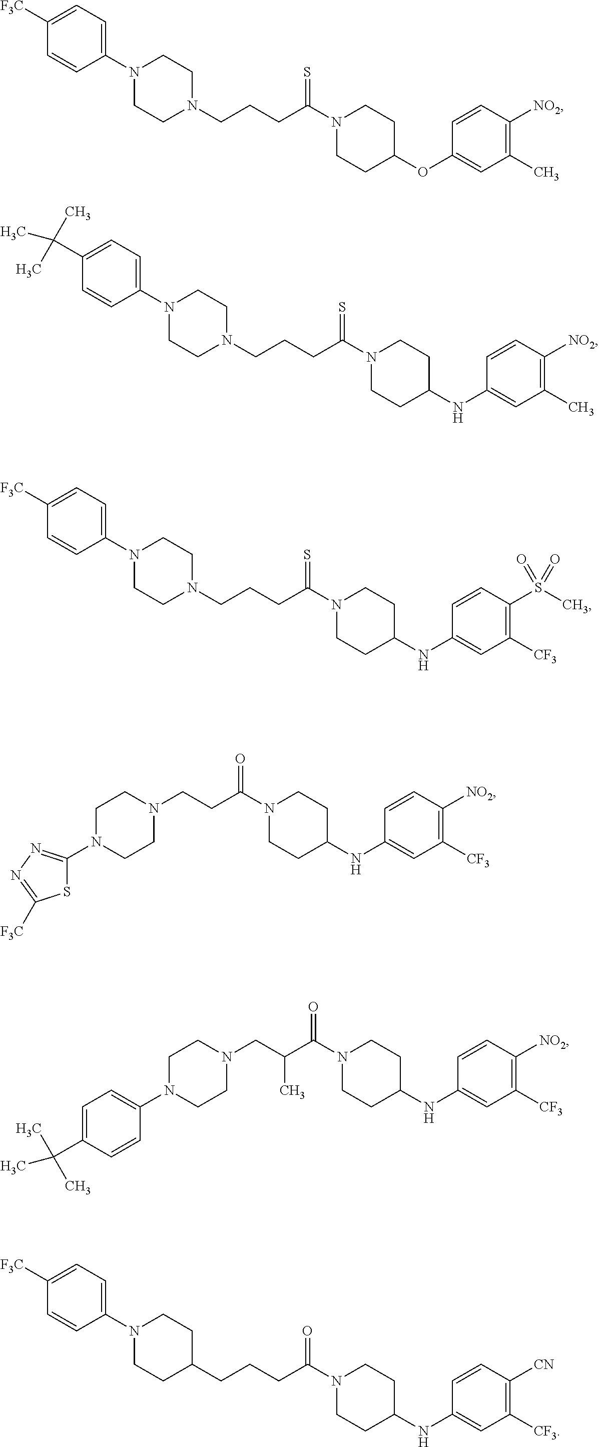 [SMILES: Cc1cc(OC2CCN(C(=S)CCCN3CCN(c4ccc(C(F)(F)F)cc4)CC3)CC2)ccc1[N+](=O)[O-] ; Cc1cc(NC2CCN(C(=S)CCCN3CCN(c4ccc(C(C)(C)C)cc4)CC3)CC2)ccc1[N+](=O)[O-] ; CS(=O)(=O)c1ccc(NC2CCN(C(=S)CCCN3CCN(c4ccc(C(F)(F)F)cc4)CC3)CC2)cc1C(F)(F)F ; O=C(CCN1CCN(c2nnc(C(F)(F)F)s2)CC1)N1CCC(Nc2ccc([N+](=O)[O-])c(C(F)(F)F)c2)CC1 ; CC(CN1CCN(c2ccc(C(C)(C)C)cc2)CC1)C(=O)N1CCC(Nc2ccc([N+](=O)[O-])c(C(F)(F)F)c2)CC1 ; N#Cc1ccc(NC2CCN(C(=O)CCCC3CCN(c4ccc(C(F)(F)F)cc4)CC3)CC2)cc1C(F)(F)F]

In some embodiments, the compound or salt thereof corresponds in structure to:

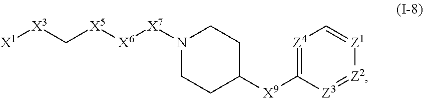

wherein

X⁹ is selected from the group consisting of —NH— and −O—.

In some embodiments, the compound or salt thereof corresponds in structure to:

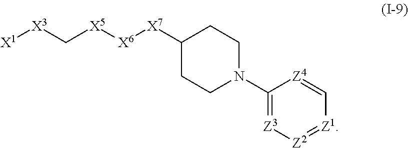

In some embodiments, the compound or salt thereof corresponds in structure to:

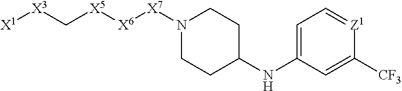

X¹ is selected from the group consisting of phenyl, 5-member heteroaryl, and 6-member heteroaryl, wherein:
the 5-member heteroaryl is substituted with trifluoromethyl;
the phenyl and 6-member heteroaryl are substituted at the para position with trifluoromethyl;

X³ is a linker selected from the group consisting of:

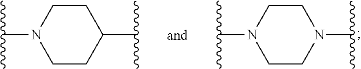 [SMILES: *C1CCN(*)CC1 ; "and" ; *N1CCN(*)CC1 ; ";"]

X⁵ is selected from the group consisting of a bond and —CH₂—;

X⁶ is —CH₂—, optionally substituted with C₁-C₃-alkyl;

X⁷ is selected from the group consisting of —C(O)— and —C(S); and

Z¹ is CH optionally substituted with a substituent selected from the group consisting of nitro and cyano.

Compounds encompassed by these embodiments include, for example:

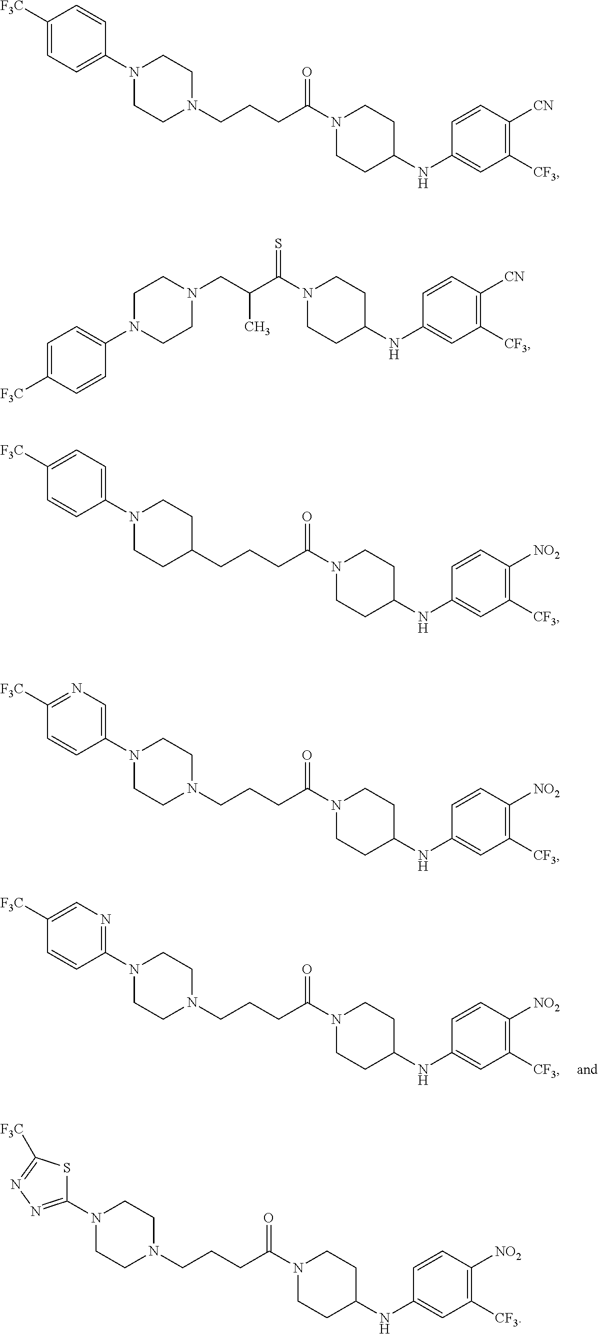

In some such embodiments of this invention, the compound is defined as corresponding in structure to the following formula:

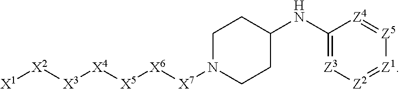

In some such embodiments of this invention, the compound is defined as corresponding in structure to the following formula:

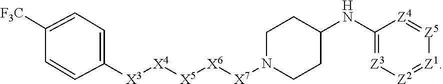

In some such embodiments of this invention, the compound is defined as corresponding in structure to the following formula:

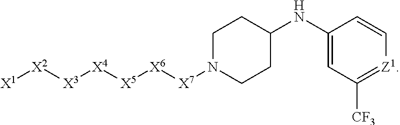

In some such embodiments of this invention, the compound is defined as corresponding in structure to the following formula:

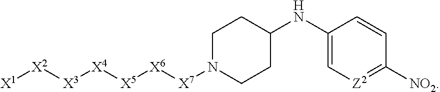

In some such embodiments of this invention, the compound is defined as corresponding in structure to the following formula:

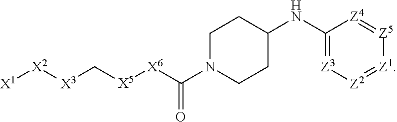

In some such embodiments of this invention, the compound is defined as corresponding in structure to the following formula:

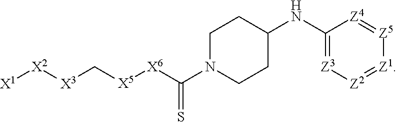

In some such embodiments of this invention, the compound is defined as corresponding in structure to the following formula:

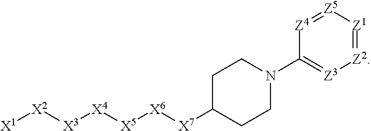

In some such embodiments of this invention, the compound is defined as corresponding in structure to the following formula:

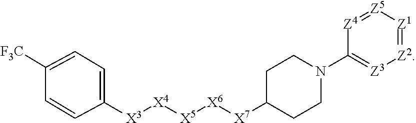

In some such embodiments of this invention, the compound is defined as corresponding in structure to the following formula:

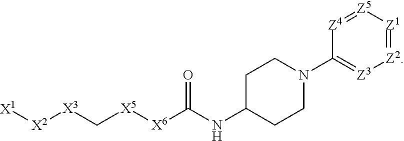

In some such embodiments of this invention, the compound is defined as corresponding in structure to the following formula:

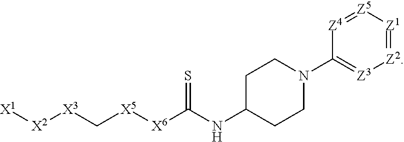

In some embodiments of this invention, the compound is defined as corresponding in structure to the following formula:

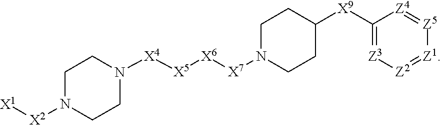

In some such embodiments of this invention, the compound is defined as corresponding in structure to the following formula:

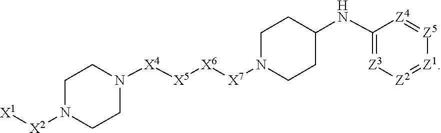

In some such embodiments of this invention, the compound is defined as corresponding in structure to the following formula:

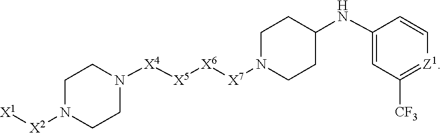

In other some embodiments of this invention, the compound is defined as corresponding in structure to the following formula:

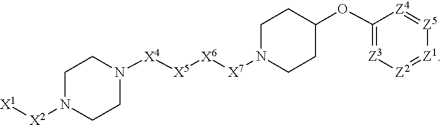

In some such embodiments of this invention, the compound is defined as corresponding in structure to the following formula:

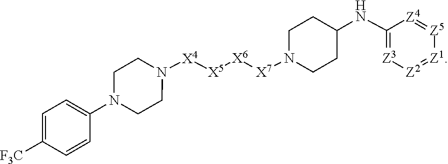

In some embodiments of this invention, the compound is defined as corresponding in structure to the following formula:

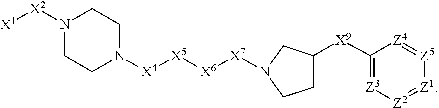

In some embodiments of this invention, the compound is defined as corresponding in structure to the following formula:

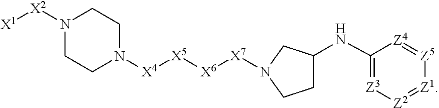

In some embodiments of this invention, the compound is defined as corresponding in structure to the following formula:

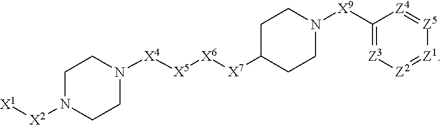

In some embodiments of this invention, the compound is defined as corresponding in structure to the following formula:

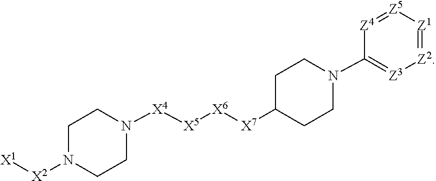

In some such embodiments of this invention, the compound is defined as corresponding in structure to the following formula:

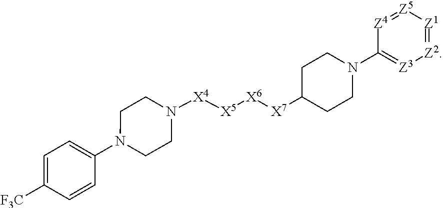

In some embodiments of this invention, the compound is defined as corresponding in structure to the following formula:

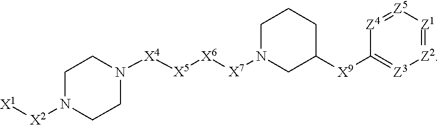

In some embodiments of this invention, the compound is defined as corresponding in structure to the following formula:

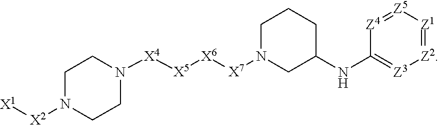

In some embodiments of this invention, the compound is defined as corresponding in structure to the following formula:

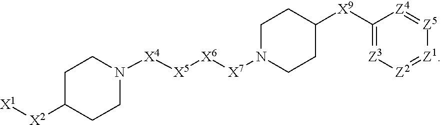

In some such embodiments of this invention, the compound is defined as corresponding in structure to the following formula:

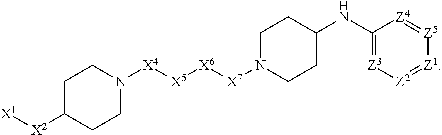

In other such embodiments of this invention, the compound is defined as corresponding in structure to the following formula:

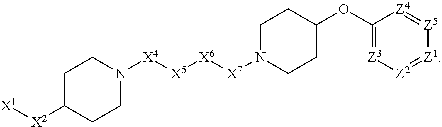

In some embodiments of this invention, the compound is defined as corresponding in structure to the following formula:

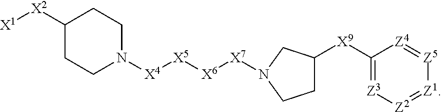

In some embodiments of this invention, the compound is defined as corresponding in structure to the following formula:

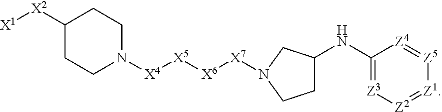

In some embodiments of this invention, the compound is defined as corresponding in structure to the following formula:

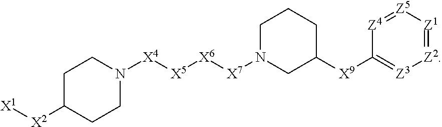

In some embodiments of this invention, the compound is defined as corresponding in structure to the following formula:

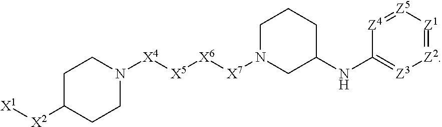

In some embodiments of this invention, the compound is defined as corresponding in structure to the following formula:

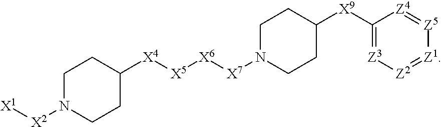

In some embodiments of this invention, the compound is defined as corresponding in structure to the following formula:

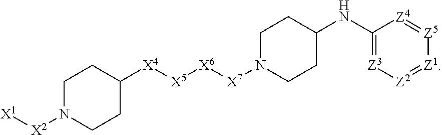

In some embodiments of this invention, the compound is defined as corresponding in structure to the following formula:

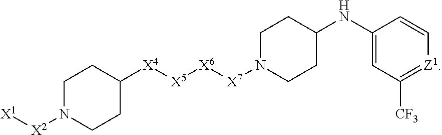

In some embodiments of this invention, the compound is defined as corresponding in structure to the following formula:

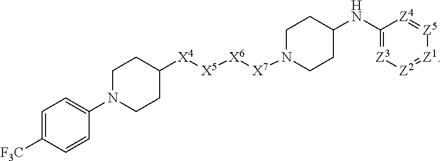

In some embodiments of this invention, the compound is defined as corresponding in structure to the following formula:

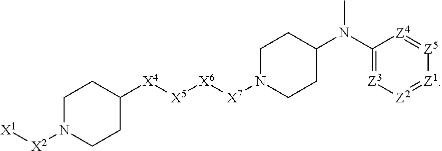

In some embodiments of this invention, the compound is defined as corresponding in structure to the following formula:

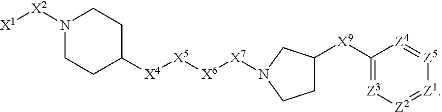

In some embodiments of this invention, the compound is defined as corresponding in structure to the following formula:

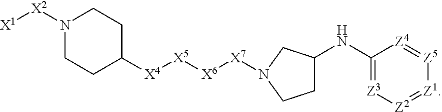

In some embodiments of this invention, the compound is defined as corresponding in structure to the following formula:

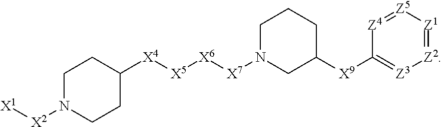

In some embodiments of this invention, the compound is defined as corresponding in structure to the following formula:

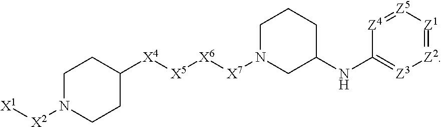

In some embodiments of this invention, the compound is defined as corresponding in structure to the following formula:

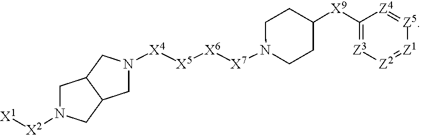

In some embodiments of this invention, the compound is defined as corresponding in structure to the following formula:

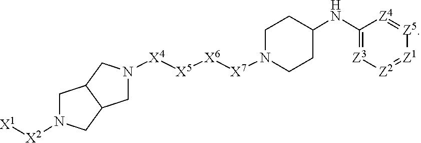

In some embodiments of this invention, the compound is defined as corresponding in structure to the following formula:

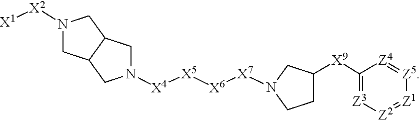

In some embodiments of this invention, the compound is defined as corresponding in structure to the following formula:

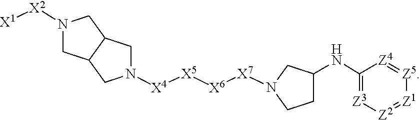

In some embodiments of this invention, the compound is defined as corresponding in structure to the following formula:

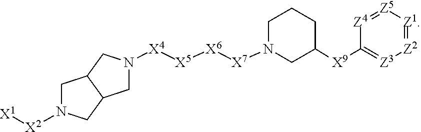

In some embodiments of this invention, the compound is defined as corresponding in structure to the following formula:

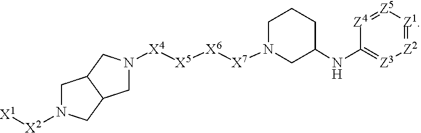

In some embodiments of this invention, the compound is defined as corresponding in structure to the following formula:

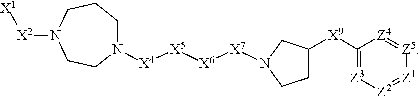

In some embodiments of this invention, the compound is defined as corresponding in structure to the following formula:

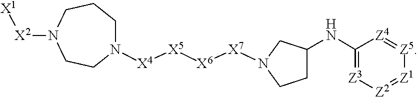

In some embodiments of this invention, the compound is defined as corresponding in structure to the following formula:

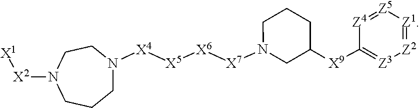

In some embodiments of this invention, the compound is defined as corresponding in structure to the following formula:

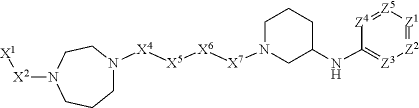

In some embodiments of this invention, the compound is defined as corresponding in structure to the following formula:

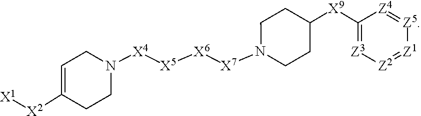

In some embodiments of this invention, the compound is defined as corresponding in structure to the following formula:

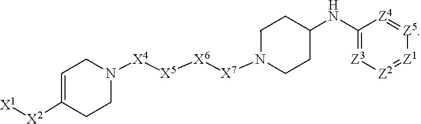

In some embodiments of this invention, the compound is defined as corresponding in structure to the following formula:

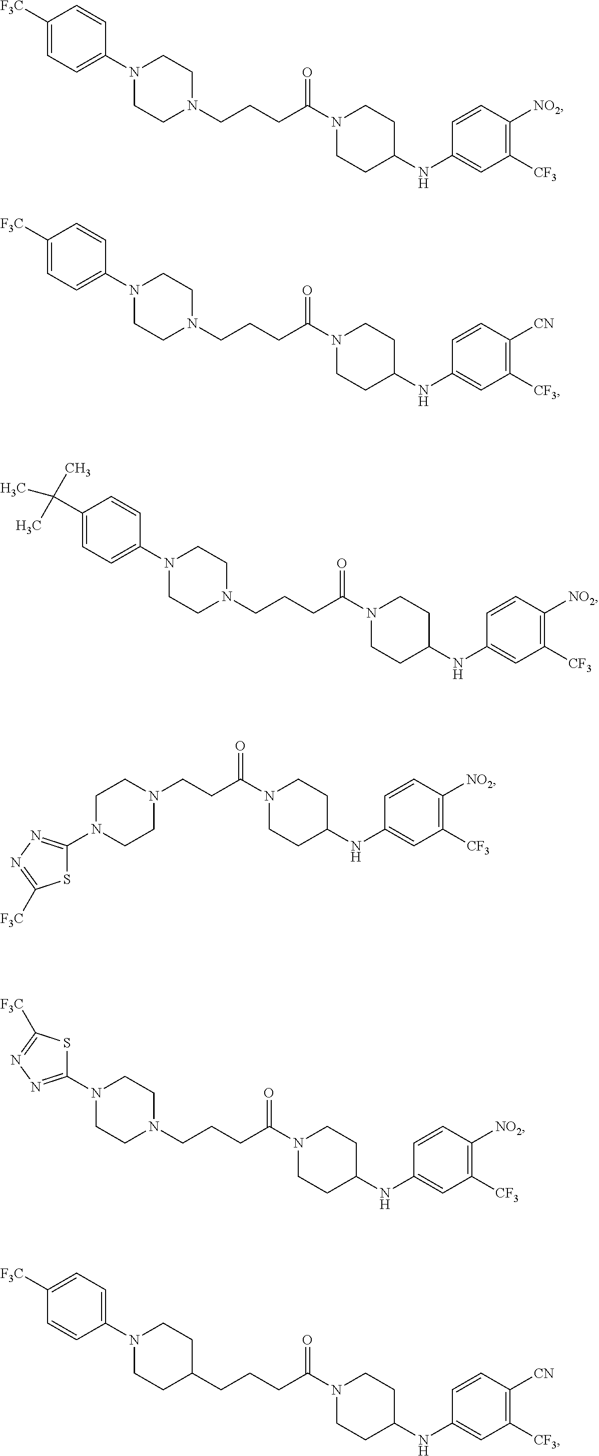 [SMILES: O=C(CCCN1CCN(c2ccc(C(F)(F)F)cc2)CC1)N1CCC(Nc2ccc([N+](=O)[O-])c(C(F)(F)F)c2)CC1 ; N#Cc1ccc(NC2CCN(C(=O)CCCN3CCN(c4ccc(C(F)(F)F)cc4)CC3)CC2)cc1C(F)(F)F ; CC(C)(C)c1ccc(N2CCN(CCCC(=O)N3CCC(Nc4ccc([N+](=O)[O-])c(C(F)(F)F)c4)CC3)CC2)cc1 ; O=C(CCN1CCN(c2nnc(C(F)(F)F)s2)CC1)N1CCC(Nc2ccc([N+](=O)[O-])c(C(F)(F)F)c2)CC1 ; O=C(CCCN1CCN(c2nnc(C(F)(F)F)s2)CC1)N1CCC(Nc2ccc([N+](=O)[O-])c(C(F)(F)F)c2)CC1 ; N#Cc1ccc(NC2CCN(C(=O)CCCC3CCN(c4ccc(C(F)(F)F)cc4)CC3)CC2)cc1C(F)(F)F]

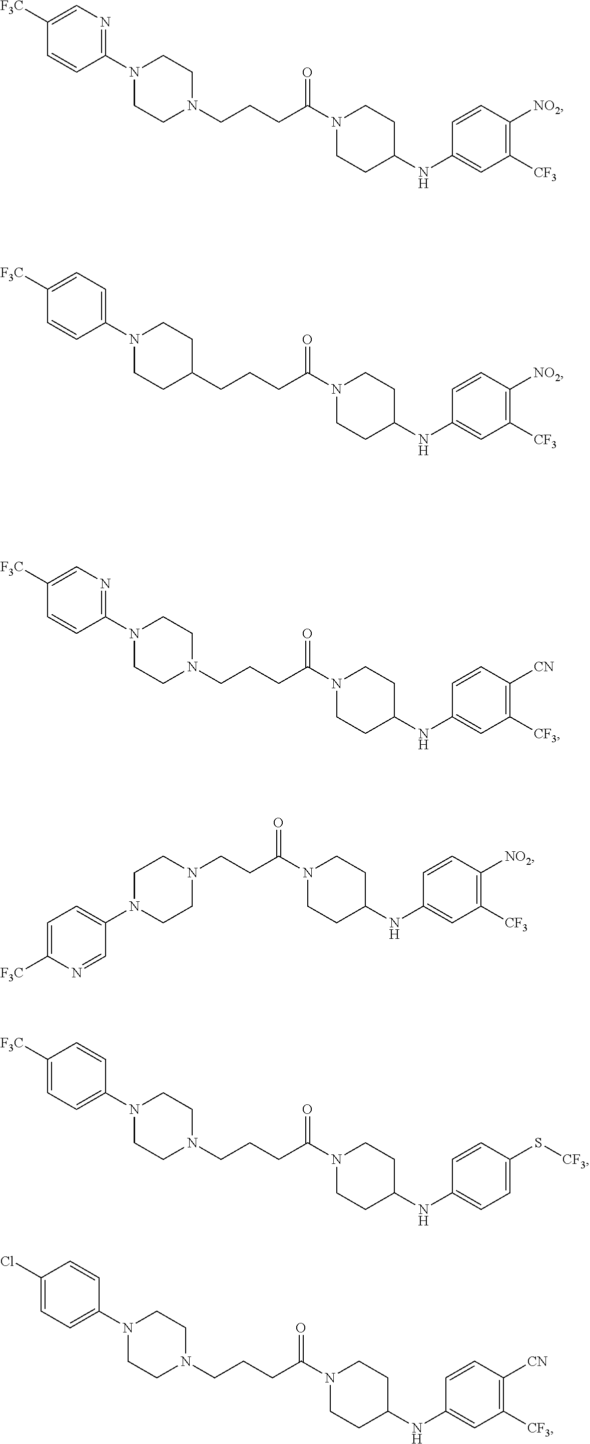 [SMILES: O=C(CCCN1CCN(c2ccc(C(F)(F)F)cn2)CC1)N1CCC(Nc2ccc([N+](=O)[O-])c(C(F)(F)F)c2)CC1 ; O=C(CCCC1CCN(c2ccc(C(F)(F)F)cc2)CC1)N1CCC(Nc2ccc([N+](=O)[O-])c(C(F)(F)F)c2)CC1 ; N#Cc1ccc(NC2CCN(C(=O)CCCN3CCN(c4ccc(C(F)(F)F)cn4)CC3)CC2)cc1C(F)(F)F ; O=C(CCN1CCN(c2ccc(C(F)(F)F)nc2)CC1)N1CCC(Nc2ccc([N+](=O)[O-])c(C(F)(F)F)c2)CC1 ; O=C(CCCN1CCN(c2ccc(C(F)(F)F)cc2)CC1)N1CCC(Nc2ccc(SC(F)(F)F)cc2)CC1 ; N#Cc1ccc(NC2CCN(C(=O)CCCN3CCN(c4ccc(Cl)cc4)CC3)CC2)cc1C(F)(F)F]

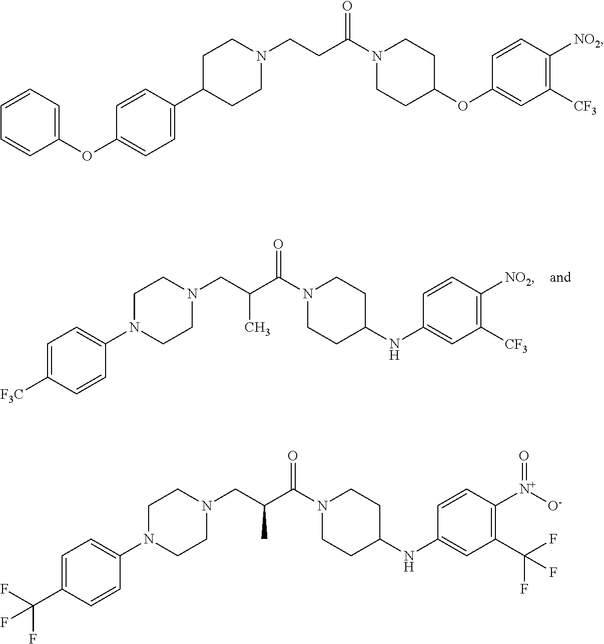 [SMILES: O=C(CCN1CCC(c2ccc(Oc3ccccc3)cc2)CC1)N1CCC(Oc2ccc([N+](=O)[O-])c(C(F)(F)F)c2)CC1 ; CC(CN1CCN(c2ccc(C(F)(F)F)cc2)CC1)C(=O)N1CCC(Nc2ccc([N+](=O)[O-])c(C(F)(F)F)c2)CC1 ; "and" ; C[C@@H](CN1CCN(c2ccc(C(F)(F)F)cc2)CC1)C(=O)N1CCC(Nc2ccc([N+](=O)[O-])c(C(F)(F)F)c2)CC1]

In other embodiments, the compound is selected from the group consisting of:

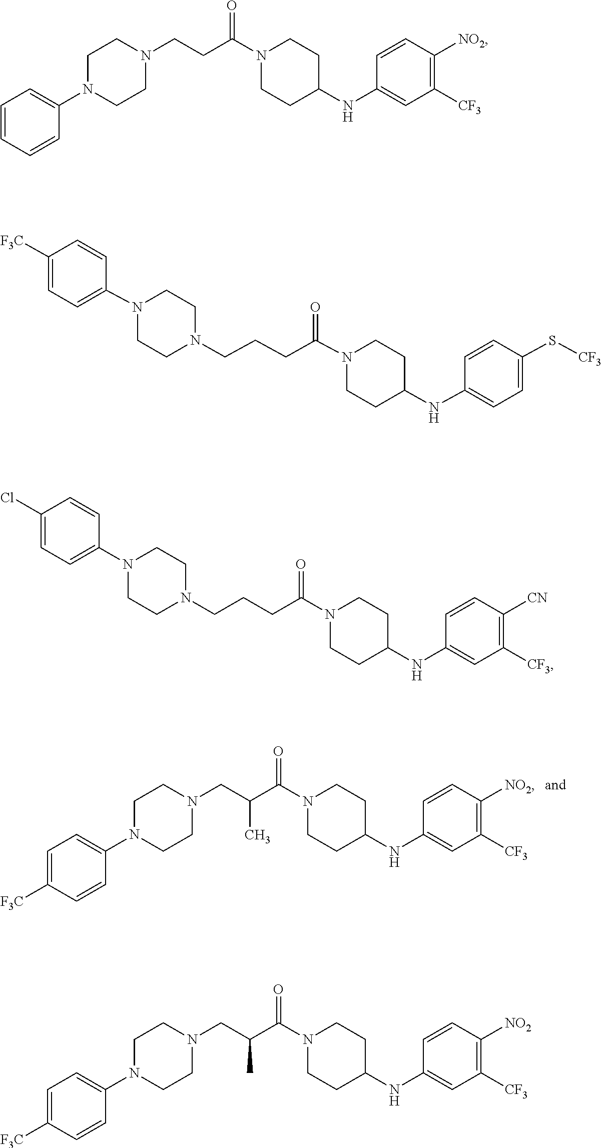 [SMILES: O=C(CCN1CCN(c2ccccc2)CC1)N1CCC(Nc2ccc([N+](=O)[O-])c(C(F)(F)F)c2)CC1 ; O=C(CCCN1CCN(c2ccc(C(F)(F)F)cc2)CC1)N1CCC(Nc2ccc(SC(F)(F)F)cc2)CC1 ; N#Cc1ccc(NC2CCN(C(=O)CCCN3CCN(c4ccc(Cl)cc4)CC3)CC2)cc1C(F)(F)F ; CC(CN1CCN(c2ccc(C(F)(F)F)cc2)CC1)C(=O)N1CCC(Nc2ccc([N+](=O)[O-])c(C(F)(F)F)c2)CC1 ; "and" ; C[C@@H](CN1CCN(c2ccc(C(F)(F)F)cc2)CC1)C(=O)N1CCC(Nc2ccc([N+](=O)[O-])c(C(F)(F)F)c2)CC1]

In other embodiments, the compound is selected from the group consisting of:

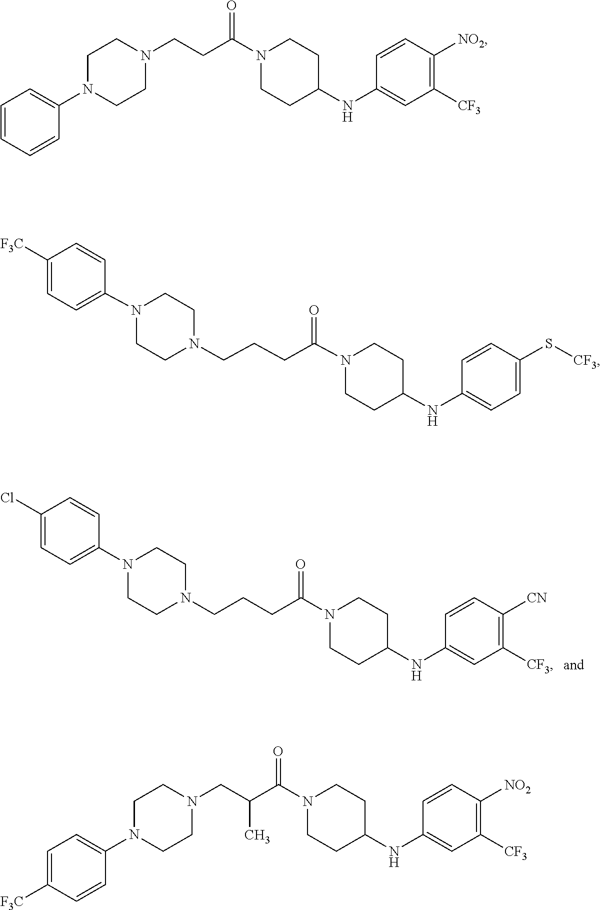 [SMILES: O=C(CCN1CCN(c2ccccc2)CC1)N1CCC(Nc2ccc([N+](=O)[O-])c(C(F)(F)F)c2)CC1 ; O=C(CCCN1CCN(c2ccc(C(F)(F)F)cc2)CC1)N1CCC(Nc2ccc(SC(F)(F)F)cc2)CC1 ; N#Cc1ccc(NC2CCN(C(=O)CCCN3CCN(c4ccc(Cl)cc4)CC3)CC2)cc1C(F)(F)F ; "and" ; CC(CN1CCN(c2ccc(C(F)(F)F)cc2)CC1)C(=O)N1CCC(Nc2ccc([N+](=O)[O-])c(C(F)(F)F)c2)CC1]

### N. Isomers

In some embodiments, a compound for use in the invention may have two or more conformational or geometric structures. For example, the following compound can have a cis or trans configuration:

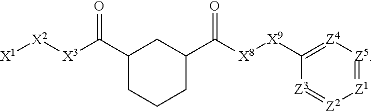

In some embodiments, this compound has the trans configuration such that the compound is encompassed by following formula:

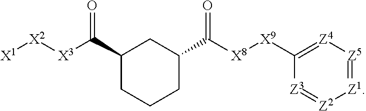

In other embodiments, the compound has the cis configuration such that the compound is encompassed by the following formula:

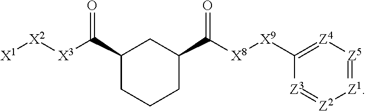

Unless otherwise stated, a compound structure that does not indicate a particular conformation is intended to encompass compositions of all the possible conformational isomers of the compound, as well as compositions comprising fewer than all the possible conformational isomers.

In some embodiments, a compound for use in the invention is a chiral compound. For example, the following compound can have an R or S configuration:

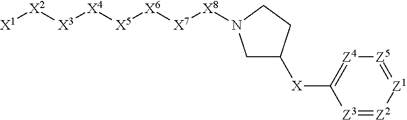

In some embodiments, this compound is one enantiomer such that the compound is encompassed by the following formula:

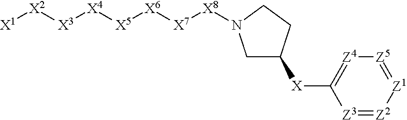

In some embodiments, this compound is the other enantiomer such that the compound is encompassed by the following formula:

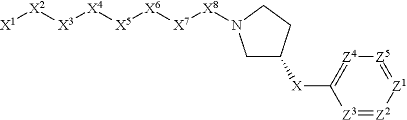

In some embodiments, the compound for use in the invention is a non-chiral compound.

Unless otherwise stated, a chiral compound structure that does not indicate a particular enantiomer is intended to encompass compositions of all possible enantiomers, diastereomers, and stereoisomers of the compound, as well as compositions comprising fewer than all the possible enantiomers, diastereomers, and stereoisomers, including racemic mixtures.

### II. Salts of Compounds for Use in the Invention

A salt of the above-described compounds may be advantageous due to one or more of the salt's physical properties, such as pharmaceutical stability in differing temperatures and humidities; crystalline properties; and/or a desirable solubility in water, oil, or other solvent. In some instances, a salt may be used as an aid in the isolation, purification, and/or resolution of the compound. Acid and base salts can typically be formed by, for example, mixing the compound with an acid or base, respectively, using various known methods in the art. To the extent a salt of the compound is intended to be administered in vivo (i.e., to an animal) for a therapeutic benefit, the salt preferably is pharmaceutically acceptable.

In general, an acid addition salt can be prepared by reacting a free base compound with an approximately stoichiometric amount of an inorganic or organic acid. Examples of often suitable inorganic acids for making pharmaceutically acceptable salts include hydrochloric, hydrobromic, hydroiodic, nitric, carbonic, sulfuric, and phosphoric acid. Examples of often suitable organic acids for making pharmaceutically acceptable salts generally include, for example, aliphatic, cycloaliphatic, aromatic, araliphatic, heterocyclic, carboxylic, and sulfonic classes of organic acids. Specific examples of often suitable organic acids include cholic, sorbic, lauric, acetic, trifluoroacetic, formic, propionic, succinic, glycolic, gluconic, digluconic, lactic, malic, tartaric acid, citric, ascorbic, glucuronic, maleic, fumaric, pyruvic, aspartic, glutamic, aryl carboxylic acid (e.g., benzoic), anthranilic acid, mesylic, stearic, salicylic, p-hydroxybenzoic, phenylacetic, mandelic, embonic (pamoic), alkylsulfonic (e.g., ethanesulfonic), arylsulfonic (e.g., benzenesulfonic), pantothenic, 2-hydroxyethanesulfonic, sulfanilic, cyclohexylaminosulfonic, β-hydroxybutyric, galactaric, galacturonic, adipic, alginic, butyric, camphoric, camphorsulfonic, cyclopentanepropionic, dodecylsulfic, glycoheptanoic, glycerophosphic, heptanoic, hexanoic, nicotinic, 2-naphthalesulfonic, oxalic, palmoic, pectinic, 3-phenylpropionic, picric, pivalic, thiocyanic, tosylic, and undecanoic acid. In some such embodiments, for example, the salt comprises a trifluoroacetate, mesylate, or tosylate salt. In other embodiments, the salt comprises a hydrochloric acid salt.

In general, a base addition salt can be prepared by reacting a free acid compound with an approximately stoichiometric amount of an inorganic or organic base. Examples of base addition salts may include, for example, metallic salts and organic salts. Metallic salts, for example, include alkali metal (group Ia) salts, alkaline earth metal (group IIa) salts, and other physiologically acceptable metal salts. Such salts may be made from aluminum, calcium, lithium, magnesium, potassium, sodium, and zinc. For example, a free acid compound may be mixed with sodium hydroxide to form such a base addition salt. Organic salts may be made from amines, such as trimethylamine, diethylamine, N,N′-dibenzylethylenediamine, chloroprocaine, ethanolamine, diethanolamine, ethylenediamine, meglumine (N-methylglucamine), and procaine. Basic nitrogen-containing groups may be quaternized with agents such as C₁-C₆-alkyl halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), arylalkyl halides (e.g., benzyl and phenethyl bromides), and others.

### III. Treatment Methods Using Compounds and Salts of this Invention

In accordance with this invention, it has been discovered that the compounds and salts thereof are particularly useful for treating infections with Dirofilaria immitis. It is contemplated that the compounds and salts of this invention may be used to treat a range of animals, especially mammals, for example wild animals such as wolves, coyotes, foxes and raccoons and companion animals such as dogs, cats and ferrets.

The compounds and salts of this invention may be administered orally. For example, the compound or salt may be added to the intended recipient's feed, either directly or as part of a premix. The compound or salt alternatively may be administered as, for example, a separate solid dosage form (e.g., a tablet, a hard or soft capsule, granules, powders, etc.), paste, or liquid dosage form (e.g., a solution, suspension, syrup, etc.).

A dosage form may comprise one or more suitable excipients. Such excipients generally include, for example, sweetening agents, flavoring agents, coloring agents, preservative agents, inert diluents (e.g., calcium carbonate, sodium carbonate, lactose, calcium phosphate, sodium phosphate, or kaolin), granulating and disintegrating agents (e.g., corn starch or alginic acid), binding agents (e.g., gelatin, acacia, or carboxymethyl cellulose), and lubricating agents (e.g., magnesium stearate, stearic acid, or talc). The compounds may be premixed with the excipients or provided as separate entities, e.g. to be mixed at the site of administration (depending i.a. on the type of excipients, desired stability, transport requirements, desired ease of use etc.). A solid dispersion of particular use may be based on a polymer or graft copolymer, e.g. of polyethylene glycol, polyvinyl caprolactam, polyvinyl acetate and/or combinations thereof, amenable to solid dispersion techniques such as hot melt extrusion, spray drying and top spray granulation. The polymer may serve as a carrier for the active compound for use according to the invention. In particular a mixture of such a compound (about 5 g) and of a graft copolymer amenable to solid dispersion techniques such as a polyvinyl caprolactam-polyvinyl acetate-polyethylene glycol graft copolymer (about 10 g) is homogenized for about 20 minutes. Extrusion of the powder mixture is then performed using an extrusion equipment preheated at about 200° C. The obtained extrudate is then cooled down to room temperature; is ground to a fine powder for about 30 minutes using a ball mill. About 12 g of powdered extrudate is finally isolated.

Liquid compositions will generally comprise a solvent, such as, for example, one or more of dimethylformamide, N,N-dimethylacetamide, pyrrolidone, N-methylpyrrolidone, polyethyleneglycol, diethyleneglycolmonoethyl ester, dimethylsulfoxide, and ethyl lactate. The solvent preferably has sufficient chemical properties and quantity to keep the compound or salt solubilized under normal storage conditions. In some instances, it may be desirable for the compositions to comprise one or more preservatives. The presence of a preservative may, for example, allow for the compositions to be stored for longer periods. Every excipient in the composition preferably is pharmaceutically acceptable.

It is contemplated that the compounds and salts of this invention may alternatively be administered via non-oral routes, such as rectally, via inhalation (e.g., via a mist or aerosol), transdermally (e.g., via a transdermal patch), or parenterally (e.g., subcutaneous injection, intravenous injection, intramuscular injection, etc.).

In general, the compositions of this invention are administered in a dosage form that provides a therapeutically effective amount of the compound or salt to the site of infection. A “therapeutically effective amount” is an amount that is sufficient to prevent, ameliorate, suppress, or eradicate a target pathogen(s) infection (which may be at any stage of the pathogen), which is equal to “treating an infection with the target pathogen”. In particular for Dirofilaria immitis, by treating the infection, heartworm disease, i.e. any disorder arising from an infection with Dirofilaria immitis, is treated (i.e. prevented, ameliorated, suppressed or cured). Generally, the therapeutically effective amount is defined as the amount necessary to achieve a concentration efficacious to control the target pathogen(s) at the site of infection. The concentration at the site of infection is preferably at least equal to the MIC100 level (minimum inhibitory concentration, i.e., the concentration that inhibits the motility of 100% of the target pathogen) of the compound or salt thereof for the target pathogen. To the extent the compound or salt is administered with another active ingredient(s) (e.g., one or more other anthelmintics), the dosage preferably comprises an amount of the compound or salt that, together with the amount of other active ingredient(s), constitutes a therapeutically effective amount.

A single administration of the compound or salt may be sufficient to treat an infection with Dirofilaria immitis. Although such a single dose is typically preferred, it is contemplated that multiple doses can be used. When the compound or salt is orally administered, the total dose to treat an infection is generally greater than about 0.01 mg/kg (i.e., milligram of compound or salt per kilogram body weight). In some such embodiments, the total dose is from about 0.01 to about 100 mg/kg, from about 0.01 to about 50 mg/kg, from about 0.1 to about 25 mg/kg, or from about 1 to about 20 mg/kg. For dogs, for example, the dose is generally from about 1 to about 15 mg/kg, from about 8 to about 12 mg/kg, or about 10 mg/kg. The same dose range may be suitable for other routes of administration. For example, in some embodiments, the same dose range is used for subcutaneous administration. The desired dose, however, may be less in some instances where the compound or salt is administered parenterally, particularly intravenously. For example, in some such embodiments, the dose is from about 0.01 to about 50 mg/kg, from about 0.01 to about 15 mg/kg, or from about 0.1 to about 10 mg/kg. For dogs, for example, a suitable intravenous dose may be from about 0.01 to about 10 mg/kg, from about 0.1 to about 5 mg/kg, or about 1 mg/kg.

If the compound or salt is administered parenterally via an injection, the concentration of the compound or salt in the dosage form preferably is sufficient to provide the desired therapeutically effective amount of the compound or salt in a volume that is acceptable for parenteral administration.

Factors affecting the preferred dosage may include, for example, the type (e.g., species and breed), age, size, sex, diet, activity, and condition of the intended recipient; the administration route; pharmacological considerations, such as the activity, efficacy, pharmacokinetic, and toxicology profiles of the particular composition administered; and whether the compound or salt is being administered as part of a combination of active ingredients. Thus, the preferred amount of the compound or salt can vary, and, therefore, can deviate from the typical dosages set forth above. Determining such dosage adjustments is generally within the skill of those in the art.

This invention is also directed to combinations which are useful for pharmaceutical compositions comprising a) one or more compounds for use according to the invention with b) one or more active compounds which differ in structure from component a). The active compounds b) are preferably anthelmintic compounds, more preferably selected from the group consisting of avermectins (e.g., ivermectin, selamectin, doramectin, abamectin, and eprinomectin); milbemycins (moxidectin and milbemycin oxime); pro-benzimidazoles (e.g., febantel, netobimin, and thiophanate); benzimidazole derivatives, such as a thiazole benzimidazole derivative (e.g., thiabendazole and cambendazole) or a carbamate benzimidazole derivatives (e.g., fenbendazole, albendazole (oxide), mebendazole, oxfendazole, parbendazole, oxibendazole, flubendazole, and triclabendazole); an imidazothiazoles (e.g., levamisole and tetramisole); a tetrahydropyrimidine (morantel and pyrantel), organophosphates (e.g., trichlorphon, haloxon, dichlorvos, and naphthalophos); salicylanilides (e.g., closantel, oxyclozanide, rafoxanide, and niclosamide); nitrophenolic compounds (e.g., nitroxynil and nitroscanate); benzoenedisulphonamides (e.g., clorsulon); pyrazinaisoquinolines (e.g., praziquantel and epsiprantel); heterocyclic compounds (e.g., piperazine, diethylcarbamazine, dichlorophen, and phenothiazine); arsenicals (e.g., thiacetarsamide, melorsamine, and arsenamide); cyclooctadepsipeptides (e.g., emodepside); paraherquamides (e.g. derquantel); amino-acetonitrile compounds (e.g. monepantel, AAD 1566); and amidine compounds (e.g., amidantel and tribendimidin) (including all pharmaceutically acceptable forms, such as salts).

In the contemplated combination therapies, the compounds for use according to this invention may be administered before, simultaneously, and/or after the other active ingredient(s). In addition, the compounds for use according to this invention may be administered in the same composition as the other active ingredient(s) and/or in a separate compositions from the other active ingredient(s). Further, the compounds for use according to this invention and other active ingredient(s) may be administered via the same and/or different routes of administration.

### EXAMPLES

The following examples are merely illustrative, and not limiting to the remainder of this disclosure in any way.

### Example 1. Protocols for Analyzing Compounds Prepared for Use with this Invention

Applicants prepared a plethora of compounds for use according to the invention. The identities and purities were characterized and verified using various analytical high performance liquid chromatography (“HPLC”) and mass spectroscopy (“MS”) protocols. These protocols are discussed below.

### System I

In some instances, the compound analysis was conducted using an HPLC/MSD 1100 (Agilent, Santa Clara, Calif., USA) having a binary pump (G1312A) with a degasser (G1379A), a well plate sampler (G1367A), a column oven (G1316A), a diode array detector (G1315B), a mass detector (G1946D SL) with an ESI-source, and an evaporating light detector (Sedex 75). Four different columns and detection methods were used with this system:

### Protocol I-A

The column used for this protocol was a Zorbax SB-C18 (Agilent), having a 4.6 mm diameter, a 30 mm length, and 3.5 μm packing. The column was operated at 30° C. (ambient temperature). The injection volume was 5.0 μL, the flow rate was 1.0 ml/min, and the run time was 8 min (equilibration included). Two eluents were used with the following gradients:

| | Solvent A (%) | Solvent B (%) |
|---|---|---|
| Time | water/formic acid, | acetonitrile/formic acid, |
| (min) | 99.9/0.1 (v/v) | 99.9/0.1 (v/v) |
| 0.0 | 90 | 10 |
| 0.2 | 90 | 10 |
| 4.2 | 2 | 98 |
| 5.5 | 2 | 98 |

The samples were diluted in a 1:1 mixture of solvents A and B before analysis. The detection methods were UV at 210 and 254 nm; ESI/MS (100-1000 m/z), positive ions; and ELSD (Sedex 75).

### Protocol I-B

The column used for this protocol was an Atlantis dC18 (Waters, Milford, Mass., USA), having a 4.6 mm diameter, a 50 mm length, and 3 μm packing. The column was operated at 30° C. The injection volume was 2.0 μL, the flow rate was 1.0 ml/min, and the run time was 10 min (equilibration included). Two eluents were used with the following gradients:

| | Solvent A (%) | Solvent B (%) |
|---|---|---|
| Time | water/formic acid, | acetonitrile/formic acid, |
| (min) | 99.9/0.1 (v/v) | 99.9/0.1 (v/v) |
| 0.0 | 95 | 5 |
| 1.0 | 95 | 5 |
| 5.0 | 2 | 98 |
| 7.0 | 2 | 98 |

The samples were diluted in a 1:1 mixture of solvents A and B before analysis. The detection methods were UV at 210 and 254 nm; ESI/MS (100-1000 m/z), positive ions; and ELSD (Sedex 75).

### Protocol I-C

The column used for this protocol was an Atlantis dC18, having a 4.6 mm diameter, a 50 mm length, and 3 μm packing. The column was operated at 30° C. The injection volume was 2.0 μL, the flow rate was 1.5 ml/min, and the run time was 6 min (equilibration included). Two eluents were used with the following gradients:

| | Solvent A (%) | Solvent B (%) |
|---|---|---|
| Time | water/formic acid, | acetonitrile/formic acid, |
| (min) | 99.9/0.1 (v/v) | 99.9/0.1 (v/v) |
| 0.0 | 90 | 10 |
| 0.5 | 90 | 10 |
| 3.0 | 2 | 98 |
| 4.0 | 2 | 98 |

The samples were diluted in a 1:1 mixture of solvents A and B before analysis. The detection methods were UV at 210 and 254 nm; ESI/MS (85-1000 m/z), positive ions; and ELSD (Sedex 75).

### Protocol I-D

The column used for this protocol was a Chromolith Fast Gradient, RP-18e, 2 mm diameter and a 50 mm length. The column was operated at 35° C. The injection volume was 1.0 μL, the flow rate was 1.2 mL/min, and the run time was 3.5 min (equilibration included). Two eluents were used with the following gradients:

| | Solvent A (%) | Solvent B (%) |
|---|---|---|
| Time | water/formic acid, | acetonitrile/formic acid, |
| (min) | 99.9/0.1 (v/v) | 99.9/0.1 (v/v) |
| 0.0 | 90 | 10 |
| 2.0 | 0 | 100 |
| 2.7 | 0 | 100 |
| 3.0 | 90 | 10 |

The samples were diluted in a 1:1 mixture of A and B before analysis. The diction methods were UV at 210 and 254 nm; ESI/MS (100-1000 m/z), positive ions; and ELSD (Sedex 75).

### Protocol I-E

The column used for this protocol was a Chromolith Fast Gradient, RP-18e, 2 mm diameter and a 50 mm length. The column was operated at 35° C. The injection volume was 1.0 μL, the flow rate was 1.2 mL/min, and the run time was 3.5 min (equilibration included). Two eluents were used with the following gradients:

| | Solvent A (%) | Solvent B (%) |
|---|---|---|
| Time | water/formic acid, | acetonitrile/formic acid, |
| (min) | 99.9/0.1 (v/v) | 99.9/0.1 (v/v) |
| 0.0 | 98 | 2 |
| 2.0 | 2 | 98 |
| 2.5 | 2 | 98 |
| 3.0 | 98 | 2 |

The samples were diluted in a 1:1 mixture of A and B before analysis. The detection methods were UV at 210 and 254 nm; ESI/MS (100-1000 m/z), positive ions; and ELSD (Sedex 75).

### System II

In some instances, the compound analysis was conducted using an LC/MSD Trap 1100 (Agilent, Santa Clara, Calif., USA) having a binary pump (G1312A) with a degasser (G1379A), a well plate sampler (G1367A), a column oven (G1316A), a diode array detector (G1315B), a mass detector (G2445D SL) with an APCI-source, and an evaporating light detector (Alltech ELSD2000). Three different columns and detection methods were used with this system:

### Protocol II-A

The column used for this protocol was a Zorbax SB-C18 (Agilent), having a 4.6 mm diameter, a 30 mm length, and 3.5 μm packing. The column was operated at 30° C. The injection volume was 5.0 μL, the flow rate was 1.0 ml/min, and the run time was 8 min (equilibration included). Two eluents were used with the following gradients:

| | Solvent A (%) | Solvent B (%) |
|---|---|---|
| Time | water/formic acid, | acetonitrile/formic acid, |
| (min) | 99.9/0.1 (v/v) | 99.9/0.1 (v/v) |
| 0.0 | 90 | 10 |
| 0.2 | 90 | 10 |
| 4.2 | 2 | 98 |
| 5.5 | 2 | 98 |

The samples were diluted in a 1:1 mixture of solvents A and B before analysis. The detection methods were UV at 210 and 254 nm; and APCI/MS (80-1000 m/z), positive ions.

### Protocol II-B

The column used for this protocol was an XBridge C18 (Waters), having a 4.6 mm diameter, a 50 mm length, and 2.5 μm packing. The column was operated at 40° C. The injection volume was 2.0 μL, the flow rate was 1.0 ml/min, and the run time was 10 min (equilibration included). Two eluents were used with the following gradients:

| | Solvent A (%) | |
|---|---|---|
| Time | water/ammonia, | Solvent B (%) |
| (min) | 99.9/0.1 (v/v) | acetonitrile |
| 0.0 | 75 | 25 |
| 5.0 | 0 | 100 |
| 7.0 | 0 | 100 |
| 7.5 | 75 | 25 |

The samples were diluted in a 1:1 mixture of solvents A and B before analysis. The detection methods were UV at 254 and 210 nm; and APCI/MS (100-1500 m/z), positive ions.

### Protocol II-C

The column used for this protocol was an Atlantis dC18 (Waters), having a 4.6 mm diameter, a 150 mm length, and 3 μm packing. The column was operated at 40° C. The injection volume was 5.0 μL, the flow rate was 1.0 ml/min, and the run time was 16 min (equilibration included). Two eluents were used with the following gradients:

| | Solvent A (%) | Solvent B (%) |
|---|---|---|
| Time | water/formic acid, | acetonitrile/formic acid, |
| (min) | 99.9/0.1 (v/v) | 99.9/0.1 (v/v) |
| 0.0 | 98 | 2 |
| 10 | 0 | 100 |
| 12 | 0 | 100 |
| 13 | 98 | 2 |

The samples were diluted in a 1:1 mixture of solvents A and B before analysis. The detection methods were UV at 254 and 210 nm; and APCI/MS (100-1000 m/z), positive ions.

### Protocol II-D

The column used for this protocol was an Atlantis dC18 (Waters), having a 4.6 mm diameter, a 50 mm length, and 3 μm packing. The column was operated at 40° C. The injection volume was 5.0 μL, the flow rate was 1.0 ml/min, and the run time was 8 min (equilibration included). Two eluents were used with the following gradients:

| | Solvent A (%) | Solvent B (%) |
|---|---|---|
| Time | water/formic acid, | acetonitrile/formic acid, |
| (min) | 99.9/0.1 (v/v) | 99.9/0.1 (v/v) |
| 0.0 | 90 | 10 |
| 10 | 0 | 100 |
| 12 | 0 | 100 |
| 13 | 90 | 10 |

The samples were diluted in a 1:1 mixture of solvents A and B before analysis. The detection methods were UV at 254 and 210 nm; and APCI/MS (100-1000 m/z), positive ions

### Protocol II-E

The column used for this protocol was a Phenomenex (Gemini), having a 4.6 mm diameter, a 150 mm length, and 5 μm packing. The column was operated at 35° C. The injection volume was 1.0 μL, the flow rate was 1.0 ml/min. Two eluents were used with the following gradients:

| | Solvent A (%) | Solvent B (%) |
|---|---|---|
| Time | 10 mM formic acid/ | 10 mM formic acid/ |
| (min) | acetonitrile | water |
| 0.0 | 2 | 98 |
| 10.5 | 98 | 2 |
| 18 | 98 | 2 |

The samples were diluted in a 1:1 mixture of solvents A and B before analysis. The detection methods were UV at 320 and 220 nm; and ESI/MS (100-800 m/z), positive and negative ions.

### Protocol II-F

The column used for this protocol was a Phenomenex (Gemini), having a 4.6 mm diameter, a 150 mm length, and 5 μm packing. The column was operated at 35° C. The injection volume was 1.0 μL, the flow rate was 1.0 ml/min. Two eluents were used with the following gradients:

| | Solvent A (%) | Solvent B (%) |
|---|---|---|
| Time | 10 mM ammonia/ | 10 mM ammonia/ |
| (min) | acetonitrile | water |
| 0.0 | 2 | 98 |
| 10.5 | 98 | 2 |
| 18 | 98 | 2 |

The samples were diluted in a 1:1 mixture of solvents A and B before analysis. The detection methods were UV at 320 and 220 nm; and ESI/MS (100-800 m/z), positive and negative ions.

### Exemplified Compounds

Compounds or salts thereof for use in the present invention are generally described in WO2010/115688. The examples 2 to 168 of WO2010/115688 (pages 120-223) are incorporated in this application as examples of compounds for use in the present invention, as well as methods of preparation thereof. The same is true for examples 169 through 1036 which are exemplified in table II of WO2010/115688 (page 223-318).

Additional examples of compounds for use in the present invention are described here beneath. Example 1037 is made as follows:

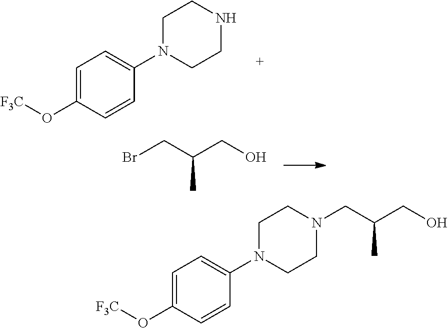

1-[4-(trifluoromethoxy)phenyl]piperazine (40 g; 162 mmol), (2R)-3-bromo-2-methyl-propan-1-ol (26.4 g; 166 mmol) and triethylamine (45.3 mL; 325 mmol) are dissolved in ethanol (350 mL) and the resulting mixture is stirred at reflux over the night. After cooling to room temperature, the reaction mixture is filtered over Celite and the filtrate is concentrated under reduced pressure. The obtained residue is dissolved in dichloromethane (300 mL) and washed twice with water (200 mL each). The organic phase is separated, dried over sodium sulfate, filtered and concentrated under reduced pressure. The crude product is purified by recrystallization from an ethanol-water mixture to afford after drying the desired product in pure form (31 g; 97 mmol).

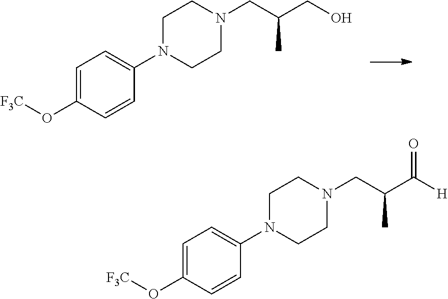 [SMILES: C[C@H](CO)CN1CCN(c2ccc(OC(F)(F)F)cc2)CC1 ; C[C@H](C=O)CN1CCN(c2ccc(OC(F)(F)F)cc2)CC1]

A 2 M solution in dichloromethane of oxalyl chloride (75 mL; 150 mmol) is diluted with dichloromethane (200 mL) and is cooled to −75° C. Dimethylsulfoxide (14.3 mL; 201 mmol) is added followed by a solution of (2S)-2-methyl-3-[4-[4-(trifluoromethoxy)phenyl]piperazin-1-yl]propan-1-ol (31.5 g; 100 mmol) in dichloromethane (250 mL). The reaction mixture is stirred at −75° C. for 45 min and is then allowed to reach room temperature. After 10 min at room temperature, water (500 mL) is added, the organic phase is separated and washed twice with water (250 mL each). After drying over sodium sulfate, concentration of the organic phase under reduced pressure affords the desired aldehyde (31 g; 100 mmol) as a crude product which is engaged as such in the next step.

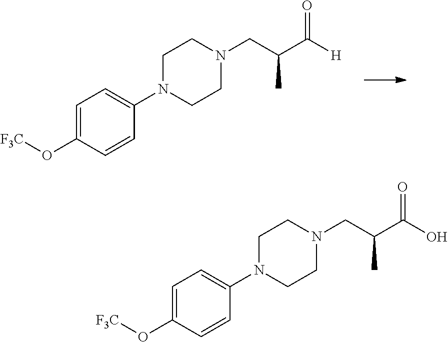 [SMILES: C[C@H](C=O)CN1CCN(c2ccc(OC(F)(F)F)cc2)CC1 ; C[C@@H](CN1CCN(c2ccc(OC(F)(F)F)cc2)CC1)C(=O)O]

(2S)-2-methyl-3-[4-[4-(trifluoromethoxy)phenyl]piperazin-1-yl]propanal (31.4 g; 99 mmol) is suspended in a mixture of tert-butanol (480 mL) and water (120 mL). 2-Methyl-butene (348 g; 4.96 mol) is added and the suspension is stirred at room temperature until a solution is obtained. Sodium dihydrogen phosphate (23.8 g; 199 mmol) is added to the solution at 5° C. and sodium chlorite (16.8 g; 149 mmol) is added in two equal portions. The reaction mixture is allowed to reach room temperature and is stirred for 2.5 h. The resulting suspension is filtered, the precipitate is washed twice with water (100 mL each) and is dried under reduced pressure at 50° C. to afford the desired product (20.5 g; 62 mmol).

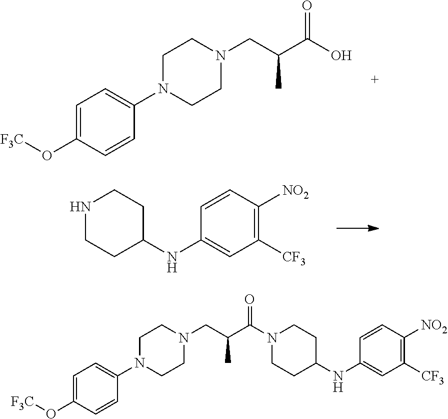

(2S)-2-Methyl-3-[4-[4-(trifluoromethoxy)phenyl]piperazin-1-yl]propanoic acid (10 g; 30 mmol) is suspended in dichloromethane (300 mL); O-Benzotriazole-N,N,N′,N′-tetramethyl-uronium-hexafluoro-phosphate (11.6 g; 30 mmol) and diisopropylethylamine (10.5 mL, 60 mmol) are added and the resulting mixture is stirred for 20 min at room temperature. N-[4-nitro-3-(trifluoromethyl)phenyl]piperidin-4-amine (9.6 g; 33 mmol) is added and the resulting solution is stirred for 4 h. The reaction mixture is then sequentially washed with aq. 1 M NaOH, 0.5 N HCl, water and brine (250 mL each). The organic phase is concentrated to afford the desired product as a crude. Precipitation from a mixture of dichloromethane and n-pentane yields the desired product is pure form (14.6 g; 24 mmol). The structure of this compound 1037 was confirmed using Protocol I-E. Calculated mass=603; observed mass=603; HPLC retention time=1.85 min.

Example 1038 is made as follows:

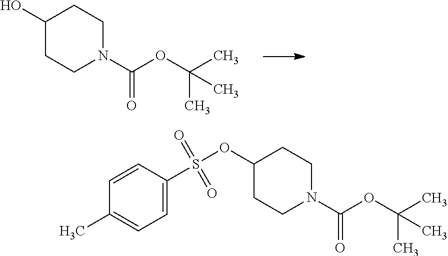

Tosyl chloride (11.8 g, 62 mmol) and tert-butyl 4-hydroxypiperidine-1-carboxylate (10 g, 50 mmol) are dissolved in pyridine (50 mL) and are stirred at room temperature until complete conversion is observed. The reaction mixture is concentrated under reduced pressure. The obtained residue is taken up in dichloromethane (200 mL), the organic layer is washed with water (2×70 mL), dried over magnesium sulfate, filtered and concentrated under reduced pressure. The pure desired product is obtained after recrystallisation of the crude product from n-heptane (15.1 g, 43 mmol).

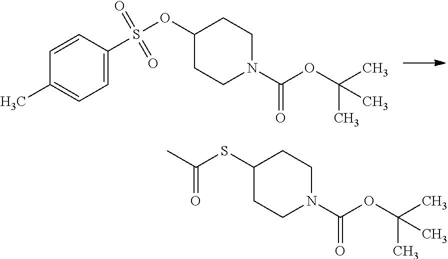

tert-Butyl 4-(p-tolylsulfonyloxy)piperidine-1-carboxylate (15.1 g, 43 mmol) and potassium thioacetate (23.5 g, 206 mmol) are dissolved in dimethylformamide (100 mL) and the resulting mixture is stirred at 50° C. for 5 h. The reaction mixture is cooled down to room temperature and is diluted with ethyl acetate (500 mL). The organic phase is washed with water (3×150 mL), is dried over magnesium sulfate, filtered and concentrated under reduced pressure. The crude residue is purified by column chromatography on silica gel using an dichloromethane gradient in hexane (from 50 to 100%). The fractions of interest are combined and concentrated under reduced pressure to afford the desired product.

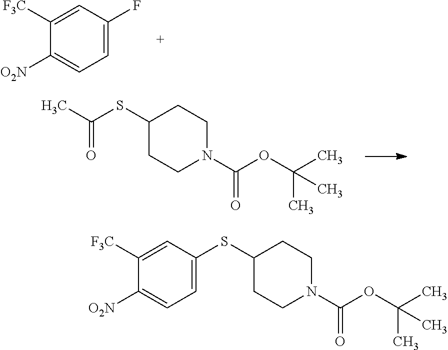

4-Fluoro-1-nitro-2-(trifluoromethyl)benzene (629 mg, 3 mmol), tert-butyl 4-acetylsulfanylpiperidine-1-carboxylate (900 mg, 3.5 mmol) and potassium carbonate (1.3 g, 9.4 mmol) are dissolved in a 2 to 10 mixture of water and acetonitrile (12 mL). The resulting mixture is stirred at 100° C. for 4 h. After cooling to room temperature, ethyl acetate (50 mL) is added. The organic layer is extracted with water (2×10 mL), dried over magnesium sulfate, filtered and concentrated under reduced pressure. The pure desired product is obtained after filtration over a pad of silica gel eluted with dichloromethane.

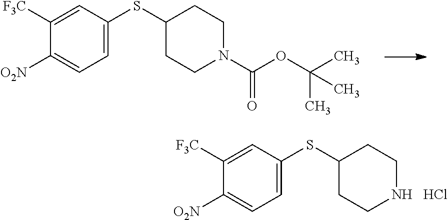

A solution of trifluoroacetic acid (30%) in dichloromethane (6 mL) is added to tert-butyl 4-[4-nitro-3-(trifluoromethyl)phenyl]sulfanylpiperidine-1-carboxylate (940 mg, 2.3 mmol) dissolved in a minimum volume of dichloromethane. The resulting mixture is stirred at room temperature for 20 min and is concentrated under reduced pressure. The residue is taken up in a 4 M solution of hydrochloric acid in dioxane. The precipitate formed in filtered off, is rinsed with diethyl ether (3×10 mL) and is dried in a vacuum oven to afford the desired product in pure form (728 mg, 2.1 mmol).

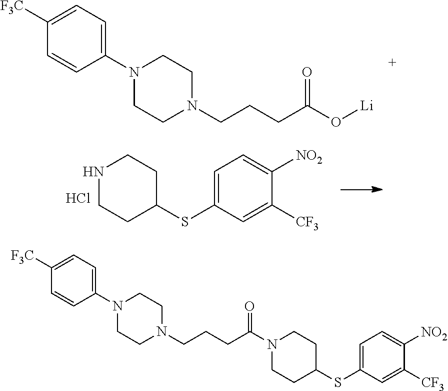

4-[4-[4-(trifluoromethyl)phenyl]piperazin-1-yl]butanoyloxylithium (0.05 mmol) and O-Benzotriazole-N,N,N′,N′-tetramethyl-uronium-hexafluoro-phosphate (0.05 mmol) are dissolved in a 7 to 3 mixture of tetrahydrofuran and dimethylformamide (1 mL). A solution of 4-[4-nitro-3-(trifluoromethyl)phenyl]sulfanylpiperidine hydrochloride (0.05 mmol) and of diisopropylethylamine (0.10 mmol) is added and the resulting mixture is stirred for 1 h at room temperature. The reaction mixture is concentrated under reduced pressure and is purified by preparative HPLC. The desired product is obtained in pure form as a solid (23 mg, 0.04 mmol). Its structure is confirmed using Protocol I-E. Calculated mass=587; observed mass=588; HPLC retention time=1.58 min.

### Example 1039 Determining Activity Against Dirofilaria immitis

Microfilariae recovered from Dirofilaria immitis infected dogs were plated in 96-well plates under sterile conditions. L3 larvae of Dirofilaria immitis were recovered from infected mosquitoes and allowed to molt into L4 stages required for compound testing. L4 larvae were plated in 96-well plates under sterile conditions. DMSO solutions of the compounds were added into parasite-containing plates. After compound addition, parasites were incubated for 3 days prior to assessment of viability. Microfilaricidal activity is reported as a half maximal effective concentration (EC₅₀). Effects on L4 larvae are reported as the lowest doses that result in complete loss of motility (MIC₁₀₀).

Compounds according to examples 1037, 156 (see WO2010/115688), 153 (see WO2010/115688), 64 (see WO2010/115688) and 48 (see WO2010/115688) exhibited EC₅₀ values of less than 10 μM against Dirofilaria immitis microfilariae. Compounds according to examples 1038, 942 (see WO2010/115688), 697 (see WO2010/115688), 689 (see WO2010/115688), 539 (see WO2010/115688), 444 (see WO2010/115688), 416 (see WO2010/115688), 157 (see WO2010/115688), 151 (see WO2010/115688), 141 (see WO2010/115688), 134 (see WO2010/115688), 89 (see WO2010/115688), 68 (see WO2010/115688), 54 (see WO2010/115688), 45 (see WO2010/115688), 33 (see WO2010/115688), 17 (see WO2010/115688), 12 (see WO2010/115688) and 7 (see WO2010/115688) exhibited EC₅₀ values of less than 5 μM against Dirofilaria immitis microfilariae.
Compounds according to example 1038, 157 (see WO2010/115688), 156 (see WO2010/115688), 134 (see WO2010/115688), 68 (see WO2010/115688), 64 (see WO2010/115688) and 45 (see WO2010/115688) exhibited MIC₁₀₀ values of less than 10 μM against L4 larvae of Dirofilaria immitis. Compounds according to example 1037, 942 (see WO2010/115688), 697 (see WO2010/115688), 689 (see WO2010/115688), 539 (see WO2010/115688), 444 (see WO2010/115688), 416 (see WO2010/115688), 153 (see WO2010/115688), 151 (see WO2010/115688), 141 (see WO2010/115688), 89 (see WO2010/115688), 54 (see WO2010/115688), 33 (see WO2010/115688), 17 (see WO2010/115688), 48 (see WO2010/115688) and 12 (see WO2010/115688) exhibited MIC₁₀₀ values of less than 5 μM against L4 larvae of Dirofilaria immitis.

### Definitions

The term “alkyl” (alone or in combination with another term(s)) means a straight- or branched-chain saturated hydrocarbyl substituent (i.e., a substituent containing only carbon and hydrogen) typically containing from 1 to about 20 carbon atoms, more typically from 1 to about 8 carbon atoms, and even more typically from 1 to about 6 carbon atoms. Examples of such substituents include methyl, ethyl, n-propyl, isopropyl, n-butyl, iso-butyl, sec-butyl, tert-butyl, pentyl, iso-amyl, hexyl, and octyl.

The term “alkenyl” (alone or in combination with another term(s)) means a straight- or branched-chain hydrocarbyl substituent containing one or more double bonds and typically from 2 to about 20 carbon atoms, more typically from about 2 to about 10 carbon atoms, even more typically from about 2 to about 8 carbon atoms, and still even more typically from about 2 to about 6 carbon atoms. Examples of such substituents include ethenyl (vinyl); 2-propenyl; 3-propenyl; 1,4-pentadienyl; 1,4-butadienyl; 1-butenyl; 2-butenyl; 3-butenyl; and decenyl.

The term “alkynyl” (alone or in combination with another term(s)) means a straight- or branched-chain hydrocarbyl substituent containing one or more triple bonds and typically from 2 to about 20 carbon atoms, more typically from about 2 to about 8 carbon atoms, and even more typically from about 2 to about 6 carbon atoms. Examples of such substituents include ethynyl, 2-propynyl, 3-propynyl, decynyl, 1-butynyl, 2-butynyl, and 3-butynyl.

The term “carbocyclyl” (alone or in combination with another term(s)) means a saturated cyclic (i.e., “cycloalkyl”), partially saturated cyclic (i.e., “cycloalkenyl”), or completely unsaturated (i.e., “aryl”) hydrocarbyl substituent typically containing from 3 to 14 carbon ring atoms (“ring atoms” are the atoms bound together to form the ring or rings of a cyclic moiety). A carbocyclyl may be a single ring, which typically contains from 3 to 6 ring atoms. Examples of such single-ring carbocyclyls include cyclopropanyl, cyclobutanyl, cyclopentyl, cyclopentenyl, cyclopentadienyl, cyclohexyl, cyclohexenyl, cyclohexadienyl, and phenyl. A carbocyclyl alternatively may be multiple (typically 2 or 3) rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluoreneyl, decalinyl, and norpinanyl.

The term “cycloalkyl” (alone or in combination with another term(s)) means a saturated cyclic hydrocarbyl substituent typically containing from 3 to 14 carbon ring atoms. A cycloalkyl may be a single carbon ring, which typically contains from 3 to 6 carbon ring atoms. Examples of single-ring cycloalkyls include cyclopropyl (or “cyclopropanyl”), cyclobutyl (or “cyclobutanyl”), cyclopentyl (or “cyclopentanyl”), and cyclohexyl (or “cyclohexanyl”). A cycloalkyl alternatively may be multiple (typically 2 or 3) carbon rings fused together, such as, decalinyl or norpinanyl.

The term “aryl” (alone or in combination with another term(s)) means an aromatic carbocyclyl typically containing from 6 to 14 carbon ring atoms. Examples of aryls include phenyl, naphthalenyl, and indenyl.

In some instances, the number of carbon atoms in a hydrocarbyl group (e.g., alkyl, alkenyl, alkynyl, or cycloalkyl) is indicated by the prefix “Cₓ-C_{y}-”, wherein x is the minimum and y is the maximum number of carbon atoms in the group. Thus, for example, “C₁-C₆-alkyl” refers to an alkyl substituent containing from 1 to 6 carbon atoms. Illustrating further, C₃-C₆-cycloalkyl means a saturated hydrocarbyl ring containing from 3 to 6 carbon ring atoms.

The term “hydrogen” (alone or in combination with another term(s)) means a hydrogen radical (or “hydrido”), and may be depicted as —H.

The term “hydroxy” (alone or in combination with another term(s)) means —OH.

The term “nitro” (alone or in combination with another term(s)) means —NO₂.

The term “cyano” (alone or in combination with another term(s)) means —CN, which also may be depicted:

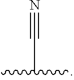 [SMILES: *C#N]

The term “oxo” (alone or in combination with another term(s)) means an oxo radical, and may be depicted as:

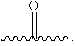 [SMILES: *=O]

The term “carboxy” (alone or in combination with another term(s)) means —C(O)—OH, which also may be depicted as:

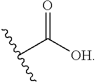 [SMILES: *C(=O)O]

The term “amino” (alone or in combination with another term(s)) means —NH₂.

The term “halogen” (alone or in combination with another term(s)) means a fluorine radical (“fluoro”, which may be depicted as —F), chlorine radical (“chloro”, which may be depicted as —Cl), bromine radical (“bromo”, which may be depicted as —Br), or iodine radical (“iodo”, which may be depicted as —I). Typically, fluoro or chloro is preferred, with fluoro often being particularly preferred.

If a substituent is described as being “substituted”, a non-hydrogen substituent is in the place of a hydrogen on a carbon, nitrogen, oxygen, or sulfur of the substituent. Thus, for example, a substituted alkyl substituent is an alkyl substituent wherein at least one non-hydrogen substituent is in the place of a hydrogen on the alkyl substituent. To illustrate, monofluoroalkyl is alkyl substituted with a fluoro, and difluoroalkyl is alkyl substituted with two fluoros. It should be recognized that if there are more than one substitutions on a substituent, each non-hydrogen substituent may be identical or different (unless otherwise stated).

If a substituent is described as being “optionally substituted”, the substituent may be either (1) not substituted or (2) substituted. If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 substituents, then any heteroaryl with less than 3 substitutable positions would be optionally substituted by up to only as many non-hydrogen substituents as the heteroaryl has substitutable positions. To illustrate, tetrazolyl (which has only one substitutable position when it is bonded to a single non-hydrogen moiety by a single bond) would be optionally substituted with up to one non-hydrogen substituent. To illustrate further, if an amino nitrogen is described as being optionally substituted with up to 2 non-hydrogen substituents, then a primary amino nitrogen will be optionally substituted with up to 2 non-hydrogen substituents, whereas a secondary amino nitrogen will be optionally substituted with up to only one non-hydrogen substituent.

The term “substitutable position” means a position where the substituent moiety provides a pharmacokinetic and pharmacodynamic stable compound for the intended use.

The prefix “halo” indicates that the substituent to which the prefix is attached is substituted with one or more independently selected halogens. For example, haloalkyl means an alkyl substituent having a halogen in the place of a hydrogen, or multiple halogens in the place of the same number of hydrogens. Examples of haloalkyls include chloromethyl, 1-bromoethyl, fluoromethyl, difluoromethyl, trifluoromethyl, and 1,1,1-trifluoroethyl. Illustrating further, “haloalkoxy” means an alkoxy substituent wherein a halogen is in the place of a hydrogen, or multiple halogens are in the place of the same number of hydrogens. Examples of haloalkoxy substituents include chloromethoxy, 1-bromoethoxy, fluoromethoxy, difluoromethoxy, trifluoromethoxy (also known as “perfluoromethyloxy”), and 1,1,1,-trifluoroethoxy. It should be recognized that if a substituent is substituted by more than one halogen, the halogens may be identical or different (unless otherwise stated).

The term “carbonyl” (alone or in combination with another term(s)) means —C(O)—, which also may be depicted as:

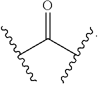 [SMILES: *C(*)=O]

This term also is intended to encompass a hydrated carbonyl substituent, i.e., —C(OH)₂—.

The term “aminocarbonyl” (alone or in combination with another term(s)) means —C(O)—NH₂, which also may be depicted as:

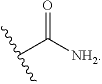 [SMILES: *C(N)=O]

The term “oxy” (alone or in combination with another term(s)) means an ether substituent, and may be depicted as —O—.

The term “alkoxy” (alone or in combination with another term(s)) means an alkylether substituent, i.e., —O-alkyl. Examples of such a substituent include methoxy (—O—CH₃), ethoxy, n-propoxy, iso-propoxy, n-butoxy, iso-butoxy, sec-butoxy, and tert-butoxy.

The term “alkylcarbonyl” (alone or in combination with another term(s)) means —C(O)-alkyl. For example, “ethylcarbonyl” may be depicted as:

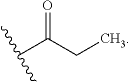 [SMILES: *C(=O)CC]

The term “alkoxycarbonyl” (alone or in combination with another term(s)) means —C(O)—O-alkyl. For example, “ethoxycarbonyl” may be depicted as:

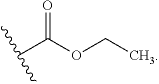 [SMILES: *C(=O)OCC]

The term “carbocyclylcarbonyl” (alone or in combination with another term(s)) means —C(O)-carbocyclyl. For example, “phenylcarbonyl” may be depicted as:

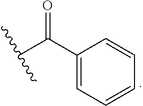 [SMILES: *C(=O)c1ccccc1]

Similarly, the term “heterocyclylcarbonyl” (alone or in combination with another term(s)) means —C(O)-heterocyclyl.

The term “sulfanyl” (alone or in combination with another term(s)) means a thiaether substituent, i.e., an ether substituent wherein a divalent sulfur atom is in the place of the ether oxygen atom. Such a substituent may be depicted as —S—. This, for example, “alkyl-sulfanyl-alkyl” means alkyl-S-alkyl.

The term “thiol” or “mercapto” (alone or in combination with another term(s)) means a sulfhydryl substituent, and may be depicted as —SH.

The term “thiocarbonyl” (alone or in combination with another term(s)) means a carbonyl wherein a sulfur is in the place of the oxygen. Such a substituent may be depicted as —C(S)—, and also may be depicted as:

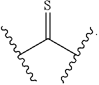 [SMILES: *C(*)=S]

The term “sulfonyl” (alone or in combination with another term(s)) means —S(O)₂—, which also may be depicted as:

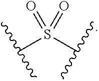 [SMILES: *S(*)(=O)=O]

Thus, for example, “alkyl-sulfonyl-alkyl” means alkyl-S(O)₂-alkyl.

The term “aminosulfonyl” (alone or in combination with another term(s)) means —S(O)₂—NH₂, which also may be depicted as:

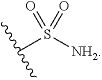 [SMILES: *S(N)(=O)=O]

The term “sulfinyl” (alone or in combination with another term(s)) means —S(O)—, which also may be depicted as:

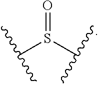 [SMILES: *S(*)=O]

Thus, for example, “alkyl-sulfinyl-alkyl” means alkyl-S(O)-alkyl.

The term “heterocyclyl” (alone or in combination with another term(s)) means a saturated (i.e., “heterocycloalkyl”), non-aromatic partially-saturated (i.e., “heterocycloalkenyl”), or heterocyclic aromatic (i.e., “heteroaryl”) ring structure typically containing a total of 3 to 14 ring atoms. At least one of the ring atoms is a heteroatom (typically oxygen, nitrogen, or sulfur), with the remaining ring atoms generally being independently selected from the group typically consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, thienyl (also known as “thiophenyl” and “thiofuranyl”), oxazolyl, isoxazolyl, thiazolyl, isothiazolyl, thiodiazolyl, oxadiazolyl (including 1,2,3-oxadiazolyl, 1,2,4-oxadiazolyl (also known as “azoximyl”), 1,2,5-oxadiazolyl (also known as “furazanyl”), and 1,3,4-oxadiazolyl), pyrrolyl, pyrazolyl, imidazolyl, triazolyl, tetrazolyl, oxathiazolyl, oxatriazolyl (including 1,2,3,4-oxatriazolyl and 1,2,3,5-oxatriazolyl), pyridinyl, diazinyl (including pyridazinyl (also known as “1,2-diazinyl”), pyrimidinyl (also known as “1,3-diazinyl”), and pyrazinyl (also known as “1,4-diazinyl”)), triazinyl (including s-triazinyl (also known as “1,3,5-triazinyl”), as-triazinyl (also known 1,2,4-triazinyl), and v-triazinyl (also known as “1,2,3-triazinyl”)), oxathiazinyl (including 1,2,5-oxathiazinyl and 1,2,6-oxathiazinyl), oxepinyl, thiepinyl, dihydrofuranyl, tetrahydrofuranyl, dihydrothienyl (also known as “dihydrothiophenyl”), tetrahydrothienyl (also known as “tetrahydrothiophenyl”), isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl (including 1,2,3-dioxazolyl, 1,2,4-dioxazolyl, 1,3,2-dioxazolyl, and 1,3,4-dioxazolyl), pyranyl (including 1,2-pyranyl and 1,4-pyranyl), dihydropyranyl, tetrahydropyranyl, piperidinyl, piperazinyl, oxazinyl (including 1,2,3-oxazinyl, 1,3,2-oxazinyl, 1,3,6-oxazinyl (also known as “pentoxazolyl”), 1,2,6-oxazinyl, and 1,4-oxazinyl), isoxazinyl (including o-isoxazinyl and p-isoxazinyl), oxadiazinyl (including 1,4,2-oxadiazinyl and 1,3,5,2-oxadiazinyl), morpholinyl, azepinyl, and diazepinyl.

A heterocyclyl alternatively may be 2 or 3 rings fused together, such as, for example, indolizinyl, pyranopyrrolyl, purinyl, imidazopyrazinyl, imidazolopyridazyl, pyridopyridinyl (including pyrido[3,4-b]-pyridinyl, pyrido[3,2-b]-pyridinyl, pyrido[4,3-b]-pyridinyl, and naphthyridinyl), pteridinyl, pyridazinotetrazinyl, pyrazinotetrazinyl, pyrimidinotetrazinyl, pyrindinyl, pyrazolopyrimidinyl, pyrazolopyrazinyl, pyrazolopyridazyl, or 4H-quinolizinyl. In some embodiments, the preferred multi-ring heterocyclyls are indolizinyl, pyranopyrrolyl, purinyl, pyridopyridinyl, pyrindinyl, and 4H-quinolizinyl.

Other examples of fused-ring heterocyclyls include benzo-fused heterocyclyls, such as, for example, benzofuranyl (also known as “coumaronyl”), isobenzofuranyl, benzoxazolyl, benzoisoxazolyl (also known as “indoxazinyl”), anthranilyl, benzothienyl (also known as “benzothiophenyl”, “thionaphthenyl”, and “benzothiofuranyl”), isobenzothienyl (also known as “isobenzothiophenyl”, “isothionaphthenyl”, and “isobenzothiofuranyl”), benzothiazolyl, benzoisothiazolyl, benzothiadiazolyl, benzoxadiazolyl, indolyl, isoindazolyl (also known as “benzpyrazolyl”), benzoimidazolyl, benzotriazolyl, benzazinyl (including quinolinyl (also known as “1-benzazinyl”) and isoquinolinyl (also known as “2-benzazinyl”)), phthalazinyl, quinoxalinyl, benzodiazinyl (including cinnolinyl (also known as “1,2-benzodiazinyl”) and quinazolinyl (also known as “1,3-benzodiazinyl”)), benzoimidazothiazolyl, carbazolyl, acridinyl, isoindolyl, indoleninyl (also known as “pseudoindolyl”), benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, benzoxazinyl (including 1,3,2-benzoxazinyl, 1,4,2-benzoxazinyl, 2,3,1-benzoxazinyl, and 3,1,4-benzoxazinyl), benzoisoxazinyl (including 1,2-benzisoxazinyl and 1,4-benzisoxazinyl), benzoxadiazinyl, and xanthenyl. In some embodiments, the preferred benzo-fused heterocyclyls are benzofuranyl, isobenzofuranyl, benzoxazolyl, benzoisoxazolyl, anthranilyl, benzothienyl, isobenzothienyl, benzothiazolyl, benzothiadiazolyl, benzoxadiazolyl, indolyl, isoindazolyl, benzoimidazolyl, benzotriazolyl, benzazinyl, phthalazinyl, quinoxalinyl, benzodiazinyl, carbazolyl, acridinyl, isoindolyl, indoleninyl, benzodioxolyl, chromanyl, isochromanyl, thiochromanyl, benzodioxanyl, tetrahydroisoquinolinyl, benzoxazinyl, benzoisoxazinyl, and xanthenyl.

The term “2-fused-ring” heterocyclyl (alone or in combination with another term(s)) means a saturated, non-aromatic partially-saturated, or heteroaryl containing two fused rings. Such heterocyclyls include, for example, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, and benzoisoxazinyl. In some embodiments, preferred 2-fused-ring heterocyclyls include benzofuranyl, isobenzofuranyl, benzoxazolyl, benzoisoxazolyl, anthranilyl, benzothienyl, isobenzothienyl, benzothiazolyl, benzothiadiazolyl, indolizinyl, pyranopyrrolyl, benzoxadiazolyl, indolyl, isoindazolyl, benzoimidazolyl, benzotriazolyl, purinyl, quinolinyl, isoquinolinyl, pyridopyridinyl, phthalazinyl, quinoxalinyl, benzodiazinyl, pteridinyl, pyrindinyl, isoindolyl, indoleninyl, benzodioxolyl, benzodioxanyl, tetrahydroisoquinolinyl, 4H-quinolizinyl, benzoxazinyl, and benzoisoxazinyl.

The term “heteroaryl” (alone or in combination with another term(s)) means an aromatic heterocyclyl typically containing from 5 to 14 ring atoms. A heteroaryl may be a single ring or multiple (typically 2 or 3) fused rings. Such moieties include, for example, 5-membered rings such as furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, and oxatriazolyl; 6-membered rings such as pyridinyl, pyrazinyl, pyrimidinyl, pyridazinyl, triazinyl, and oxathiazinyl; 7-membered rings such as oxepinyl and thiepinyl; 6/5-membered fused-ring systems such as benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, and imidazolopyridazyl; and 6/6-membered fused-ring systems such as quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, and acridinyl. In some embodiments, the preferred 5-membered rings include furanyl, thienyl, oxazolyl, isoxazolyl, thiazolyl, isothiazolyl, oxadiazolyl, pyrazolyl, and imidazolyl; the preferred 6-membered rings include pyridinyl, pyrazinyl, pyrimidinyl, pyridazinyl, and triazinyl; the preferred 6/5-membered fused-ring systems include benzoxazolyl, benzoisoxazolyl, anthranilyl, benzothienyl, isobenzothienyl, and purinyl; and the preferred 6/6-membered fused-ring systems include quinolinyl, isoquinolinyl, and benzodiazinyl.

A carbocyclyl or heterocyclyl can optionally be substituted with, for example, one or more substituents independently selected from the group consisting of halogen, hydroxy, carboxy, oxo, alkyl, alkoxy, alkoxyalkyl, alkylcarbonyl, aryl, arylalkyl, arylalkoxy, arylalkoxyalkyl, arylalkoxycarbonyl, cycloalkyl, cycloalkylalkyl, cycloalkylalkoxy, cycloalkylalkoxyalkyl, and cycloalkylalkoxycarbonyl. More typically, a carbocyclyl or heterocyclyl may optionally be substituted with, for example, one or more substituents independently selected from the group consisting of halogen, —OH, —C(O)—OH, oxo, C₁-C₆-alkyl, C₁-C₆-alkoxy, C₁-C₆-alkoxy-C₁-C₆-alkyl, C₁-C₆-alkylcarbonyl, aryl, aryl-C₁-C₆-alkyl, aryl-C₁-C₆-alkoxy, aryl-C₁-C₆-alkoxy-C₁-C₆-alkyl, aryl-C₁-C₆-alkoxycarbonyl, cycloalkyl, cycloalkyl-C₁-C₆-alkyl, cycloalkyl-C₁-C₆-alkoxy, cycloalkyl-C₁-C₆-alkoxy-C₁-C₆-alkyl, and cycloalkyl-C₁-C₆-alkoxycarbonyl. The alkyl, alkoxy, alkoxyalkyl, alkylcarbonyl, aryl, arylalkyl, arylalkoxy, arylalkoxyalkyl, or arylalkoxycarbonyl substituent(s) may further be substituted with, for example, one or more halogen. The aryl and cycloalkyl portions of such optional substituents are typically single-rings containing from 3 to 6 ring atoms, and more typically from 5 to 6 ring atoms.

An aryl or heteroaryl can optionally be substituted with, for example, one or more substituents independently selected from the group consisting of halogen, —OH, —CN, —NO₂, —SH, —C(O)—OH, amino, aminoalkyl, alkyl, alkylsulfanyl, carboxyalkylsulfanyl, alkylcarbonyloxy, alkoxy, alkoxyalkyl, alkoxycarbonylalkoxy, alkoxyalkylsulfanyl, alkoxycarbonylalkylsulfanyl, carboxyalkoxy, alkoxycarbonylalkoxy, carbocyclyl, carbocyclylalkyl, carbocyclyloxy, carbocyclylsulfanyl, carbocyclylalkylsulfanyl, carbocyclylamino, carbocyclylalkylamino, carbocyclylcarbonylamino, carbocyclylalkyl, carbocyclylcarbonyloxy, carbocyclyloxyalkoxycarbocyclyl, carbocyclylsulfanylalkylsulfanylcarbocyclyl, carbocyclylsulfanylalkoxycarbocyclyl, carbocyclyloxyalkylsulfanylcarbocyclyl, heterocyclyl, heterocyclylalkyl, heterocyclyloxy, heterocyclylsulfanyl, heterocyclylalkylsulfanyl, heterocyclylamino, heterocyclylalkylamino, heterocyclylcarbonylamino, heterocyclylcarbonyloxy, heterocyclyloxyalkoxyheterocyclyl, heterocyclylsulfanylalkylsulfanylheterocyclyl, heterocyclylsulfanylalkoxyheterocyclyl, and heterocyclyloxyalkylsulfanylheterocyclyl. More typically, an aryl or heteroaryl may, for example, optionally be substituted with one or more substituents independently selected from the group consisting of halogen, —OH, —CN, —NO₂, —SH, —C(O)—OH, amino, amino-C₁-C₆-alkyl, C₁-C₆-alkyl, C₁-C₆-alkylsulfanyl, carboxy-C₁-C₆-alkylsulfanyl, C₁-C₆-alkylcarbonyloxy, C₁-C₆-alkoxy, C₁-C₆-alkoxy-C₁-C₆-alkyl, C₁-C₆-alkoxycarbonyl-C₁-C₆-alkoxy, C₁-C₆-alkoxy-C₁-C₆-alkylsulfanyl, C₁-C₆-alkoxycarbonyl-C₁-C₆-alkylsulfanyl, carboxy-C₁-C₆-alkoxy, C₁-C₆-alkoxycarbonyl-C₁-C₆-alkoxy, aryl, aryl-C₁-C₆-alkyl, aryloxy, arylsulfanyl, aryl-C₁-C₆-alkylsulfanyl, arylamino, aryl-C₁-C₆-alkylamino, arylcarbonylamino, arylcarbonyloxy, aryloxy-C₁-C₆-alkoxyaryl, arylsulfanyl-C₁-C₆-alkylsulfanylaryl, arylsulfanyl-C₁-C₆-alkoxyaryl, aryloxy-C₁-C₆-alkylsulfanylaryl, cycloalkyl, cycloalkyl-C₁-C₆-alkyl, cycloalkyloxy, cycloalkylsulfanyl, cycloalkyl-C₁-C₆-alkylsulfanyl, cycloalkylamino, cycloalkyl-C₁-C₆-alkylamino, cycloalkylcarbonylamino, cycloalkylcarbonyloxy, heteroaryl, heteroaryl-C₁-C₆-alkyl, heteroaryloxy, heteroarylsulfanyl, heteroaryl-C₁-C₆-alkylsulfanyl, heteroarylamino, heteroaryl-C₁-C₆-alkylamino, heteroarylcarbonylamino, and heteroarylcarbonyloxy. Here, one or more hydrogens bound to a carbon in any such substituent may, for example, optionally be replaced with halogen. In addition, any cycloalkyl, aryl, and heteroaryl portions of such optional substituents are typically single-rings containing 3 to 6 ring atoms, and more typically 5 or 6 ring atoms.

A prefix attached to a multi-component substituent only applies to the first component. To illustrate, the term “alkylcycloalkyl” contains two components: alkyl and cycloalkyl. Thus, the C₁-C₆- prefix on C₁-C₆-alkylcycloalkyl means that the alkyl component of the alkylcycloalkyl contains from 1 to 6 carbon atoms; the C₁-C₆- prefix does not describe the cycloalkyl component.

If substituents are described as being “independently selected,” each substituent is selected independent of the other. Each substituent, therefore, may be identical to or different from the other selected substituent(s).

When words are used to describe a substituent, the rightmost-described component of the substituent is the component that has the free valence. To illustrate, benzene substituted with methoxyethyl has the following structure:

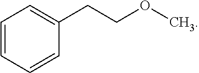

As can be seen, the ethyl is bound to the benzene, and the methoxy is the component of the substituent that is the component furthest from the benzene. As further illustration, benzene substituted with cyclohexanylsulfanylbutoxy has the following structure:

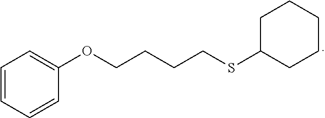 [SMILES: c1ccc(OCCCCSC2CCCCC2)cc1]

When a chemical formula is used to describe a mono-valent substituent, the dash on the left side of the formula indicates the portion of the substituent that has the free valence. To illustrate, benzene substituted with —C(O)—OH has the following structure:

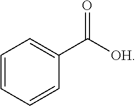

When a chemical formula is used to describe a di-valent (or “linking”) component between two other components of a depicted chemical structure (the right and left components), the leftmost dash of the linking component indicates the portion of the linking component that is bound to the left component in the depicted structure. The rightmost dash, on the other hand, indicates the portion of the linking component that is bound to the right component in the depicted structure. To illustrate, if the depicted chemical structure is X-L-Y and L is described as —C(O)—N(H)—, then the chemical would be:

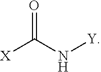

Dashes are not used to characterize a tri-valent component when standing alone. Thus, for example, a tri-valent nitrogen is identified as “N” and a tri-valent carbon bonded to hydrogen is identified as “CH” in this patent.

The words “comprise”, “comprises”, and “comprising” are to be interpreted inclusively rather than exclusively. This interpretation is intended to be the same as the interpretation that these words are given under United States patent law.

The term “pharmaceutically acceptable” is used adjectivally to mean that the modified noun is appropriate for use in a pharmaceutical product. When it is used, for example, to describe a salt or excipient, it characterizes the salt or excipient as being compatible with the other ingredients of the composition, and not deleterious to the intended recipient animal to the extent that the deleterious effect(s) outweighs the benefit(s) of the salt.

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

1. A method or treating an infection with Dirofilaria immitis, comprising administering to a subject animal a compound or salt thereof, wherein:
the compound corresponds in structure to Formula (I): 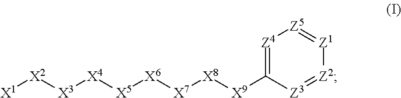
X¹ is selected from the group consisting of C₃-C₆-alkyl, C₃-C₆-alkenyl, C₃-C₆-alkynyl, cyclopentyl, cyclohexyl, phenyl, 5-member heterocycloalkyl, 5-member heterocycloalkenyl, 5-member heteroaryl, 6-member heterocycloalkyl, 6-member heterocycloalkenyl, and 6-member heteroaryl, wherein:
the C₃-C₆-alkyl, C₃-C₆-alkenyl, C₃-C₆-alkynyl, cyclopentyl, 5-member heterocycloalkyl, 5-member heterocycloalkenyl, and 5-member heteroaryl are optionally substituted by one or more substituents independently selected from the group consisting of halogen, cyano, alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl, wherein:
the alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl substituents are optionally substituted with one or more substituents independently selected from the group consisting of halogen, cyano, alkyl, alkoxy, haloalkyl, haloalkoxy, alkylsulfanyl, and haloalkylsulfanyl,
the cyclohexyl, phenyl, 6-member heterocycloalkyl, 6-member heterocycloalkenyl, and 6-member heteroaryl are optionally substituted by one or more substituents independently selected from the group consisting of halogen, cyano, alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl, wherein:
the alkyl, alkoxy, alkylsulfanyl, aryl, aryloxy, arylalkoxy, arylsulfanyl, arylalkylsulfanyl, heteroaryl, heteroaryloxy, heteroarylalkoxy, heteroarylsulfanyl, and heteroarylalkylsulfanyl substituents are optionally substituted with one or more substituents independently selected from the group consisting of halogen, cyano, alkyl, alkoxy, haloalkyl, haloalkoxy, alkylsulfanyl, and haloalkylsulfanyl;
X² is selected from the group consisting of a
bond, —O—, —C(O)—, —C(S)—, —NH—, —S—, —S(O)—, —S(O)₂—, —CH₂—, —CH₂CH₂—, —C(O)—CH₂—, —CH₂—C(O)—, —O—CH₂—, —CH₂—O—, —NH—CH₂—, —CH₂—NH—, —S—CH₂—, —CH₂—S—, —S(O)—CH₂—, —CH₂—S(O)—, —S(O)₂—CH₂—, and —CH₂—S(O)₂—, wherein:
the —NH— is optionally substituted with alkyl, and
the —CH₂—, —CH₂CH₂—, —C(O)—CH₂—, —CH₂—C(O)—, —O—CH₂—, —CH₂—O—, —NH—CH₂—, —CH₂—NH—, —S—CH₂—, —CH₂—S—, —S(O)—CH₂—, —CH₂—S(O)—, —S(O)₂—CH₂—, and —CH₂—S(O)₂— are optionally substituted with one or more independently selected alkyl;
X³ is a linker, wherein:
the linker is a hydrocarbon, wherein:
the linker comprises one or more nitrogen atoms, and
one or more of the carbons in the hydrocarbon are optionally substituted with one or more substituents independently selected from the group consisting of halogen, alkyl, alkoxy, and oxo, the linker comprises at least one chain of from 3 to 6 atoms that link X² to X⁴, wherein from 1 to 2 of the chain atoms are nitrogen, and
the linker comprises no chain of less than 3 atoms that links X² and X⁴;
X⁴ is selected from the group consisting of a bond, —CH₂—, —O—, —C(S)—, —C(O)—, —S(O)—, and —S(O)₂—, wherein:
the —CH₂— is optionally substituted with up to two substituents independently selected from the group consisting of alkyl, alkenyl, and carbocyclyl;
X⁵ is selected from the group consisting of a bond, —CH₂—, and carbocyclyl, wherein:
the —CH₂— is optionally substituted with up to two substituents independently selected from the group consisting of alkyl, alkenyl, and carbocyclyl;
X⁶ is selected from the group consisting of a bond, —CH₂—, and carbocyclyl, wherein:
the —CH₂— is optionally substituted with up to two substituents independently selected from the group consisting of alkyl, alkenyl, and carbocyclyl;
X⁷ is selected from the group consisting
of —CH₂—, —O—, —C(O)—, —C(S)—, —S—, —S(O)—, —S(O)₂—, —NH—, —C(O)—NH—, —C(S)—NH—, —NH—C(O)—, —NH—C(S)—, wherein:
the —CH₂— is optionally substituted with up to two substituents independently selected from the group consisting of alkyl, alkenyl, and carbocyclyl, and
any —NH— is optionally substituted at a substitutable position with a substituent selected from the group consisting of alkyl, alkenyl, alkynyl, alkoxyalkyl, carbocyclyl, and carbocyclylalkyl, wherein:
any such substituent is optionally substituted with one or more independently selected halogen;
X⁸ is selected from the group consisting of piperidinyl, piperazinyl, homopiperazinyl or pyrrolidinyl, wherein:
the piperidinyl, piperazinyl, homopiperazinyl or pyrrolidinyl is optionally substituted with one or more independently selected alkyl;
X⁴—X⁵—X⁶—X⁷ comprises no chain of less than 3 atoms that links X³ to X⁸;
X⁹ is selected from the group consisting of a bond, —O—, —C(O)—, —S—, —S(O)—, —S(O)₂—, and —NH—, wherein:
the —NH— is optionally substituted at a substitutable position with a substituent selected from the group consisting of alkyl, alkenyl, alkynyl, alkoxyalkyl, carbocyclyl, and carbocyclylalkyl, wherein:
any such substituent is optionally substituted with one or more independently selected halogen;
Z¹ is selected from the group consisting of N and CH, wherein:
the CH is optionally substituted with a substituent selected from the group consisting of halogen, nitro, cyano, aminosulfonyl, alkyl, alkoxy, alkoxycarbonyl, alkylsulfanyl, alkylsulfinyl, alkyl sulfonyl, aryl, arylsulfanyl, arylsulfinyl, aryl sulfonyl, heteroaryl, heteroarylsulfanyl, heteroarylsulfinyl, and heteroarylsulfonyl, wherein:
the alkyl, alkoxy, alkoxycarbonyl, alkylsulfanyl, alkylsulfinyl, alkylsulfonyl, aryl, arylsulfanyl, arylsulfinyl, arylsulfonyl, heteroaryl, heteroarylsulfanyl, heteroarylsulfinyl, and heteroarylsulfonyl are optionally substituted with one or more substituents independently selected from the group consisting of halogen and alkyl, and
the aminosulfonyl is optionally substituted with up to two independently selected alkyl;
Z² is selected from the group consisting of N and CH, wherein:
the CH is optionally substituted with a substituent selected from the group consisting of cyano, halogen, nitro, alkyl, alkoxy, haloalkyl, alkylsulfanyl, and haloalkylsulfanyl;
Z³, Z⁴, and Z⁵ are each independently selected from the group consisting of N and CH, wherein:
the CH is optionally substituted with a substituent selected from the group consisting of halogen, cyano, nitro, alkyl, alkoxy, alkylsulfanyl, haloalkyl, haloalkoxy, and haloalkylsulfanyl; and
only one of Z¹, Z², Z³, Z⁴, and Z⁵ may be N.

2. The method according to claim 1, wherein X³ is selected from the group of linkers consisting of:

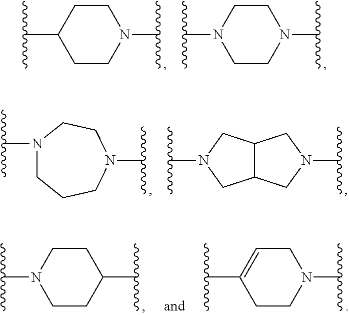

3. The method according to claim 1, wherein X³ is selected from the group of linkers consisting of:

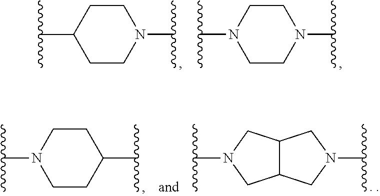

4. The method according to claim 1, wherein:
X¹ is selected from the group consisting of phenyl, 5-member heteroaryl, 6-member heteroaryl and C₃-C₆-alkyl wherein:
the 5-member heteroaryl is optionally substituted by one or more alkyl wherein:
the alkyl is optionally substituted with one or more independently selected halogen,
the phenyl and 6-member heteroaryl are optionally substituted at the meta and para positions by one or more substituents selected from the group consisting of alkyl, halogen, alkoxy, arylalkoxy, aryl, cyano and aryloxy wherein:
the alkyl and alkoxy are optionally substituted with one or more independently selected halogen;
the arylalkoxy is optionally substituted with one or more haloalkyl; and
the phenyl is optionally substituted at the ortho positions with one or two independently selected halogen;
X² is selected from the group consisting of a bond, —CH₂—O—, —C(O)—, —N(H)— and —C(S)—;
X⁴ is selected from the group consisting of a bond, —CH₂—, —O—, and —C(O)—, wherein:
the —CH₂— is optionally substituted with up to two independently selected alkyl;
X⁵ is selected from the group consisting of a bond and —CH₂—;
X⁶ is selected from the group consisting of a bond, —CH₂— and cycloalkyl wherein:
the —CH₂— is optionally substituted with up to two independently selected alkyl;
X⁷ is selected from the group consisting of —C(O)—, —C(S)—, —NH—C(O)—, —C(O)—NH—, —C(S)—NH—, —S(O)₂— and —C(O)—NH— wherein:
the —NH—C(O)— and —NH—C(S)— are optionally substituted with alkyl;
X⁸ is piperidinyl or pyrrolidinyl;
Z¹ is selected from the group consisting of N and CH, wherein:
the CH is optionally substituted with a substituent selected from the group consisting of nitro, halogen, cyano, alkyl, alkoxy, alkylsulfanyl, alkylsulfinyl, alkylsulfonyl, arylsulfonyl, heteroaryl, aminosulfonyl and alkoxycarbonyl wherein:
the alkyl, alkoxy, alkylsulfanyl, arylsulfonyl, heteroaryl and aminosulfonyl are optionally substituted with one or more substituents independently selected from the group consisting of halogen and alkyl;
Z² is selected from the group consisting of N and CH, wherein:
the CH is optionally substituted with a substituent selected from the group consisting of alkyl, halogen, cyano, alkoxy, haloalkyl, alkylsulfanyl and haloalkylsulfanyl;
Z³ and Z⁴ are independently selected from the group consisting of N and CH; and
Z⁵ is CH.

5. The method according to claim 1, wherein:
X¹ is selected from the group consisting of phenyl, 5-member heteroaryl, and 6-member heteroaryl, and C₃-C₆-alkyl wherein:
the 5-member heteroaryl is optionally substituted by one or more alkyl wherein:
the alkyl is optionally substituted with one or more independently selected halogen,
the phenyl and 6-member heteroaryl are optionally substituted at the meta and para positions by one or more substituents selected from the group consisting of alkyl, halogen, aryloxy, alkoxy, arylalkoxy and cyano wherein:
the alkyl is optionally substituted with one or more independently selected halogen;
the arylalkoxy is optionally substituted with one or more haloalkyl;
the phenyl is optionally substituted at the ortho position with one or more halogen;
X² is selected from the group consisting of a bond, —C(O)—, and —CH₂—O—;
X⁴ is selected from the group consisting of a bond, —CH₂—, —O—, and —C(O)—, wherein:
the —CH₂— is optionally substituted with up to two substituents independently selected alkyl;
X⁵ is selected from the group consisting of a bond and —CH₂—;
X⁶ is selected from the group consisting of a bond and —CH₂—, wherein:
the —CH₂— is optionally substituted with up to two substituents independently selected alkyl;
X⁷ is selected from the group consisting of —C(O)—, —C(S)—, —NH—C(O)—, —C(O)—NH—, S(O)₂, and —C(S)—NH— wherein:
the —NH—C(O)— is optionally substituted with alkyl;
X⁸ is selected from the group consisting of 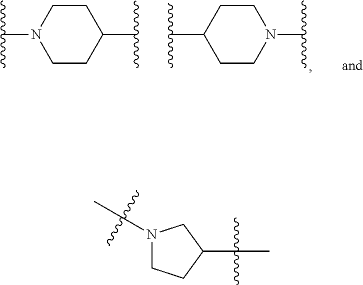
X⁹ is selected from the group consisting of a bond, —NH—, and —O;
Z¹ is selected from the group consisting of N and CH, wherein:
the CH is optionally substituted with a substituent selected from the group consisting of nitro, halogen, cyano, alkyl, alkoxy, alkylsulfinyl, alkylsulfanyl, alkyl sulfonyl, aryl sulfonyl, aminosulfonyl, and 5-membered heteroaryl, wherein:
the alkyl, alkoxy, alkylsulfanyl, arylsulfonyl, aminosulfonyl, and 5-membered heteroaryl are optionally substituted with one or more substituents independently selected from the group consisting of halogen and alkyl;
Z² is selected from the group consisting of N and CH, wherein:
the CH is optionally substituted with a substituent selected from the group consisting of alkyl, halogen, cyano, alkoxy, haloalkyl, alkylsulfanyl and haloalkylsulfanyl;
Z³ and Z⁴ are independently selected from the group consisting of N and CH;
Z⁵ is CH;

6. The method according to claim 1, wherein:
X¹ is selected from the group consisting of phenyl, 5-member heteroaryl, and 6-member heteroaryl, wherein:
the 5-member heteroaryl is optionally substituted by one or more alkyl wherein:
the alkyl is optionally substituted with one or more independently selected halogen,
the phenyl and 6-member heteroaryl are optionally substituted at the meta and para positions by one or more substituents selected from the group consisting of alkyl, halogen, aryloxy, alkoxy, and arylalkoxy wherein:
the alkyl is optionally substituted with one or more independently selected halogen;
the arylalkoxy is optionally substituted with one or more haloalkyl;
X² is selected from the group consisting of a bond and —CH₂—O—;
X³ is a linker selected from the group consisting of: 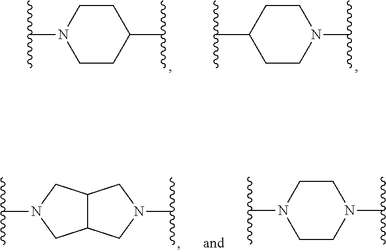 [SMILES: *C1CCN(*)CC1 ; *C1CCN(*)CC1 ; *N1CC2CN(*)CC2C1 ; *N1CCN(*)CC1]
X⁴ is selected from the group consisting of a bond, —CH₂—, —O—, and —C(O)—;
X⁵ is selected from the group consisting of a bond and —CH₂—;
X⁶ is selected from the group consisting of a bond and —CH₂—, wherein:
the —CH₂— is optionally substituted with up to two substituents independently selected alkyl;
X⁷ is selected from the group consisting of —C(O)—, —C(S)—, —NH—C(O)—, —C(O)—NH—, and —C(S)—NH—, wherein:
the —NH—C(O)— is optionally substituted with alkyl;
X⁸ is selected from the group consisting of 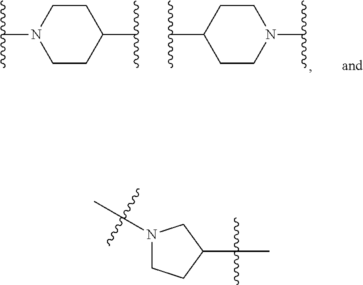
X⁹ is selected from the group consisting of a bond, —NH—, and —O;
Z¹ is selected from the group consisting of N and CH, wherein:
the CH is optionally substituted with a substituent selected from the group consisting of nitro, halogen, cyano, alkyl, alkoxy, alkylsulfanyl, alkylsulfonyl, arylsulfonyl and 5-membered-heteroaryl, wherein:
the alkyl, alkoxy, alkylsulfanyl, arylsulfonyl and 5-membered-heteroaryl are optionally substituted with one or more substituents independently selected from the group consisting of halogen and alkyl;
Z² is selected from the group consisting of N and CH, wherein:
the CH is optionally substituted with a substituent selected from the group consisting of alkyl, halogen, cyano, alkoxy, haloalkyl, alkylsulfanyl and haloalkylsulfanyl;
Z³ and Z⁴ are independently selected from the group consisting of N and CH; and
Z⁵ is CH.

7. The method according to claim 1, wherein: 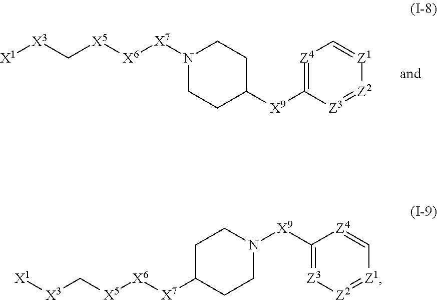
X¹ is selected from the group consisting of phenyl, 5-member heteroaryl, and 6-member heteroaryl, wherein:
the 5-member heteroaryl is optionally substituted with one or more independently selected alkyl; wherein:
the alkyl is optionally substituted with one or more independently selected halogen,
the phenyl and 6-member heteroaryl are optionally substituted at the meta and para positions by one or more substituents selected from the group consisting of alkyl and arylalkoxy wherein:
the alkyl is optionally substituted with one or more independently selected halogen;
the arylalkoxy is optionally substituted with one or more haloalkyl;
X³ is a linker selected from the group consisting of: 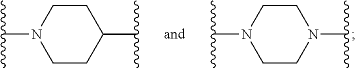 [SMILES: *C1CCN(*)CC1 ; *N1CCN(*)CC1]
X⁵ is selected from the group consisting of a bond and —CH₂—;
X⁶ is —CH₂—, wherein:
the —CH₂— is optionally substituted with up to two independently selected alkyl;
X⁷ is selected from the group consisting of —C(O)—, —C(S), —C(O)—NH—, and —C(S)—NH—;
X⁹ is selected from the group consisting of a bond, —NH—, and —O;
Z¹ is CH, wherein:
the CH is optionally substituted with a substituent selected from the group consisting of nitro, cyano, alkyl, alkylsulfanyl and alkylsulfonyl, wherein:
the alkyl and alkylsulfanyl are optionally substituted with one or more halogen;
Z² is CH, wherein:
the CH is optionally substituted with a substituent selected from the group consisting of alkyl, cyano, alkoxy and haloalkyl; and
Z³ and Z⁴ are independently selected from the group consisting of N and CH.

8. The method according to claim 1, wherein the compound corresponds in structure to: 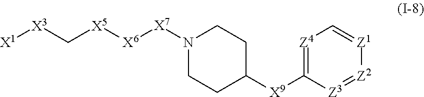
X⁹ is selected from the group consisting of —NH— and −O—.

9. The method according to claim 1, wherein the compound corresponds in structure to:

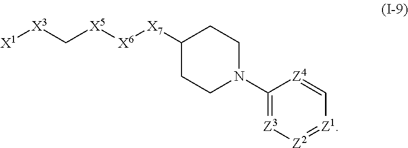

10. The method of claim 1, wherein: 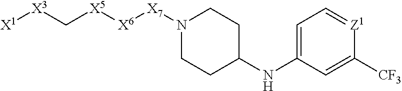
X¹ is selected from the group consisting of phenyl, 5-member heteroaryl, and 6-member heteroaryl, wherein:
the 5-member heteroaryl is optionally substituted with one or more independently selected haloalkyl;
the phenyl and 6-member heteroaryl are optionally substituted at the meta and para positions by one or more independently selected haloalkyl;
X³ is a linker selected from the group consisting of: 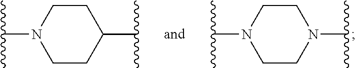 [SMILES: *C1CCN(*)CC1 ; *N1CCN(*)CC1]
X⁵ is selected from the group consisting of a bond and —CH₂—;
X⁶ is —CH₂—, wherein:
the —CH₂— is optionally substituted with up to two independently selected alkyl;
X⁷ is selected from the group consisting of —C(O)— and —C(S); and
Z¹ is CH optionally substituted with a substituent selected from the group consisting of nitro and cyano.

11. The method of claim 1, wherein the compound is selected from the group consisting of:

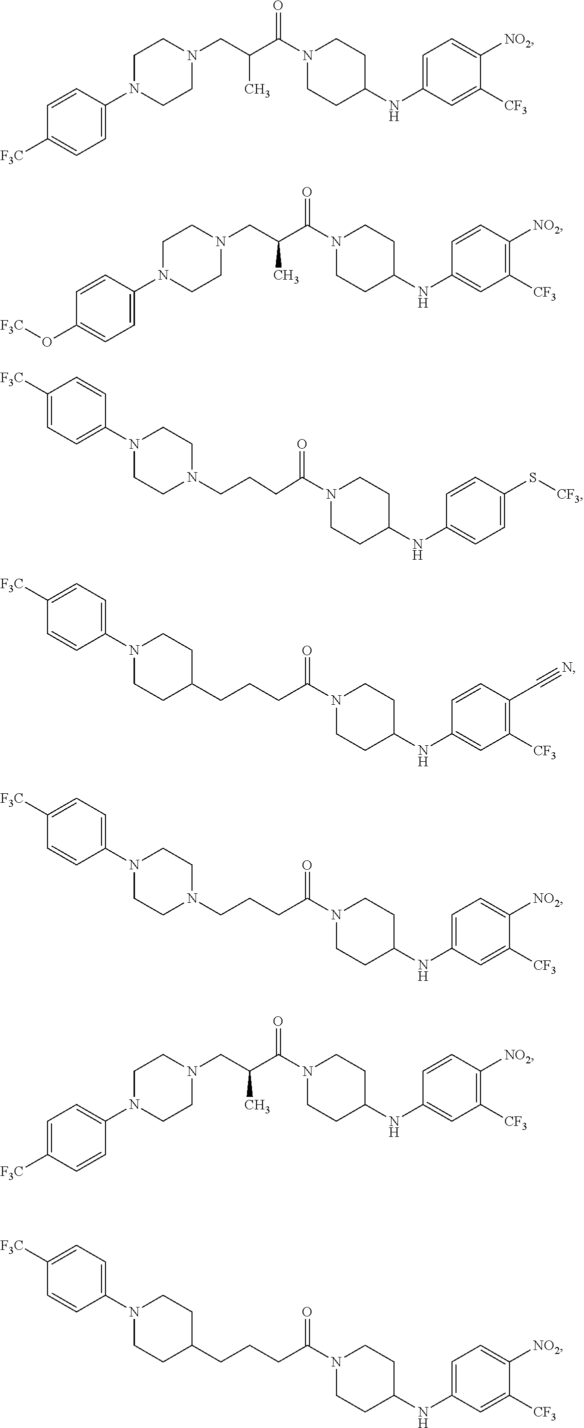

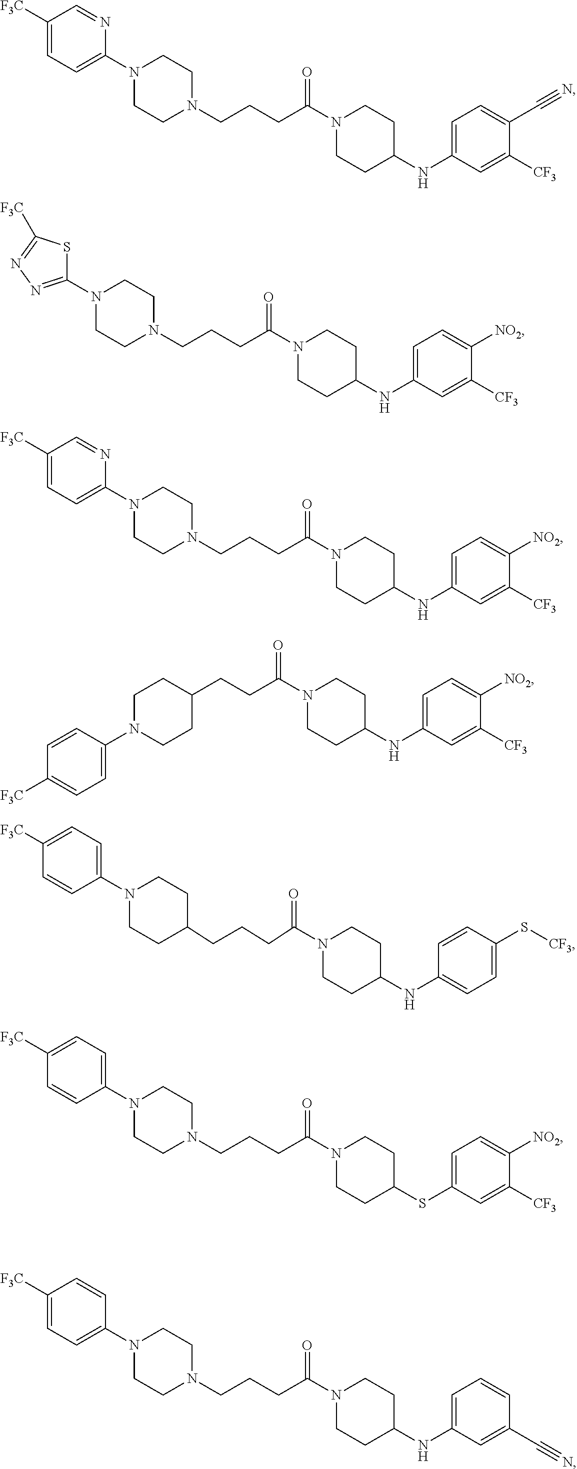

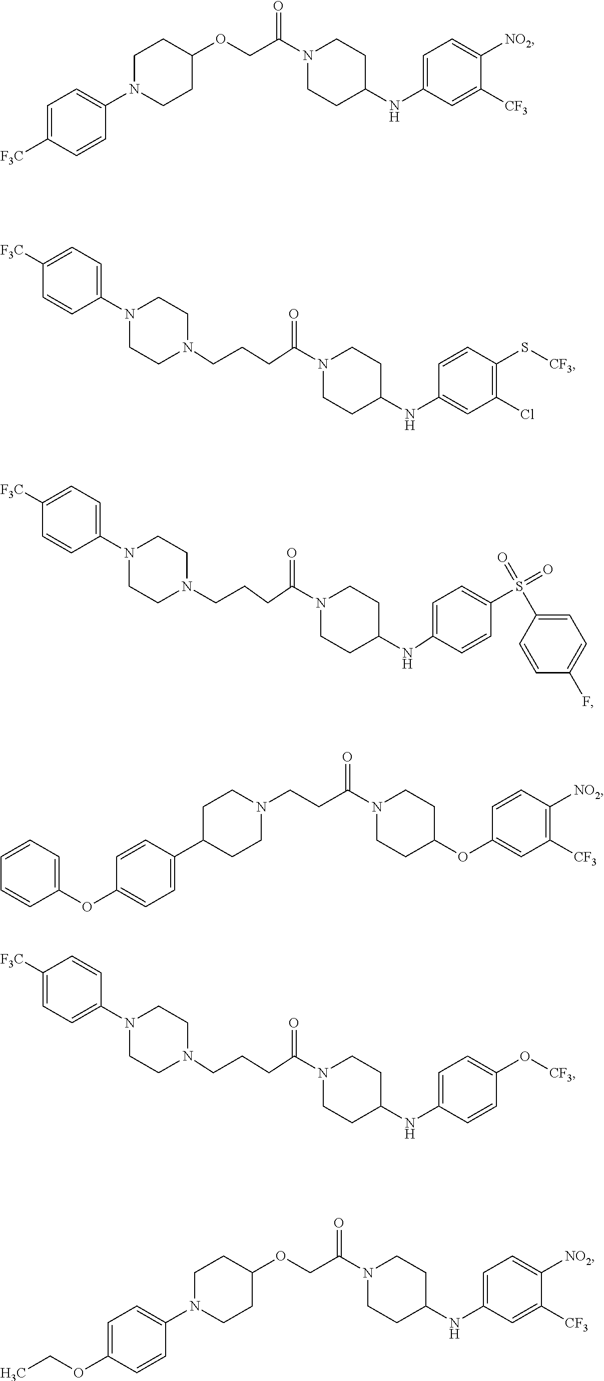 [SMILES: O=C(COC1CCN(c2ccc(C(F)(F)F)cc2)CC1)N1CCC(Nc2ccc([N+](=O)[O-])c(C(F)(F)F)c2)CC1 ; O=C(CCCN1CCN(c2ccc(C(F)(F)F)cc2)CC1)N1CCC(Nc2ccc(SC(F)(F)F)c(Cl)c2)CC1 ; O=C(CCCN1CCN(c2ccc(C(F)(F)F)cc2)CC1)N1CCC(Nc2ccc(S(=O)(=O)c3ccc(F)cc3)cc2)CC1 ; O=C(CCN1CCC(c2ccc(Oc3ccccc3)cc2)CC1)N1CCC(Oc2ccc([N+](=O)[O-])c(C(F)(F)F)c2)CC1 ; O=C(CCCN1CCN(c2ccc(C(F)(F)F)cc2)CC1)N1CCC(Nc2ccc(OC(F)(F)F)cc2)CC1 ; CCOc1ccc(N2CCC(OCC(=O)N3CCC(Nc4ccc([N+](=O)[O-])c(C(F)(F)F)c4)CC3)CC2)cc1]

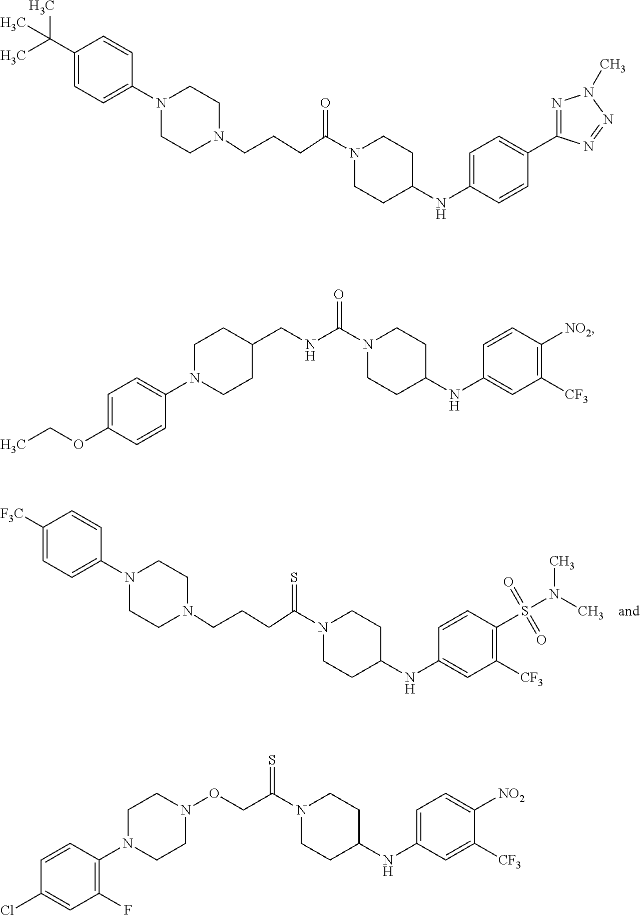 [SMILES: Cn1nnc(-c2ccc(NC3CCN(C(=O)CCCN4CCN(c5ccc(C(C)(C)C)cc5)CC4)CC3)cc2)n1 ; CCOc1ccc(N2CCC(CNC(=O)N3CCC(Nc4ccc([N+](=O)[O-])c(C(F)(F)F)c4)CC3)CC2)cc1 ; CN(C)S(=O)(=O)c1ccc(NC2CCN(C(=S)CCCN3CCN(c4ccc(C(F)(F)F)cc4)CC3)CC2)cc1C(F)(F)F ; "and" ; O=[N+]([O-])c1ccc(NC2CCN(C(=S)CON3CCN(c4ccc(Cl)cc4F)CC3)CC2)cc1C(F)(F)F]

12. The method of claim 1, wherein:
the compound corresponds in structure to Formula (I): 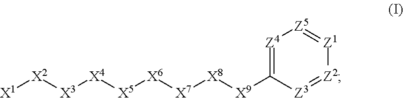
X¹ is selected from the group consisting of phenyl, 5-member heteroaryl and 6-member heteroaryl, wherein:
the 5-member heteroaryl is optionally substituted by alkyl, wherein:
the alkyl is optionally substituted with halogen,
the phenyl and 6-member heteroaryl are optionally substituted by one or more substituents independently selected from the group consisting of halogen, alkyl, alkoxy and aryloxy, wherein:
the alkyl and alkoxy substituents are optionally substituted with halogen;
X² is a bond;
X³ is selected from the group of linkers consisting of: 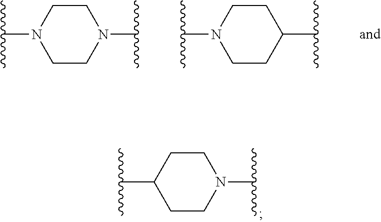
X⁴ is selected from the group consisting of a bond, —CH₂— and —O—;
X⁵ is selected from the group consisting of a bond and —CH₂—;
X⁶ is selected from the group consisting of a bond and —CH₂— wherein:
the —CH₂— is optionally substituted with an alkyl;
X⁷ is selected from the group consisting of —C(O)—, —C(S)— and —NH—C(O)—;
X⁸ is piperidinyl;
X⁴—X⁵—X⁶—X⁷ comprises no chain of less than 3 atoms that links X³ to X⁸;
X⁹ is selected from the group consisting of —O—, —S— and —NH—;
Z¹ is CH, wherein:
the CH is optionally substituted with a substituent selected from the group consisting of nitro, cyano, aminosulfonyl, alkoxy, alkylsulfanyl, arylsulfonyl and heteroaryl, wherein:
the alkoxy, alkylsulfanyl and arylsulfonyl are optionally substituted with one or more halogen and
the aminosulfonyl is optionally substituted with up to two independently selected alkyl;
Z² is CH, wherein:
the CH is optionally substituted with a substituent selected from the group consisting of cyano, halogen and haloalkyl;
Z³, Z⁴, and Z⁵ are CH.

13. The method of claim 1, wherein the compound or salt is active against larval worms and/or microfilariae of Dirofilaria immitis.

14. The method of claim 1, wherein the compound or salt is administered to a dog.

15. The method according to claim 1, wherein the compound or salt is administered in combination with at least one other component selected from the group consisting of an excipient and an active ingredient.

16. A kit, wherein the kit comprises:
at least one compound or salt of claim 1 and instructions to use in treating an infection with Dirofilaria immitis, and
at least one other component selected from the group consisting of an excipient, an active ingredient, instructions for combining the compound or salt with an excipient or active ingredient, an apparatus for combining the compound or salt with an excipient or active ingredient, instructions for administering the compound or salt to an animal, an apparatus for administering the compound or salt to an animal, and a diagnostic tool.

17. The kit according to claim 16, wherein the excipient comprises a solid dispersion of a polymer or graft copolymer.
